(19) United States
(12) Patent Application Publication
(10) Pub. No.: US 2011/0130406 A1
(43) Pub. Date: Jun. 2, 2011

(54) PYRAZOLO-PYRIDINES AS TYROSINE KINASE INHIBITORS

(75) Inventors: Jason DeMeese, Firestone, CO (US); John Gaudino, Longmont, CO (US); Alicia Tarin Neitzel, Longmont, CO (US); Paul Lunghofer, Longmont, CO (US); Jeongbeob Seo, Broomfield, CO (US); Hongqi Tian, Longmont, CO (US); Wendy B. Young, San Mateo, CA (US); Daniel P. Sutherlin, Burlingame, CA (US)

(73) Assignees: Array BioPharma Inc., Boulder, CO (US); Genentech, Inc., South San Francisco, CA (US)

(21) Appl. No.: 12/676,790

(22) PCT Filed: Sep. 5, 2008
(86) PCT No.: PCT/US08/75477

§ 371 Date: Feb. 17, 2011

Related U.S. Application Data
(60) Provisional application No. 60/970,472, filed on Sep. 6, 2007.

(51) Int. Cl. A61K 31/501; C07D 471/04; A61K 31/5377; A61K 31/497; A61K 31/4545; A61P 35/00

(52) U.S. Cl. 514/252.04; 544/238; 544/114; 544/405; 546/119; 514/234.2; 514/255.05; 514/303

(57) ABSTRACT

Compounds of Formulas Ia and Ib, and stereoisomers, geometric isomers, tautomers, solvates, metabolites and pharmaceutically acceptable salts thereof, are useful for inhibiting receptor tyrosine kinases and for treating disorders mediated thereby. Methods of using compounds of Formula Ia and Ib, and stereoisomers, geometric isomers, tautomers, solvates and pharmaceutically acceptable salts thereof, for in vitro, in situ, and in vivo diagnosis, prevention or treatment of such disorders in mammalian cells, or associated pathological conditions are disclosed.

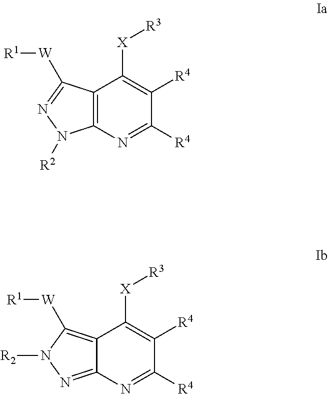

### PRIORITY OF THE INVENTION

This application claims priority to U.S. Provisional Application No. 60/970,472 that was filed 6 Sep. 2007, which is incorporated herein by reference in its entirety.

### FIELD OF THE INVENTION

The invention relates to heterobicyclic pyrazole compounds having protein tyrosine kinase activity. The heterobicyclic pyrazole compounds may be useful in the treatment of hyperproliferative disorders, such as cancer, in mammals. The invention also relates to pharmaceutical compositions and formulations, methods of synthesis, and methods of use such as treating hyperproliferative disorders.

### BACKGROUND OF THE INVENTION

Met tyrosine kinase is a high-affinity transmembrane receptor for the hepatocyte growth factor (HGF, Bottaro et al. (1991) Science 251:802-804). Met was cloned, named (Cooper et al. (1984) 311:29-33) and identified as an oncogene (Park et al. (1986) Cell 45:895-904). When deregulated by overexpression or mutations, Met receptor tyrosine kinase leads to tumor growth and invasion (Cristiani et al. (2005) Biochem. 44:14110-14119). Stimulation of Met by the ligand HGF, also known as Scatter Factor, initiates numerous physiological processes, including cell proliferation, scattering, morphogenic differentiation, angiogenesis, wound healing, tissue regeneration, and embryological development (Parr et al. (2004) Clin. Cancer Res. 10(1, Pt. 1) 202-211; Comoglio et al. (2002) J. Clin. Invest. 109:857-862; Maulik et al. (2002) Cytokine Growth Factor Reviews 13:41-59; Hecht et al. (2004) Cancer Res. 64(17):6109-6118). Receptor c-Met is rapidly internalized via clathrin-coated vesicles and traffics through an early endosomal compartment after hepatocyte growth factor stimulation. c-Met accumulates progressively in perinuclear compartments, which in part include the Golgi (Kermorgant et al. (2003) J. of Biol. Chem. 278(31):28921-28929).

The phenomena of: deregulation or dysregulation of Met and/or HGF; Met overexpression; and Met mutations are implicated in uncontrolled cell proliferation and survival, and play a key role in early-stage tumorigenesis, invasive growth of cancer cells, and metastasis (Danilkovitch-Miagkova et al. (2002) J. Clin. Invest. 109(7):863-867; Di Renzo et al. (1994) Int. J. Cancer 58:658-662; Matsumoto et al. (1994) J. Biol. Chem. 269:31807-31813; Tusolino et al. (1998) J. Cell Biol. 142:1145-1156; Jeffers et al. (1996) Mol. Cell. Biol. 16:1115-1125; Wong et al. (2004) Exper. Cell Res. 299(1):248-256; Konda et al. (2004) J. Urology 171(6), Pt. 1:2166-2170; Heideman et al. (2004) J. Gene Med. 6(3):317-327; Ma et al. (2003) Cancer Res. 63(19):6272-6281; Maulik et al. (2002) Clin. Cancer Res. 8:620-627), making Met an important target for anticancer drug development (Cohen, P. (2002) Nat. Rev. Drug Discovery 1:309-315). Overexpression of Met and HGF is associated with poor prognosis.

Recent data demonstrating the suppression of cancer cell proliferation, survival, and invasion upon inhibition of Met binding to HGF and Met receptor dimerization (Furge et al. (2001) Proc. Natl. Acad. Sci. USA 98:10722-10727; Michieli et al. (2004) Cancer Cell 6:61-73) confirm the relevance of Met in neoplasia and provide further proof of concept for the development of small-molecule compounds for antineoplastic therapy, e.g. against multiple myeloma (Hov et al. (2004) Clin. Cancer Res. 10(19):6686-6694). Inhibition of Met results in slowing tumor growth in tumor xenograft mouse models. Antibodies specific for c-Met have been expressed to block binding of HGF to c-Met (US 2005/0037431; US 2004/0166544).

Protein kinases (PK) are enzymes that catalyze the phosphorylation of hydroxy groups on tyrosine, serine and threonine residues of proteins by transfer of the terminal (gamma) phosphate from ATP. Through signal transduction pathways, these enzymes modulate cell growth, differentiation and proliferation, i.e., virtually all aspects of cell life in one way or another depend on PK activity. Furthermore, abnormal PK activity has been related to a host of disorders, ranging from relatively non-life threatening diseases such as psoriasis to extremely virulent diseases such as glioblastoma (brain cancer). Protein kinases include two classes; protein tyrosine kinases (PTK) and serine-threonine kinases (STK).

One of the prime aspects of PTK activity is their involvement with growth factor receptors which are cell-surface proteins. When bound by a growth factor ligand, growth factor receptors are converted to an active form which interacts with proteins on the inner surface of a cell membrane. This leads to phosphorylation on tyrosine residues of the receptor and other proteins and to the formation inside the cell of complexes with a variety of cytoplasmic signaling molecules that, in turn, effect numerous cellular responses such as cell division (proliferation), cell differentiation, cell growth, expression of metabolic effects to the extracellular microenvironment, etc. For a more complete discussion, see Schlessinger and Ullrich, (1992) Neuron 9:303-391.

Growth factor receptors with PTK activity are known as receptor tyrosine kinases (RTK, Plowman et al. (1994) DN&P, 7(6):334-339), which comprise a large family of transmembrane receptors with diverse biological activity. At present, at least nineteen (19) distinct subfamilies of RTK have been identified. An example of these is the subfamily designated the “HER” RTK, which include EGFR (epithelial growth factor receptor), HER2, HER3 and HER4. These RTK consist of an extracellular glycosylated ligand binding domain, a transmembrane domain and an intracellular cytoplasmic catalytic domain that can phosphorylate tyrosine residues on proteins. Another RTK subfamily consists of insulin receptor (IR), insulin-like growth factor I receptor (IGF-1R) and insulin receptor related receptor (IRR). IR and IGF-1R interact with insulin, IGF-I and IGF-II to form a heterotetramer of two entirely extracellular glycosylated alpha subunits and two beta subunits which cross the cell membrane and which contain the tyrosine kinase domain. A third RTK subfamily is referred to as the platelet derived growth factor receptor (PDGFR) group, which includes PDGFR-alpha, PDGFR-beta, CSFIR, c-kit and c-fms. These receptors consist of glycosylated extracellular domains composed of variable numbers of immunoglobin-like loops and an intracellular domain wherein the tyrosine kinase domain is interrupted by unrelated amino acid sequences. Another group which, because of its similarity to the PDGFR subfamily, is sometimes subsumed into the later group is the fetus liver kinase (flk) receptor subfamily. This group is believed to be made up of kinase insert domain-receptor fetal liver kinase-1 (KDR/FLK-1), flk-1R, flk-4 and fms-like tyrosine kinase 1 (flt-1). Another member of the tyrosine kinase growth factor receptor family is the fibroblast growth factor (“FGF”) receptor subgroup. This group consists of four receptors, FGFR1-4, and seven ligands, FGF1-7. While not yet well defined, it appears that these receptors consist of a glycosylated extracellular domain containing a variable number of immunoglobin-like loops and an intracellular domain in which the tyrosine kinase sequence is interrupted by regions of unrelated amino acid sequences. Still another member of the tyrosine kinase growth factor receptor family is the vascular endothelial growth factor (VEGF) receptor subgroup. VEGF is a dimeric glycoprotein similar to PDGF but has different biological functions and target cell specificity in vivo. In particular, VEGF is presently thought to play an essential role is vasculogenesis and angiogenesis.

Met is still another member of the tyrosine kinase growth factor receptor family, and often referred to as c-Met or human hepatocyte growth factor receptor tyrosine kinase (hHGFR). The expression of c-Met is thought to play a role in primary tumor growth and metastasis (Kim et al. Clin. Cancer Res. (2003) 9(14):5161-5170).

Modulation of the HGF/c-Met signaling pathway may be effected by regulating binding of HGF beta chain to cMet. In particular embodiments, the zymogen-like form of HGF beta mutant was shown to bind Met with 14-fold lower affinity than the wild-type serine protease-like form, suggesting optimal interactions result from conformational changes upon cleavage of the single-chain form (US 2005/0037431). Extensive mutagenesis of the HGF beta region corresponding to the active site and activation domain of serine proteases showed that 17 of the 38 purified two-chain HGF mutants resulted in impaired cell migration or Met phosphorylation but no loss in Met binding. However, reduced biological activities were well correlated with reduced Met binding of corresponding mutants of HGF beta itself in assays eliminating dominant alpha-chain binding contributions.

Protein-tyrosine kinases (PTK) are critical components of signaling pathways that control cellular proliferation and differentiation. PTK are subdivided into two large families, receptor tyrosine kinases (RTK) and non-receptor tyrosine kinases (NRTK). RTK span the plasma membrane and contain an extra-cellular domain, which binds ligand, and an intracellular portion, which possesses catalytic activity and regulatory sequences. Most RTK, like the hepatocyte growth factor receptor c-met, possess a single polypeptide chain and are monomeric in the absence of a ligand. Ligand binding to the extracellular portion of RTK, dimerizes monomeric receptors, resulting in autophosphorylation of specific tyrosine residues in the cytoplasmic portion (for review see: Blume-Jensen, P., and Hunter, T., Nature (2001) 411:355-365; Hubbard, S. R., et al., J. Biol. Chem. 273 (1998) 11987-11990; Zwick, E., et al., Trends Mol. Med. (2002) 8:17-23). In general, tyrosine autophosphorylation either stimulates the intrinsic catalytic kinase activity of the receptor or generates recruitment sites for downstream signaling proteins containing phosphotyrosine-recognition domains, such as the Src homology 2 (SH2) domain or the phosphotyrosine-binding (PTB) domain.

PTK have become primary targets for the development of novel therapeutics designed to block cancer cell proliferation, metastasis, and angiogenesis and promote apoptosis. The strategy that has progressed farthest in clinical development is the use of monoclonal antibodies to target growth factor receptor tyrosine kinases. The use of small molecule tyrosine kinase inhibitors however could have significant theoretical advantages over monoclonal antibodies. Small molecule inhibitors could have better tissue penetration, could have activity against intracellular targets and mutated targets and could be designed to have oral bioavailability. Several lead compounds have shown promising activity against such targets as the EGFR, the vascular endothelial cell growth factor receptor and bcr-abl. The hepatocyte growth factor receptor c-Met was first identified as an activated oncogene in an N-methyl-N′-nitrosoguanidinic treated human osteogenic sarcoma cell line (MUNG-HOS) by its ability to transform NIH 3T3 mouse fibroblasts. The receptor encoded by the c-Met protooncogene (located on chromosome 7) is a two-chain protein composed of 50 kDa (alpha) chain disulfide linked to a 145 kDa (beta) chain in an alpha-beta complex of 190 kDa. The alpha-chain is exposed at the cell surface while the beta chain spans the cell membrane and possesses an intracellular tyrosine kinase domain. The presence of this intracellular tyrosine kinase domain groups c-Met as a member of the receptor tyrosine kinase (RTK) family of cell surface molecules.

Much evidence supports the role of HGF as a regulator of carcinogenesis, cancer invasion and metastasis (for review see: Herynk, M. H., and Radinsky, R. (2000) In Vivo 14:587-596; Jiang et al. (1999) Crit. Rev. Oncol. Hematol. 29:209-248; Longati (2001) Cum Drug Targets 2:41-55; Maulik et al., (2002) Cytokine Growth Factor Rev. 13:41-59; Parr, C., and Jiang, W. G., (2001) Histol. Histopathol. 16:251-268). HGF binds to and induces tyrosine phosphorylation of the mature c-met receptor beta-chain. Such events are thought to promote binding of intracellular signaling proteins containing src homology (SH) regions such as PLC-gamma, Ras-GAP, PI-3 kinase pp⁶⁰c-src and the GRB-2 Socs complex to the activated receptor. Each SH2-containing protein may activate a different subset of signaling phosphopeptides, thus eliciting different responses within the cell. c-Met mutations have been well-described in hereditary and sporadic human papillary renal carcinomas and have been reported in ovarian cancer, childhood hepatocellular carcinoma, metastatic head and neck squamous cell carcinomas, and gastric cancer. c-Met is also over-expressed in both non-small cell lung cancer and small cell lung cancer cells, in lung, breast, colon and prostate tumors (Herynk et al. (2003) Cancer Res. 63(11):2990-2996; Maulik et al. (2002) Clin. Cancer Res. 8:620-627). Since c-Met appears to play an important role in oncogenesis of a variety of tumors, various inhibition strategies have been employed to therapeutically target this receptor tyrosine kinase. The usefulness of inhibiting the protein-tyrosine kinase c-Met for inhibiting tumor growth and invasion has been shown in many well documented preclinical experiments (Abounader et al. (1999) J. Natl. Cancer Inst. 91:1548-1556; Laterra et al. (1997) Lab. Invest. 76:565-577; Tomioka, D. (2001) Cancer Res. 61:7518-7524; Wang et al. (2001) J. Cell Biology 153:1023-1033).

c-Met inhibitors have been reported (US 2004/0242603; US 2004/0110758; US 2005/0009845; WO 2003/000660; WO 98/007695; U.S. Pat. No. 5,792,783; U.S. Pat. No. 5,834,504; U.S. Pat. No. 5,880,141; US 2003/0125370; U.S. Pat. No. 6,599,902; WO 2005/030140; WO 2005/070891; US 2004/0198750; U.S. Pat. No. 6,790,852; WO 2003/087026; U.S. Pat. No. 6,790,852; WO 2003/097641; U.S. Pat. No. 6,297,238; WO 2005/005378; WO 2004/076412; WO 2005/004808; WO 2005/010005; US 2005/0009840; WO 2005/121125; WO 2006/014325). PHA-665752 is a small molecule, ATP-competitive, active-site inhibitor of the catalytic activity of c-Met, as well as phenotypes such as cell growth, cell motility, invasion, and morphology of a variety of tumor cells (Ma et al. (2005) Clin. Cancer Res. 11:2312-2319; Christensen et al. (2003) Cancer Res. 63:7345-7355).

### SUMMARY OF THE INVENTION

In one aspect, the invention relates to heterobicyclic pyrazole compounds that are inhibitors of receptor tyrosine kinases (RTK), including c-Met. Certain hyperproliferative disorders are characterized by the overactivation of c-Met kinase function, for example by mutations or overexpression of the protein. Accordingly, the compounds of the invention are useful in the treatment of hyperproliferative disorders such as cancer.

More specifically, one aspect of the invention provides heterobicyclic pyrazole compounds of Formulas Ia and Ib:

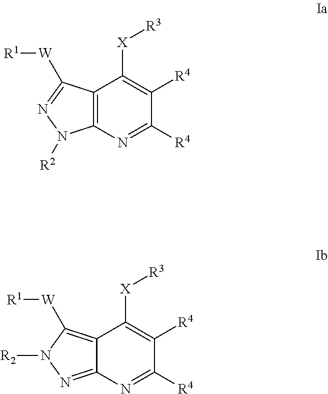

and stereoisomers, geometric isomers, tautomers, solvates, metabolites and pharmaceutically acceptable salts and prodrugs thereof, wherein R¹, R², R³, R⁴, X and Z are as defined herein.

Another aspect of the invention provides a pharmaceutical composition comprising a heterobicyclic pyrazole compound of Formulas Ia or Ib and a pharmaceutically acceptable carrier. The pharmaceutical composition may further comprise one or more additional therapeutic agents selected from anti-proliferative agents, anti-inflammatory agents, immunomodulatory agents, neurotropic factors, agents for treating cardiovascular disease, agents for treating liver disease, anti-viral agents, agents for treating blood disorders, agents for treating diabetes, and agents for treating immunodeficiency disorders.

Another aspect of the invention provides methods of inhibiting or modulating receptor tyrosine kinase activity, comprising contacting the kinase with an effective inhibitory amount of a compound of Formula Ia or Ib.

Another aspect of the invention provides methods of inhibiting c-Met kinase activity, comprising contacting a c-Met kinase with an effective inhibitory amount of a compound of Formula Ia or Ib, or a stereoisomer, geometric isomer, tautomer, solvate, metabolite, or pharmaceutically acceptable salt or prodrug thereof.

Another aspect of the invention provides methods of preventing or treating a disease or disorder modulated by c-Met kinases, comprising administering to a mammal in need of such treatment an effective amount of a compound of Formula Ia or Ib, or a stereoisomer, geometric isomer, tautomer, solvate, metabolite, or pharmaceutically acceptable salt or prodrug thereof. Examples of such diseases, conditions and disorders include, but are not limited to, hyperproliferative disorders (e.g., cancer, including melanoma and other cancers of the skin), neurodegeneration, cardiac hypertrophy, pain, migraine, neurotraumatic diseases, stroke, diabetes, hepatomegaly, cardiovascular disease, Alzheimer's disease, cystic fibrosis, viral diseases, autoimmune diseases, atherosclerosis, restenosis, psoriasis, allergic disorders, inflammation, neurological disorders, hormone-related diseases, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders.

Another aspect of the invention provides methods of preventing or treating a hyperproliferative disorder, comprising administering to a mammal in need of such treatment an effective amount of a compound of Formula Ia or Ib, or a stereoisomer, geometric isomer, tautomer, solvate, metabolite, or pharmaceutically acceptable salt or prodrug thereof, alone or in combination with one or more additional compounds having anti-hyperproliferative properties.

In a further aspect the present invention provides a method of using a compound of this invention to treat a disease or condition modulated by c-Met in a mammal.

An additional aspect of the invention is the use of a compound of this invention in the preparation of a medicament for the treatment or prevention of a disease or condition modulated by c-Met in a mammal.

Another aspect of the invention includes kits comprising a compound of Formula Ia or Ib, or a stereoisomer, geometric isomer, tautomer, solvate, metabolite, or pharmaceutically acceptable salt or prodrug thereof, a container, and optionally a package insert or label indicating a treatment.

Another aspect of the invention includes methods of preparing, methods of separating, and methods of purifying compounds of Formula Ia and Ib.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### Definitions

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂ CH₂CH₃), 2-pentyl(-CH(CH₃)CH₂CH₂CH₃), 3-pentyl(-CH(CH₂CH₃)₂), 2-methyl-2-butyl(-C(CH₃)₂CH₂CH₃), 3-methyl-2-butyl(-CH(CH₃)CH(CH₃)₂), 3-methyl-1-butyl(-CH₂CH₂CH(CH₃)₂), 2-methyl-1-butyl(-CH₂CH(CH₃)CH₂CH₃), 1-hexyl(-CH₂CH₂CH₂CH₂CH₂CH₃), 2-hexyl(-CH(CH₃)CH₂CH₂ CH₂CH₃), 3-hexyl(-CH(CH₂CH₃)(CH₂CH₂CH₃)), 2-methyl-2-pentyl(—C(CH₃)₂CH₂CH₂CH₃), 3-methyl-2-pentyl(-CH(CH₃)CH(CH₃)CH₂CH₃), 4-methyl-2-pentyl(-CH(CH₃)CH₂CH(CH₃)₂), 3-methyl-3-pentyl(-C(CH₃)(CH₂CH₃)₂), 2-methyl-3-pentyl(-CH(CH₂CH₃)CH(CH₃)₂), 2,3-dimethyl-2-butyl(-C(CH₃)₂CH(CH₃)₂), 3,3-dimethyl-2-butyl(-CH(CH₃)C(CH₃)₃, 1-heptyl, 1-octyl, and the like.

The term “alkyl” includes saturated linear or branched-chain monovalent hydrocarbon radicals of one to six carbon atoms (e.g., C₁-C₆ alkyl), wherein the alkyl radical may be optionally substituted independently with one or more substituents described below.

The term “C₁-C₆ fluoroalkyl” includes an alkyl group of 1-6 carbons substituted with a fluoro group. The fluoro group can be substituted at any place on the alkyl group. Examples include, but are not limited to, CH₂F, CH₂CH₂F, CH₂CH₂CH₂F, CH₂CH₂CH₂CH₂F, CH₂CH₂CH₂CH₂CH₂F, and the like.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl propynyl (propargyl, —CH₂C≡CH), and the like.

The terms “carbocycle”, “carbocyclyl”, “carbocyclic ring” and “cycloalkyl” refer to a monovalent non-aromatic, saturated or partially unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and bicyclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Aryl includes bicyclic radicals comprising an aromatic ring fused to a saturated, partially unsaturated ring, or aromatic carbocyclic or heterocyclic ring. Typical aryl groups include, but are not limited to, radicals derived from benzene, substituted benzenes, naphthalene, anthracene, biphenyl, indenyl, indanyl, 1,2-dihydronapthalene, 1,2,3,4-tetrahydronapthyl, and the like.

The terms “heterocycle,” “hetercyclyl” and “heterocyclic ring” are used interchangeably herein and refer to a saturated or a partially unsaturated (i.e., having one or more double and/or triple bonds within the ring) carbocyclic radical of 3 to 20 ring atoms in which at least one ring atom is a heteroatom selected from nitrogen, oxygen and sulfur, the remaining ring atoms being C, where one or more ring atoms is optionally substituted independently with one or more substituents described below. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. The heterocyclyl may be a carbon radical or heteroatom radical. The term “heterocycle” includes heterocycloalkoxy. “Heterocyclyl” also includes radicals where heterocycle radicals are fused with a saturated, partially unsaturated ring, or aromatic carbocyclic or heterocyclic ring. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Examples of a heterocyclic group wherein 2 ring carbon atoms are substituted with oxo (═O) moieties are pyrimidinonyl and 1,1-dioxo-thiomorpholinyl. The heterocycle groups herein are optionally substituted independently with one or more substituents described herein.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The heterocycle or heteroaryl groups may be C-attached or N-attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

“Substituted alkyl”, “substituted alkenyl”, “substituted alkynyl”, “substituted aryl”, “substituted heteroaryl”, “substituted heterocyclyl” and “substituted cycloalkyl” mean alkyl, alkenyl, alkynyl, aryl, heteroaryl, heterocyclyl and cycloalkyl, respectively, in which one or more hydrogen atoms are each independently replaced with a substituent. Typical substituents include, but are not limited to, F, Cl, Br, I, CN, CF₃, OR, R, ═O, ═S, ═NR, ═N⁺(O)(R), ═N(OR), ═N⁺(O)(OR), ═N—NRR′, —C(═O)R, —C(═O)OR, —C(═O)NRR′, —NRR′, —N⁺RR′R″, —N(R)C(═O)R′, —N(R)C(═O)OR′, —N(R)C(═O)NR′R″, —SR, —OC(═O)R, —OC(═O)OR, —OC(═O)NRR′, —OS(O)₂(OR), —OP(═O)(OR)(OR′), —OP(OR)(OR′), —P(═O)(OR)(OR′), —P(═O)(OR)NR′R″, —S(O)R, —S(O)₂R, —S(O)₂NR, —S(O)(OR), —S(O)₂(OR), —SC(═O)R, —SC(═O)OR, ═O and —SC(═O)NRR′; wherein each R, R′ and R″ is independently selected from H, C₁-C₁₂ alkyl, C₂-C₈ alkenyl, C₂-C₈ alkynyl, C₆-C₂₀ aryl and C₂-C₂₀ heterocyclyl. Substituents may also be combinations of alkyl, alkenyl, alkynyl, carbocycle, aryl, and heteroaryl radicals, such as cyclopropylmethyl, cyclohexylethyl, benzyl, and N-ethylmorpholino, and substituted forms thereof.

The terms “treat” and “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTONO (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUICIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

The term “prodrug” as used in this application refers to a precursor or derivative form of a compound of the invention that is less cytotoxic to cells compared to the parent compound or drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs; optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, enzymatic cleavage, and the like, of the administered compound. Accordingly, the invention includes metabolites of compounds of the invention, including compounds produced by a process comprising contacting a compound of this invention with a mammal for a period of time sufficient to yield a metabolic product thereof.

A “liposome” is a small vesicle composed of various types of lipids, phospholipids and/or surfactant which is useful for delivery of a drug (such as the cMet inhibitors disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The phrase “pharmaceutically acceptable salt,” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The compounds of Formulas Ia and Ib also include other salts of such compounds which are not necessarily pharmaceutically acceptable salts, and which may be useful as intermediates for preparing and/or purifying compounds of Formulas Ia or Ib and/or for separating enantiomers of compounds of Formulas Ia or Ib.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition must be compatible chemically and/or toxicologically, with the other ingredients comprising a formulation, and/or the mammal being treated therewith.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilypethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The terms “compound of this invention,” and “compounds of the present invention” and “compounds of Formula Ia and Ib” include compounds of Formulas Ia and Ib and stereoisomers, geometric isomers, tautomers, solvates, metabolites, and pharmaceutically acceptable salts and prodrugs thereof.

The term “mammal” includes, but is not limited to, humans, dogs, cats, horses, cows, pigs, sheep, and poultry.

### C-Met Inhibitor Compounds

The present invention provides heterobicyclic pyrazole compounds, and pharmaceutical formulations thereof, that are potentially useful in the treatment of diseases, conditions and/or disorders modulated by c-Met. More specifically, the present invention provides compounds of Formulas Ia and Ib

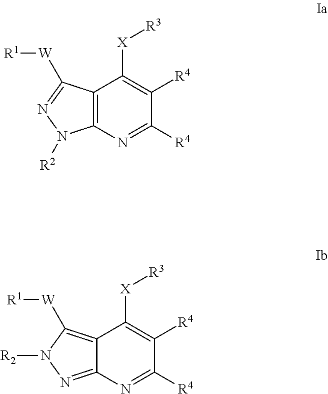

and stereoisomers, geometric isomers, tautomers, solvates, metabolites and pharmaceutically acceptable salts and prodrugs thereof, wherein:

X is O, S or NR¹⁰;

W is O, S, S(═O) or S(═O)₂;

R¹ is H, C₁-C₁₂ alkyl, C₂-C₈ alkenyl, C₂-C₈ alkynyl, —C(═O)NR¹⁰R¹¹, or —(CR¹⁴R¹⁵)NR¹⁰R¹¹, or

R¹ is C₃-C₁₂ carbocyclyl, C₂-C₂₀ heterocyclyl, C₆-C₂₀ aryl, C₁-C₂₀ heteroaryl, (CR¹⁴R¹⁵)ₙC₃-C₁₂ carbocyclyl, (CR¹⁴R¹⁵)ₙC₂-C₂₀ heterocyclyl, (CR¹⁴R¹⁵)ₙC₆-C₂₀ aryl or (CR¹⁴R¹⁵)ₙC₁-C₂₀ heteroaryl, wherein said alkyl, alkenyl, alkynyl, carbocyclyl, heterocyclyl, aryl nd heteroaryl are optionally substituted with one or more groups independently selected from R¹⁰, F, Cl, Br, I, CN, CF₃, oxo, —OR¹⁰, SR¹⁰, —C(═Y)R¹⁰, —C(═Y)OR¹⁰, —C(═Y)NR¹⁰R¹¹, —(CR^{I4}R¹⁵)ₙ—NR¹⁰R¹¹, —NR¹⁰C(═Y)R¹³, —NR¹⁰C(═Y)OR¹¹, —NR¹²C(═Y)NR¹⁰R¹¹, —NR¹²SO₂R¹⁰, —OC(═Y)R¹⁰, —OC(═Y)OR¹⁰, —OC(═Y)NR¹⁰R¹¹, —OS(O)₂(OR¹⁰), OP(═Y)(OR¹⁰)(OR¹¹), —OP(OR¹⁰(OR¹¹), —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, (C₁-C₆ alkyl)OH, —(CH₂)ₙCH(OH)(CH₂)ₘOH, C₁-C₆ fluoroalkyl, C₂-C₈ alkenyl, C₂-C₈ alkynyl, C₃-C₁₂ cycloalkyl, C₂-C₂₀ heterocyclyl, —(CR¹⁴R¹⁵)ₙC₂-C₂₀ heterocyclyl, C₆-C₂₀ aryl, —(CR¹⁴R¹⁵)ₙC₆-C₂₀ aryl, C₁-C₂₀ heteroaryl, —(CR¹⁴R¹⁵)ₜNR¹⁰R¹¹, —NR¹⁰(CR¹⁰R¹¹)ₙCHR¹⁰R¹¹, —(CR¹⁴R¹⁵)—NR¹²C(═O)(CR¹⁴R¹⁵)NR¹⁰R¹¹, —(CR¹⁴R¹⁵)ₜNR¹⁰R¹¹, —C(═Y)(CR¹⁰R¹¹)ₙ—OR¹⁰, and —C(═Y)(CR¹⁰R¹¹)ₙNR¹⁰R¹¹, or

R¹ is NR^{x}R^{y};

R² is H, CF₃, CN, —C(═Y)R¹⁰, —C(═Y)OR¹⁰, —C(═Y)NR¹⁰R¹¹, —C(═O)NR¹²(CR¹⁴R¹⁵)ₜNR¹⁰R¹¹, —SR¹⁰, —S(O)R¹⁰, —S(O)₂R¹⁰, S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C_{I}—Cₘ heteroaryl, wherein said alkyl, alkenyl, alkynyl, carbocyclyl, heterocyclyl, aryl, and heteroaryl are optionally substituted with one or more groups independently selected from F, Cl, Br, I, (CH₂)ₙOR¹⁰, (CH₂)ₙNR¹⁰R¹¹, heteroaryl and heterocyclyl;

R³ is C₃-C₁₂ carbocyclyl, C₂-C₂₀ heterocyclyl, C₆-C₂₀ aryl or C₁-C₂₀ heteroaryl, wherein said carbocyclyl, heterocyclyl, aryl and heteroaryl are optionally substituted with one or more groups independently selected from F, Cl, Br, I, CN, CF₃, OR¹⁰, SR¹⁰, —C(═Y)R¹⁰, —C(═Y)OR¹⁰, —C(═Y)NR¹⁰R¹¹, —NR¹⁰R¹¹, —NR¹⁰C(═Y)R¹³, —NR¹⁰C(═Y)OR¹¹, —NR¹²C(═Y)NR¹⁰R¹¹, —NR¹²C(═O)C(═O)R¹⁰R¹¹, —NR¹²C(═O)C(═O)OR^{a}, —NR¹²SO₂R¹⁰, —NR¹²C(═Y¹)(CR¹⁴R¹⁵)ₙC(═Y²)NR¹⁰R¹¹, —NR¹²C(═Y¹)NR¹⁰C(═Y²)(CR¹⁴R¹⁵)ₙR¹¹, —NR¹²C(═Y¹)(cR¹⁴R¹⁵)ₙC(═Y²)(CR¹⁴R¹⁵)ₘR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl, wherein said alkyl, alkenyl, alkynyl, cycloalkyl, heterocyclyl, aryl and heteroaryl are optionally substituted with one or more groups independently selected from F, Cl, Br, I, OH, C₁-C₁₂ alkyl, NR¹⁰R¹¹, and (CR¹⁴R¹⁵)ₙ-aryl;

R⁴ is H, F, Cl, Br, CF₃, CN, —C(═Y)R¹⁰, —C(═Y)OR¹⁰, —C(═Y)NR¹⁰R¹¹, —NR¹⁰R¹¹, NR¹⁰C(═Y)R¹¹, NR¹⁰C(═Y)OR¹¹, NR¹²C(═Y)NR¹⁰R¹¹, —NR¹²SO₂NR¹⁰R¹¹, —OR¹⁰, —OC(═Y)R¹⁰, OC(═Y)OR¹⁰, —OC(═Y)NR¹⁰R¹¹, —C(═Y)NR¹²(CR¹⁴R¹⁵)ₜNR¹⁰R¹¹, —OP(═Y)(OR¹⁰)(OR¹¹), —OP(OR¹⁰)(OR¹¹), —SR¹⁰, —S(O)R¹⁰, —S(O)₂R¹⁰, —S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;

R¹⁰, R¹¹ and R¹² are independently H, C₁-C₁₂ alkyl, C₂-C₈ alkenyl, C₂-C₈ alkynyl, OR^{a}, NR^{a}R^{b}, C₃-C₁₂ carbocyclyl, (CR¹⁴R¹⁵)ₙC₂-C₂₀ heterocyclyl, (CR¹⁴R¹⁵)ₙC₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₃-C₁₂ carbocyclyl, C₆-C₂₀ aryl, and C₁-C₂₀ heteroaryl,

or R¹⁰ and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₙOR^{a}, NR^{a}R^{b}, CF₃, F, Cl, Br, I, SO₂R^{a}, C(═O)R^{a}, NR¹⁰C(═Y)R¹¹, C(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl,

or R¹⁰ and R¹² together with the atoms to which they are attached form an oxo-substituted C₃-C₂₀ heterocyclic ring optionally fused to a benzene ring;

R¹³ is H, C₁-C₁₂ alkyl, C₂-C₈ alkenyl, C₂-C₈ alkynyl, (CR¹⁴R¹⁵)ₙ-cycloalkyl, (CR¹⁴R¹⁵)ₙ-heterocyclyl, (CR¹⁴R¹⁵)ₙ-aryl, (CR¹⁴R¹⁵)ₙ-heteroaryl, (CR¹⁴R¹⁵)ₙ—O—(CR¹⁴R¹⁵)ₘ-aryl, (CR¹⁴R¹⁵)ₙ—OR¹⁰, (CR¹⁴R¹⁵)ₙ—NR¹⁰R¹¹, (CR¹⁴R¹⁵)ₙ—NR¹⁰C(═O)R¹¹, or (CR¹⁴R¹⁵)ₙ—NR¹⁰(SO₂Me)-R¹¹, wherein said alkyl, alkenyl, alkynyl, cycloalkyl, heterocycloalkyl, and heteroaryl portions are optionally substituted with one or more groups independently selected from F, Cl, Br, I, oxo, SO₂R^{c}, CN, OR^{a}, C(═O)R^{a}, C(═O)OR^{a}, NR^{a}R^{b}, NR^{a}C(═O)R^{b}, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl, and C₁-C₂₀ heteroaryl, wherein said alkyl, C₂-C₈ alkenyl, C₂-C₈ alkynyl, C₃-C₁₂ cycloalkyl, C₂-C₂₀ heterocyclyl, C₆-C₂₀ aryl, and C₁-C₂₀ heteroaryl are optionally substituted with one or more groups independently selected from F, Cl, Br, and I;

each R¹⁴ and R¹⁵ is independently H, C₁-C₁₂ alkyl, or (CH₂)ₜ-aryl,

or R¹⁴ and R¹⁵ together with the atoms to which they are attached form a saturated or partially unsaturated C₃-C₁₂ carbocyclic ring,

or R¹⁰ and R¹⁵ together with the atoms to which they are attached form an oxo-substituted saturated or partially unsaturated monocyclic or bicyclic C₁-C₂₀ heterocyclic ring optionally further substituted with one or more groups independently selected from F, Cl, Br, I, OR^{a}, C₁-C₁₂ alkyl, C₂-C₈ alkenyl, C₂-C₈ alkynyl, C₃-C₁₂ cycloalkyl, C₂-C₂₀ heterocyclyl, C₆-₂₀ aryl, or C₁-C₂₀ heteroaryl, wherein said alkyl and aryl are optionally substituted with one or more groups independently selected from F, Cl, Br, and I,

or R¹⁴ is null and R¹⁰ and R¹⁵ together with the atoms to which they are attached form a C₁-C₂₀ heteroaryl ring having one or more heteroatoms;

R^{a} and R^{b} are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from C₁-C₆ alkyl and halogen;

R^{c} is C₁-C₁₂ alkyl or C₆-C₂₀ aryl, wherein said alkyl and aryl are optionally substituted with one or more groups independently selected from F, Cl, Br, I, OR^{a} and C(═O)NR^{a}R^{b};

R^{x} is H or C₁-C₆ alkyl;

R^{y} is (i) (C₁-C₆ alkyl)NR^{j}R^{k} wherein R^{j} and R^{k} are independently H or C₁-C₆ alkyl; (ii) C₅-C₆ cycloalkyl optionally substituted with OH or —OC(═O)CF₃; or (iii) a 5-6 membered heterocyclic ring having 1 to 2 ring heteroatoms independently selected from N and O and optionally substituted with a halogen group, C₁-C₆ alkyl, (C₁-C₆ alkyl)OH, (C₁-C₆ alkyl)O(C₁-C₆ alkyl), or C₁-C₆ fluoroalkyl;

Y, Y¹ and Y² are independently O or S;

t is 1, 2, 3, 4, 5 or 6; and

n and m are independently 0, 1, 2, 3, 4, 5 or 6.

In certain embodiments, W is O.

In certain embodiments, X is O.

In certain embodiments, X is S.

In certain embodiments, X is NR¹⁰. In certain embodiments, R¹⁰ is C₁-C₆ alkyl. In certain embodiments, X is NH.

In certain embodiments, X is NR¹⁰. In certain embodiments, R¹⁰ is (CR¹⁴R¹⁵)ₙC₂-C₂₀ heterocyclyl. In certain embodiments, R¹⁴ and R¹⁵ are hydrogen. In certain embodiments n is 2. In certain embodiments R¹⁰ is (CH₂CH₂)C₄ heterocyclyl. In certain embodiments, the heterocyclyl is a morpholinyl group.

Exemplary embodiments of X include the following structures:

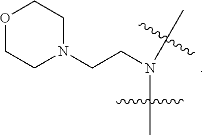

wherein the wavy lines indicate the points of attachment to the pyrazolo[3,4-b]pyridine and R³.

Formula Ia and Ib compounds are regioisomers, differing by the attachment of R² at the non-equivalent nitrogen atoms of the pyrazole ring. Exemplary embodiments of Formula Ia and Ib compounds include, but are not limited to, the following structures:

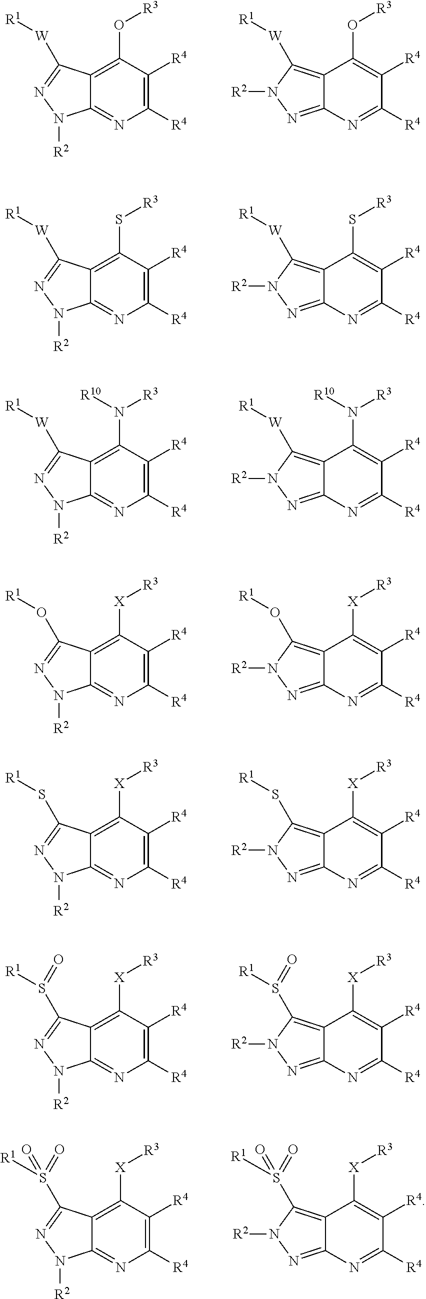

In certain embodiments, R² is H, C₁-C₄ alkyl, CF₃, CHF₂ or CH₂F.

In particular embodiments, R² is C₁-C₆ alkyl or H.

In other embodiments, R² is H.

In certain embodiments, R¹ is H, C₁-C₄ alkyl, CF₃, CHF₂ or CH₂F.

In certain embodiments, R¹ is optionally substituted alkynyl. For example, in certain embodiments R¹ is alkynyl optionally substituted by ≠(CR¹⁴R¹⁵)—NR¹²C(═O)(CR¹⁴R¹⁵)NR¹⁰R¹¹ or —(CR⁴R⁵)ₜNR¹⁰R¹¹ wherein t, R¹⁰, R¹¹, R¹², R¹⁴, and R¹⁵ are as defined herein.

In certain embodiments, t is 1.

In certain embodiments, R¹⁰ is H or C₁-C₆ alkyl.

In certain embodiments, R¹¹ is H or C₁-C₆ alkyl.

In other embodiments, R¹⁰ and R¹¹ together with the nitrogen atom to which they are attached form a 5-6 membered heterocyclic ring optionally having a second ring heteroatom selected from N, O, SO and SO₂ and optionally substituted with one or two groups independently selected from N(C₁-C₆ alkyl)₂, OH, CF₃ and C(═O)(C₁-C₆ alkyl).

In certain embodiments, R¹² is H or C₁-C₆ alkyl.

In certain embodiments, R¹⁴ and R¹⁵ are H or Me.

In certain embodiments, R¹ is an optionally substituted aryl or heteroaryl.

In certain embodiments, R¹ is phenyl optionally substituted with halogen (e.g., F or Cl), C₁-C₆ alkyl, C(═O)C₁-C₆ alkyl, C(═O)(C₃-C₆ cycloalkyl), C(═O)O(C₁-C₆ alkyl), CH₂-heteroaryl (wherein said heteroaryl is a 5 membered ring having 2-3 ring nitrogen atoms), CH₂-hetCyc (wherein hetCyc is a 6 membered ring having 1 to 2 ring heteroatoms independently selected from N and O and optionally substituted with C₁-C₆ alkyl), C(═O)NH(CH₂)₂-hetCyc wherein hetCyc is a 6 membered ring having 1 to 2 ring heteroatoms independently selected from N and O), SO₂NH(C₁-C₆ alkyl), NMeOMe, C(═O)NR^{h}Rⁱ, or NR^{h}Rⁱ wherein R^{h} and Rⁱ are independently H or C₁-C₆ alkyl.

In certain embodiments, R¹ is a phenyl group fused to a 6, 7, or 8 membered azacyclic ring (such as a piperidinyl ring) optionally substituted with oxo.

In certain embodiments, R¹ is a 5-6 membered heteroaryl having a ring heteroatom selected from N and O and optionally substituted with C(═O)NH(C₁-C₆ alkyl) or CH₂-hetCyc wherein hetCyc is a 6 membered azacycle (such as a piperazinyl group) optionally substituted with C₁-C₆ alkyl.

Exemplary embodiments of R¹ include the following structures:

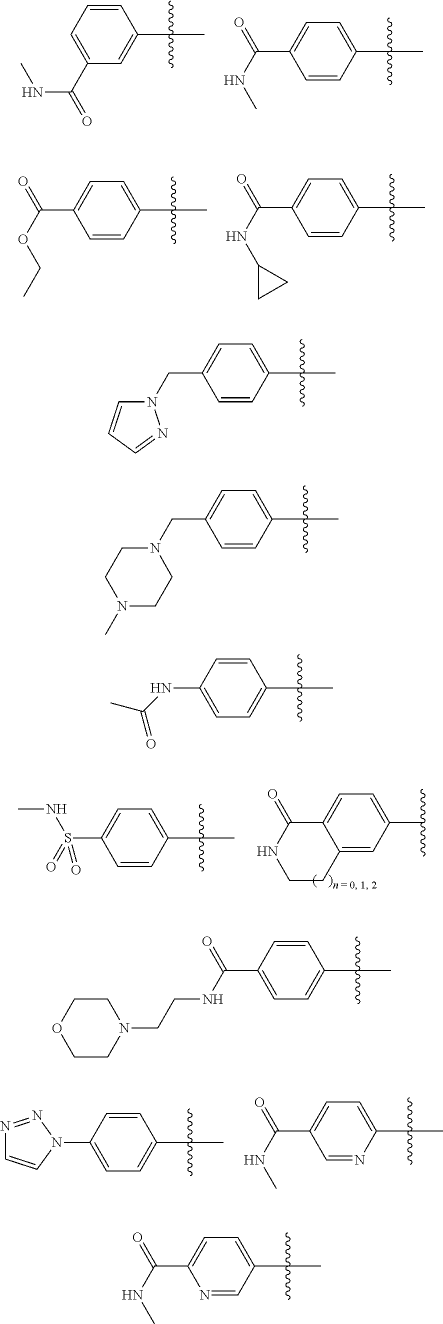

and substituted forms thereof.

Further exemplary embodiments of R¹ include the following structures:

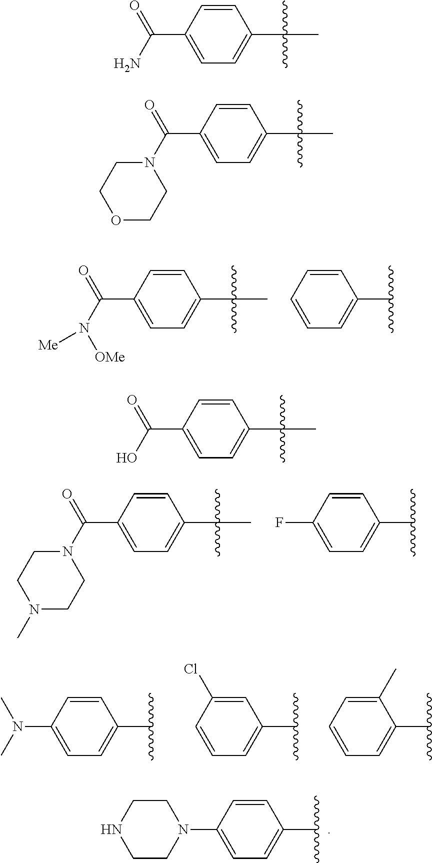

In certain embodiments, R¹ is a 5 membered heteroaryl having at least one N heteroatom and optionally substituted with C₁-C₆ alkyl.

Exemplerary embodiments of R¹ include the following structures:

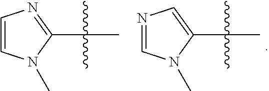

In certain embodiments, R¹ is —C(═O)NR¹⁰R¹¹ or —(CR¹⁴R¹⁵)ₜNR¹⁰R¹¹.

In certain embodiments, R¹⁴ and R¹⁵ are H.

In certain embodiments, R¹⁰ is H or C₁-C₆ alkyl.

In certain embodiments, R¹¹ is C₁-C₆ alkyl or (C₁-C₆ alkyl)OR^{h} wherein R^{h} is H or C₁-C₆ alkyl.

In certain embodiments, R¹⁰ and R^{l1} together with the nitrogen atom to which they are attached form a 6 membered ring optionally having a second ring heteroatom selected from N and O optionally substituted with C₁-C₆ alkyl.

Exemplary embodiments of R¹ include the following structure:

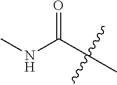

and substituted forms thereof.

Further exemplary embodiments of R¹ include the following structures:

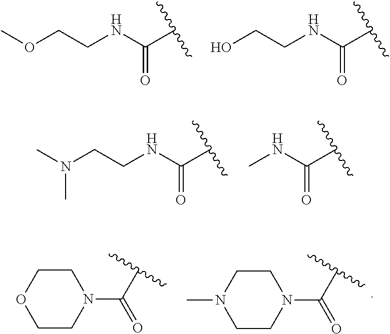

Further exemplary embodiments of R¹ include the following structure:

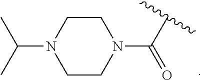

In certain embodiments of compounds of Formula Ia and Ib, R¹ is C₁-C₁₂ alkyl or C₃-C₁₂ carbocyclyl optionally substituted with one or more groups independently selected from OR¹⁰, NR¹⁰R¹¹, NR¹⁰(CR¹⁰R¹¹)ₙCHR¹⁰R¹¹, heterocyclyl and heteroaryl.

In certain embodiments, R¹ is alkyl substituted with a 6 membered heterocyclic group having a ring nitrogen atom and optionally having a second ring heteroatom selected from N and O, wherein said heterocyclic ring is optionally substituted with —O(C₁-C₆ alkyl) or C₁-C₆ alkyl.

In certain embodiments, R¹ is alkyl substituted with a 5 membered heteroaryl group having one or two ring nitrogen heteroatoms.

In certain embodiments, R¹ is alkyl substituted with C₁-C₁₂ alkyl substituted with —(CR¹⁴R¹⁵)ₙ—NR¹⁰R¹¹. In certain embodiments, n is 0. In certain embodiments, R¹⁰ and R¹¹ are hydrogen or C₁-C₁₂ alkyl. In certain embodiments, R¹ is —CH₂CH₂CH₂N(CH₃)₂ or —CH₂CH₂CH₂NH₂.

Exemplary embodiments of R¹ include, but are not limited to, methyl, CH₂OH, CH₂CH₂OH, CH₂CH₂CH₂OH, CH(OH)CH₂OH, CH₂CH(OH)CH₂OH,

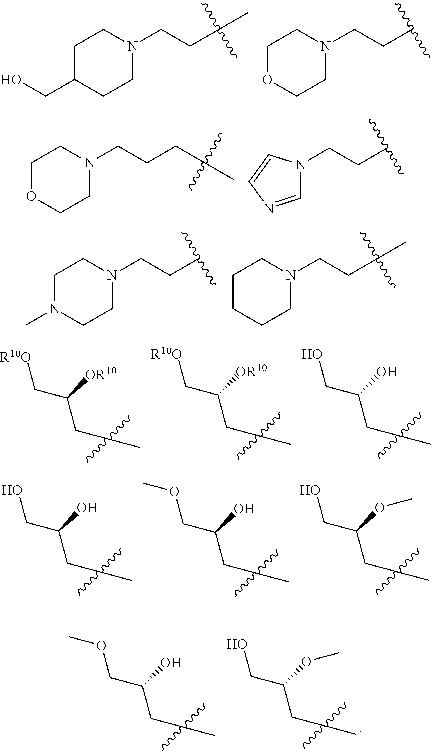

A further exemplary embodiment includes

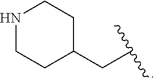

A further exemplary embodiment of R¹ includes the structure:

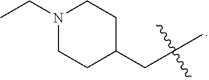

A further exemplary embodiment of R¹ includes the structures:

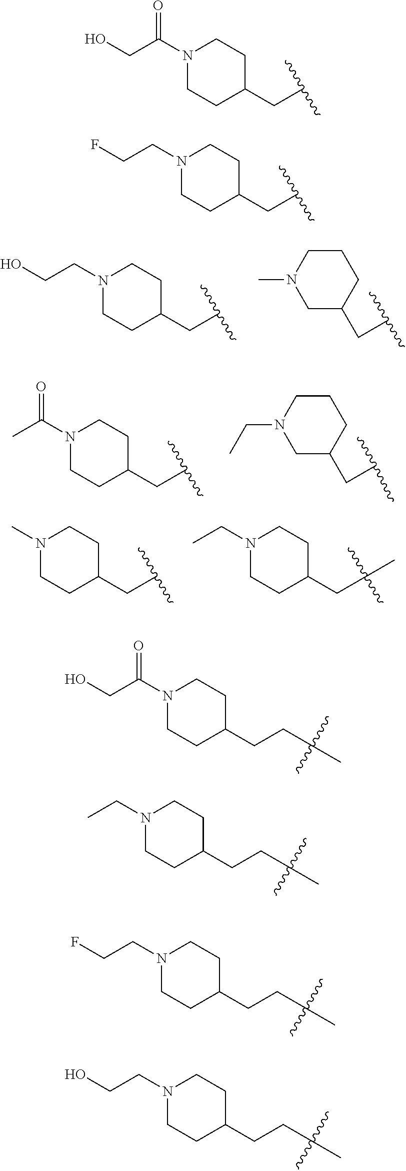

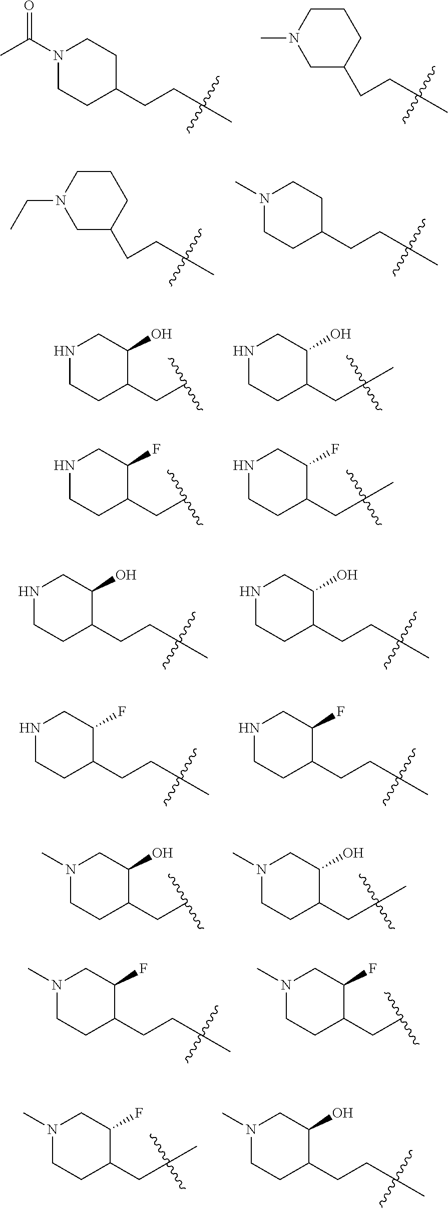

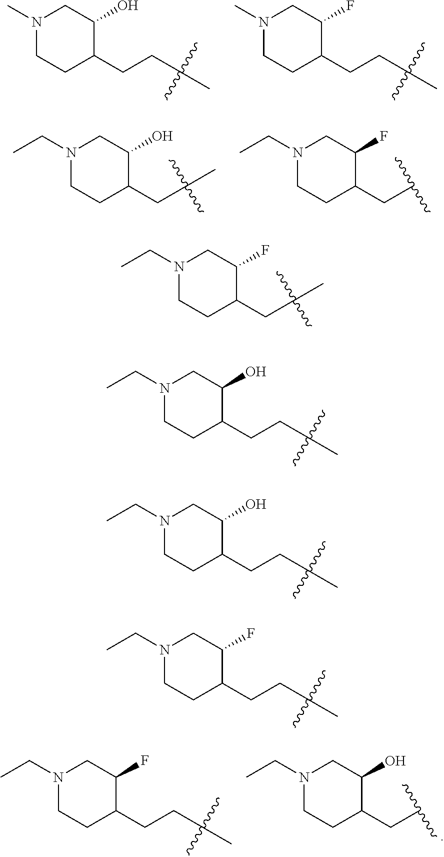

Further exemplary embodiments of R¹ include:

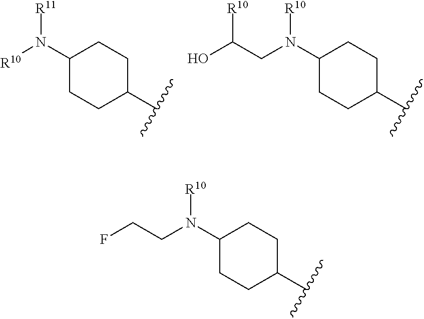

In certain embodiments of compounds of Formula Ia and Ib, R¹ is optionally substituted heteroaryl.

In certain embodiments, R¹ is a 5-6 membered heteroaryl ring having 1 to 2 ring heteroatoms independently selected from N and O and optionally substituted with one or two groups independently selected from R¹⁰, Br, hetCyc and CH₂-hetCyc, wherein hetCyc is a 6 membered heterocyclic ring having a ring nitrogen atom and optionally having a second ring heteroatom selected from N and O, wherein hetCyc is optionally substituted with C₁-C₆ alkyl or (C₁-C₆ alkyl)OH.

Further exemplary embodiments of le include, but are not limited to, the following

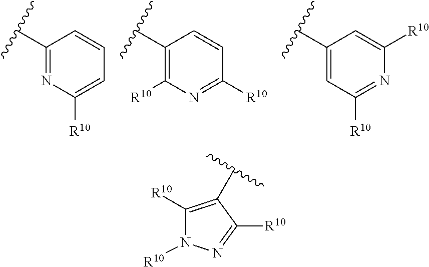

structures:

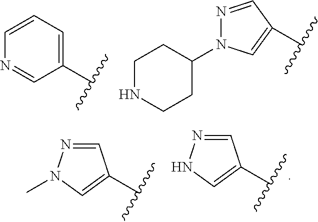

In certain embodiments of compounds of Formula Ia and Ib, R¹ is a saturated or partially unsaturated 5-10 membered monocyclic or bicyclic heterocyclic ring, wherein said ring has one or two ring atoms independently selected from N and O and is optionally substituted with R¹⁰, C₁-C₆ alkyl, (C₁-C₆ alkyl)O(C₁-C₆ alkyl), halo, OR¹⁰, —C(═O)R¹⁰, —C(═O)(CR¹⁰R¹¹)ₙ—OR¹⁰, —C(═O)(CR¹⁰R¹¹)ₙ—NR¹⁰R¹¹, (C₁-C₆ alkyl)OH, C₁-C₆ fluoroalkyl, NR¹⁰R¹¹ or CH₂NR¹⁰R¹¹. Exemplary embodiments of R¹ include, but are not limited to, the following structures:

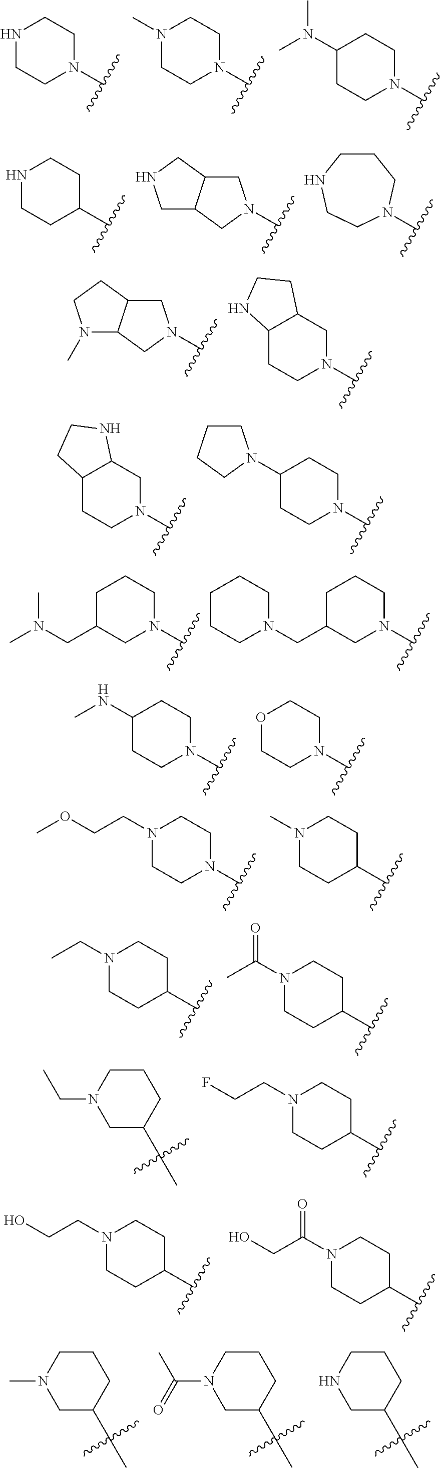

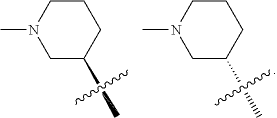

In a further exemplary embodiment, R¹ is a saturated 6-membered monocyclic heterocyclic ring, wherein said ring has one or two ring atoms independently selected from N and O and is optionally substituted with —C(═O)(CR¹⁰R¹¹)ₙ—NR¹⁰R¹¹. In certain embodiments, the heterocyclic ring is a piperidinyl ring. In certain embodiments, the heterocyclic ring is a piperidinyl ring, n is 1 and R¹⁰ and R¹¹ are H or C₁-C₃ alkyl.

In further exemplary embodiments, R¹ includes the structures:

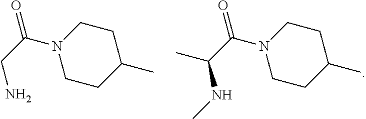

A further exemplary embodiment of R¹ includes the structures:

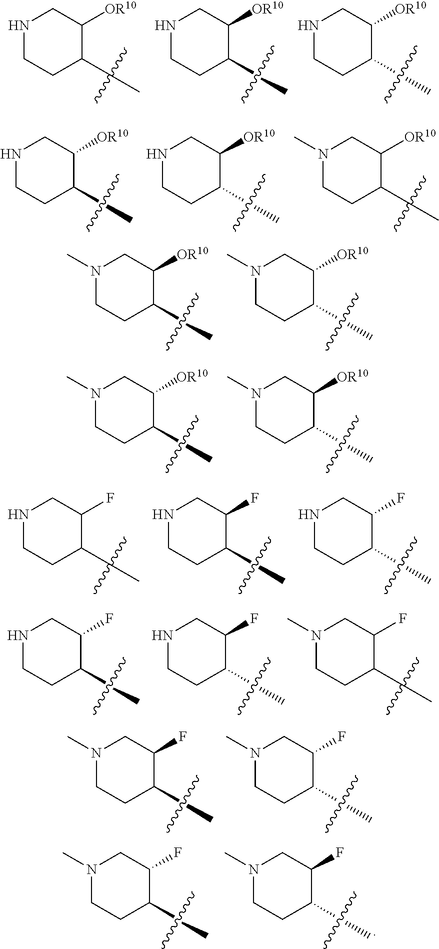

A further exemplary embodiment of R¹ include the structures:

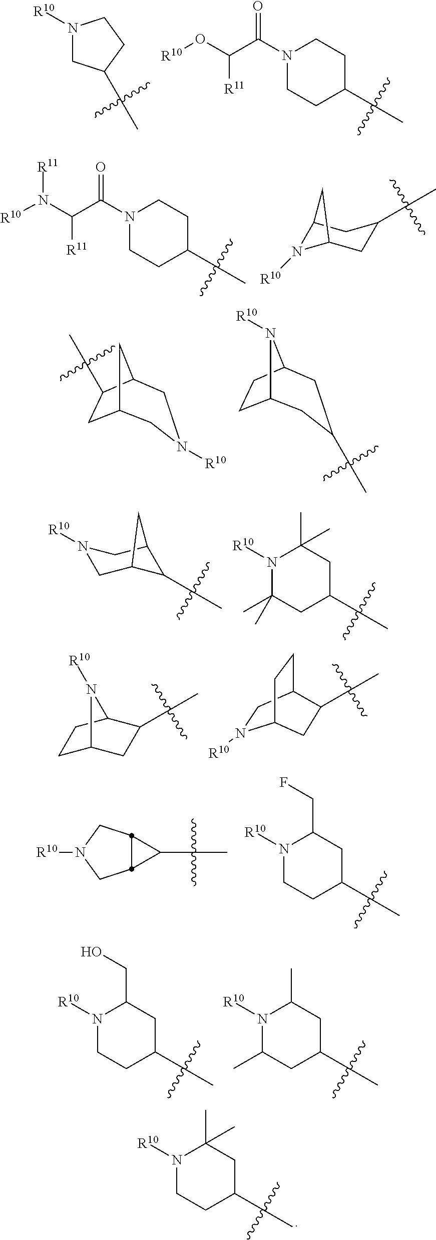

In certain embodiments of compounds of Formula Ia and Ib, R¹ is NR^{x}R^{y}.

In certain, embodiments, R^{x} is H or Me.

In certain embodiments, R^{y} is (i) (C₁-C₆ alkyl)NR^{j}R^{k} wherein R^{j} and R^{k} are independently H or C₁-C₆ alkyl; (ii) cyclohexyl optionally substituted with OH or OC(═O)CF₃; or (iii) a 5-6 membered heterocyclic ring having a ring heteroatom selected from N and O and optionally substituted with F, (C₁-C₆ alkyl), (C₁-C₆ alkyl)OH, (C₁-C₆ alkyl)O(C₁-C₆ alkyl) or (C₁-C₆ fluoroalkyl).

Exemplary embodiments of R¹ include, but are not limited to, the following structures:

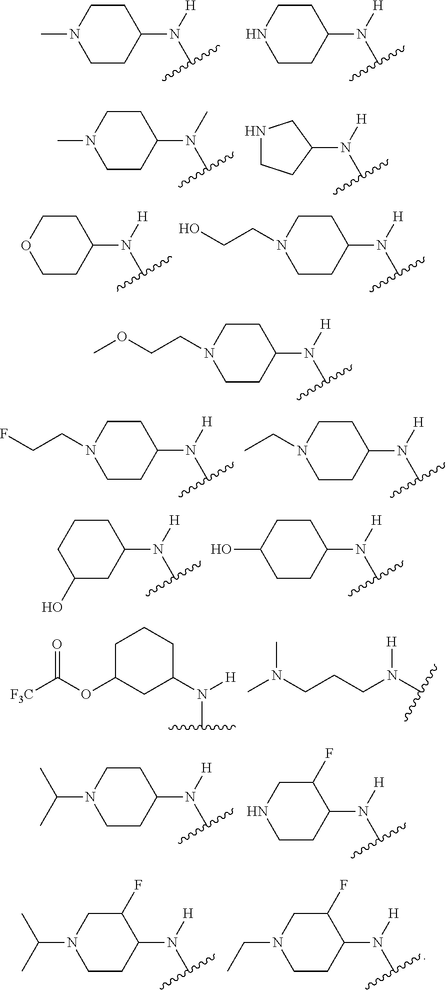

In certain embodiments of compounds of Formula Ia and Ib, R¹ is —(CR¹⁴R¹⁵)NR¹⁰R¹¹. In certain embodiments, t is 0. In certain embodiments, R¹⁰ is H. In certain embodiments R¹¹ is an 8 membered bicyclic heterocyclic ring having a N heteroatom and optionally substituted with C₁-C₆ alkyl.

Exemplary embodiments of R¹ include the structure:

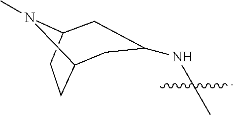

In certain embodiments of compounds of Formula Ia and Ib, R¹ is —C(═Y)OR¹⁰. In certain embodiments, Y is O. In certain embodiments, R¹⁰ is C₁-C₆ alkyl. A particular example is —C(O)OCH₃.

In certain embodiments of compounds of Formula Ia and Ib, R³ has the structure:

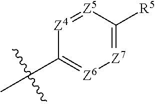

wherein the wavy line indicates the point of attachment to X;

Z⁴, Z⁵, Z⁶, and Z⁷ are independently CR^{4a} or N and 0, 1, or 2 of Z⁴, Z⁵, Z⁶, and Z⁷ is N, wherein when Z⁴ and Z⁵ or Z⁶ and Z⁷ are CR^{4a}, then Z⁴ and Z⁵ or Z⁶ and Z⁷ optionally form a saturated, partially unsaturated or fully unsaturated carbocyclic or heterocyclic ring;

each R^{4a} is independently H, F, Cl, Br, CF₃, CN, —C═Y)R¹⁰, —C(═Y)OR¹⁰, —C(═Y)NR¹⁰R¹¹, —NR¹⁰R¹¹, NR¹⁰C(═Y)R¹¹, NR¹⁰C(═Y)OR¹¹, NR¹²C(═Y)NR¹⁰R¹¹, —NR¹²SO₂NR¹⁰R¹¹, —OR¹⁰, —OC(═Y)R¹⁰, —OC(═Y)OR¹⁰, —OC(═Y)NR¹⁰R¹¹, —C(═O)NR¹²(CR¹⁴R¹⁵)NR¹⁰R¹¹, —OP(═Y)(OR¹⁰)(OR¹¹), —OP(OR¹⁰)(OR¹¹), —SR¹⁰, —S(O)R¹⁰, —S(O)₂R¹⁰, —S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl; and

R⁵ is F, Cl, Br, I, CN, CF₃, OR¹⁰, SR¹⁰, —C(═Y)R¹⁰, —C(═Y)OR¹⁰, —C(═Y)NR¹⁰R¹¹, —NR¹⁰R¹¹, —NR¹⁰C(═Y)R¹³, —NR¹⁰C(═Y)OR¹¹, —NR¹²C(═Y)NR¹⁰R¹¹, —NR¹²C(═O)C(═O)R¹⁰R¹¹, —NR¹²C(═O)C(═O)OR^{a}, —NR¹²SO₂R¹⁰, —NR¹²C(═Y¹)(CR¹⁴R¹⁵)ₙC(═Y²)NR¹⁰R¹¹, —NR¹²C(═Y¹)NR¹⁰C(═Y²)(CR¹⁴R¹⁵)ₙR¹¹, —NR¹²C(═Y¹)(CR¹⁴R¹⁵)ₙC(═Y²)(CR¹⁴R¹⁵)ₘR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl, wherein said alkyl, alkenyl, alkynyl, cycloalkyl, heterocyclyl, aryl and heteroaryl are optionally substituted with one or more groups independently selected from alkyl, NR¹⁰R¹¹, and (CR¹⁴R¹⁵)ₙ-aryl.

In certain embodiments of R³ as defined above, R^{4a} is CH or N.

For example, in certain embodiments of Formula Ia and Ib compounds, R³ is selected from the structures:

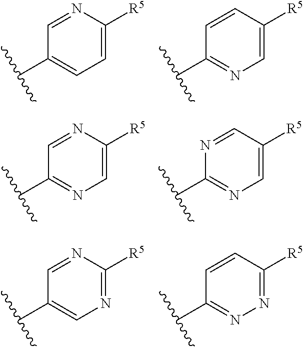

and substituted forms thereof, wherein the wavy line indicates the point of attachment to X, and R⁵ is as defined herein. Exemplary embodiments of R³ include the following structures:

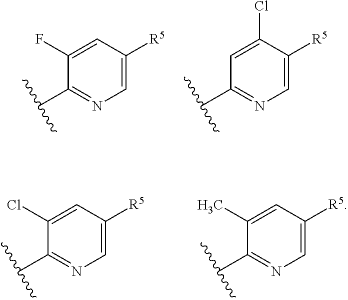

In certain embodiments of compounds of Formula Ia and Ib, R³ is a bicyclic heteroaryl ring substituted with an R⁵ group, wherein R⁵ is as defined above. An exemplary embodiment is the structure:

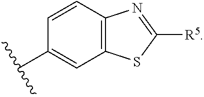

In certain embodiments of Formula Ia and Ib compounds, R³ is selected from the structure:

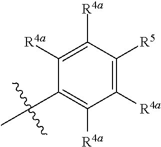

wherein the wavy line indicates the point of attachment to X, and R^{4a} and R⁵ are as defined herein.

In certain embodiments, each R^{4a} is independently selected from H, F, Cl, C₁-C₆ alkyl, O—(C₁-C₆ alkyl), and CN.

Exemplary embodiments of R³ include the following structures:

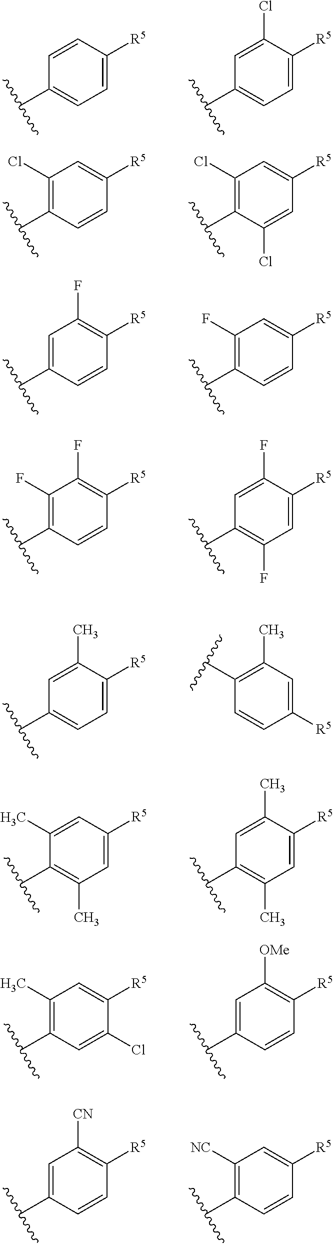

wherein the wavy line indicates the point of attachment to X, and R⁵ is as defined herein.

Additional exemplary embodiments of R³ include the structures:

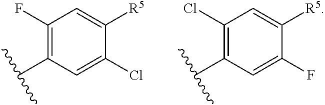

Further exemplary embodiments of Formula Ia and Ib compounds include compounds wherein R³ is

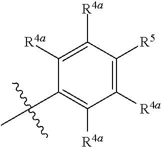

wherein R^{4a} and R⁵ are as defined herein and two adjacent R^{4a} groups together with the atoms to which they are attached form a saturated, partially unsaturated or fully unsaturated carbocyclic or heterocyclic ring. For example, in certain embodiments R³ is selected from the following structures:

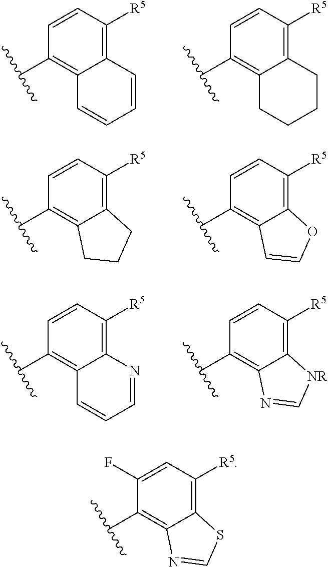

Exemplary embodiments of compounds of Formulas Ia and Ib include the following structures:

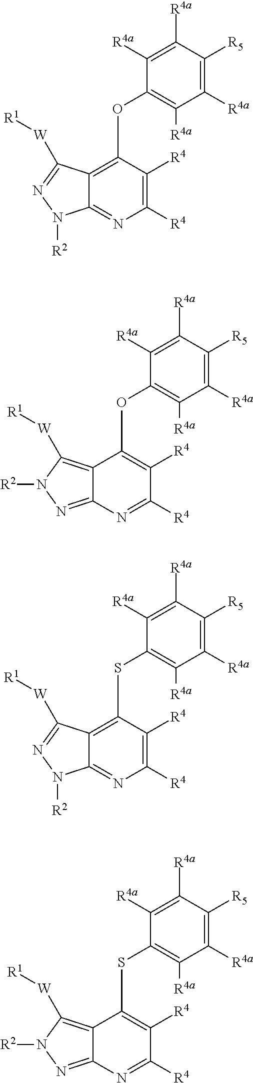

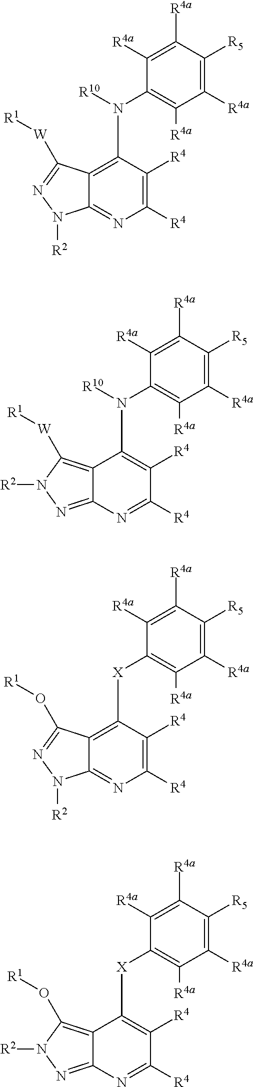

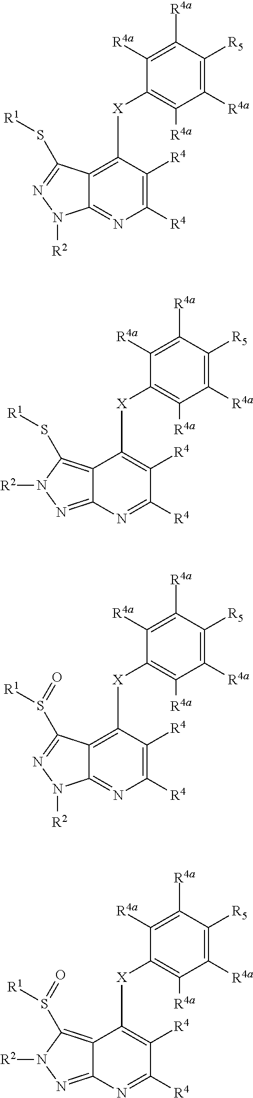

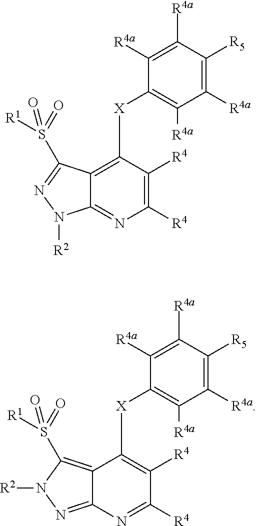

In certain embodiments of compounds of Formulas Ia and Ib, R⁵ has the structure:

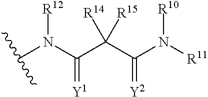

wherein R¹⁰, R¹¹, R¹², R¹⁴, R¹⁵, Y¹ and Y² are as defined herein.

In certain embodiments, Y¹ is O.

In certain embodiments, Y² is O.

In certain embodiments, R¹² is H or C₁-C₆ alkyl.

In certain embodiments, R¹⁴ is H.

In certain embodiments, R¹⁵ is H.

In certain embodiments, R¹⁰ is H.

In certain embodiments, R¹¹ is phenyl optionally substituted with a halogen group.

Exemplary embodiments of R⁵ include the structure

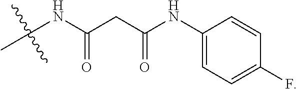

In further exemplary embodiments, R¹⁴ and R¹⁵ together with the atom to which they are attached form an optionally substituted carbocyclic ring. In certain embodiments, R¹⁴ and R¹⁵ together with the carbon atom to which they are attached form a cyclopropylidine group.

For example, in certain embodiments R⁵ is:

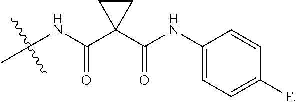

In further exemplary embodiments, R¹⁵ and R¹⁰ together with the atom to which they are attached form an oxo-substituted heterocyclic ring, wherein said heterocyclic ring is optionally further substituted.

In certain embodiments, R¹⁰ and R¹⁵ together with the atoms to which they are attached form an oxo-substituted 5, 6, or 7 membered azacyclic ring.

For examnle, in certain embodiments R⁵ is selected from the structures:

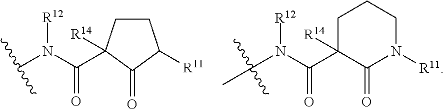

In certain embodiments, R is H.

In certain embodiments, R¹⁴ is H, methyl or benzyl.

In certain embodiments, R¹¹ is H, C₁-C₆ alkyl, or phenyl optionally substituted with one or two groups independently selected from F and Cl.

For example, in certain embodiments R⁵ is selected from the structures:

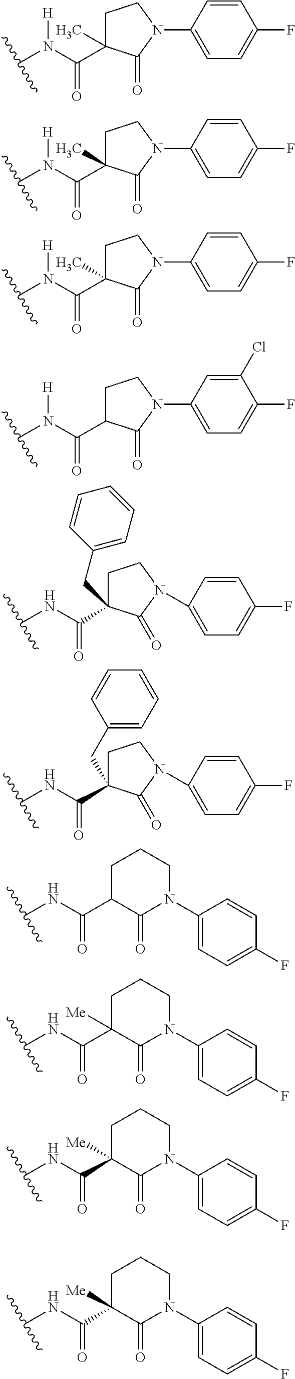

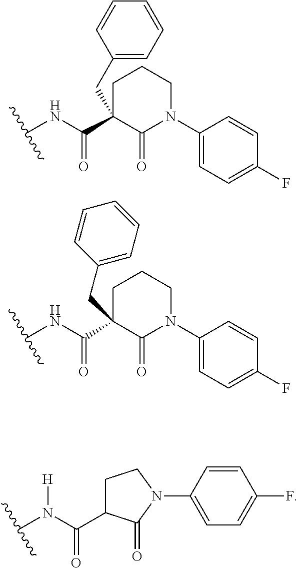

Further exemplary embodiments of R⁵ include the structures:

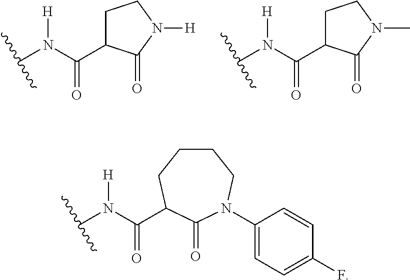

In further exemplary embodiments, R¹⁵ and R¹⁰ together with the atoms to which they are attached form an oxo-substituted bicyclic azacyclic ring, for example an oxo-substituted 6 membered bicyclic azacyclic ring such as an azabicyclo[3.1.0]hexane group. An exemplary embodiment of R⁵ includes the structure:

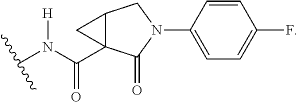

In further exemplary embodiments, R¹⁴ is null and R¹⁰ and R¹⁵ together with the atoms to which they are attached form a heteroaryl ring having a ring nitrogen atom and substituted with ═Y, wherein said heteroaryl ring optionally has one or more additional heteroatoms independently selected from N, O and S

In certain embodiments, R¹⁰ and R¹⁵ together with the atoms to which they are attached form an oxo-substituted 6 membered heteroaryl ring having one or two ring nitrogen atoms.

For example, in certain embodiments R⁵ is selected from the structures:

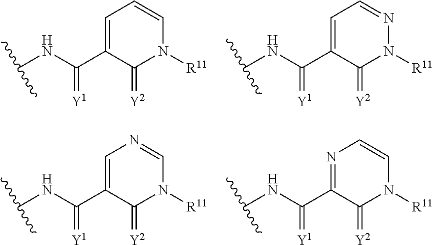

and substituted forms thereof, wherein Y¹, Y² and R¹¹ are as defined herein. In certain embodiments, R¹¹ is optionally substituted aryl, cycloalkyl, or alkyl.

In certain embodiments, Y¹ is O.

In certain embodiments, Y² is O.

In certain embodiments, R¹¹ is phenyl optionally substituted with F.

In certain embodiments, R¹¹ is benzyl.

In certain embodiments, R¹¹ is C₁-C₆ alkyl.

For example, in certain embodiments R⁵ is selected from the structures:

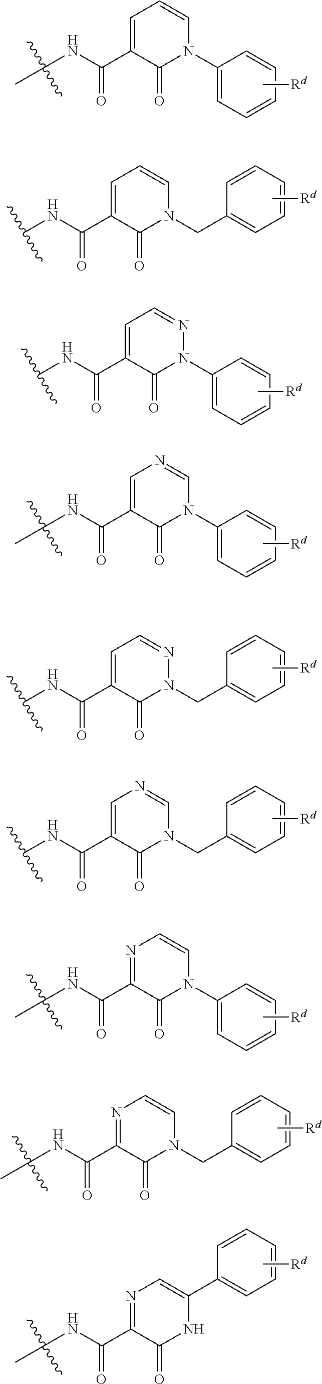

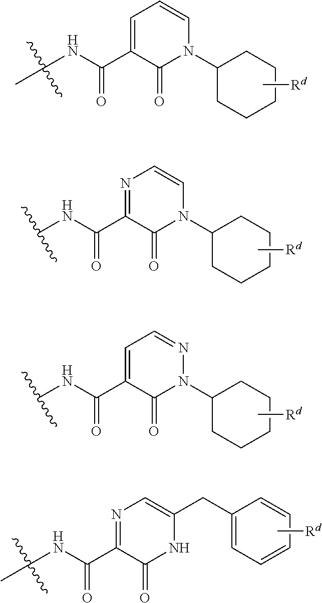

wherein the phenyl and cyclohexyl groups are optionally substituted with one or more R^{d} groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl. In certain embodiments, the phenyl and cyclohexyl groups are optionally substituted with one R^{d} group. In certain embodiments, R^{d} is F.

Exemplary embodiments of R⁵ include the structures:

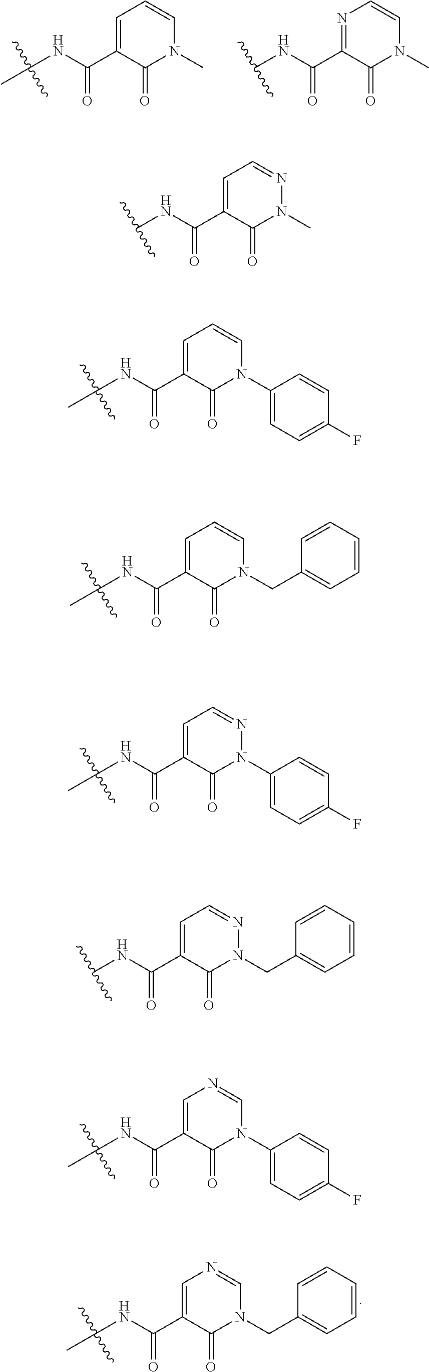

Further exemplary embodiments of R5 include the structure:

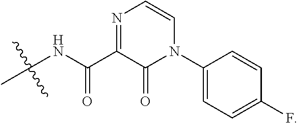

In certain embodiments, R¹¹ is an optionally substituted heteroaryl, such as a pyridyl group. An exemplary embodiment of R⁵ includes the structure:

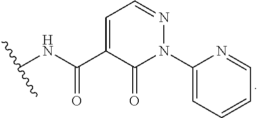

In certain embodiments of compounds of Formula Ia and Ib, R⁵ has the structure:

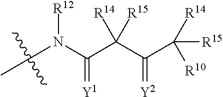

wherein R¹⁰, R¹², R¹⁴, R¹⁵, Y¹ and Y² are as defined herein. In certain embodiments, R¹⁴ and R¹⁵ together with the atoms to which they are attached form an optionally substituted carbocyclic ring.

A particular example of R⁵ is the structure:

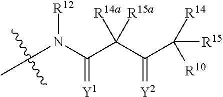

wherein R¹⁰, R¹², Y¹ and Y² Y are as defined herein and R^{14a} and R^{15a} together with the carbon atom to which they are both attached form a spirocyclic carbocycle, such as a cyclopropylidine group.

In certain embodiments, Y¹ is O.

In certain embodiments, Y² is O.

In certain embodiments, R¹⁴ and R¹⁵ are H.

In certain embodiments, R¹⁰ is phenyl optionally substituted with a halogen group. In certain embodiment, said phenyl is substituted with F.

For example, in certain embodiments R⁵ is selected from the structures:

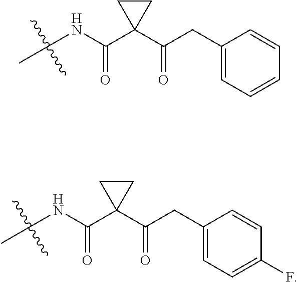

In certain embodiments of compounds of Formula Ia and Ib, R⁵ has the structure:

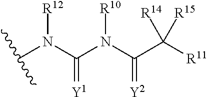

wherein Y¹, Y², R¹⁰, R¹¹, R¹², R¹⁴ and R¹⁵ are as defined herein. In certain embodiments, R¹¹ is optionally substituted aryl.

In certain embodiments, R¹² is H or C₁-C₆ alkyl.

In certain embodiments, R¹⁰ is H or C₁-C₆ alkyl.

In certain embodiments, R¹⁴ is H.

In certain embodiments, R¹⁵ is H.

In certain embodiments, R¹¹ is phenyl optionally substituted with halogen, for example a fluoro group.

For example, in certain embodiments R⁵ is:

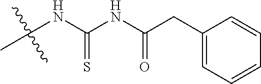

A further exemplary embodiment of R⁵ is:

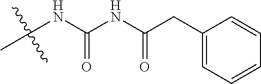

In certain embodiments of compounds of Formula Ia and Ib, R⁵ has the following structure:

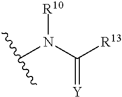

wherein Y, R¹⁰ and R¹³ are as defined herein.

In certain embodiments, Y is O.

In certain embodiments, R¹⁰ is H.

In certain embodiments, R¹⁰ is CH₂Ph.

In certain embodiments, R¹³ is alkyl, (CR¹⁴R¹⁵)ₙ—O—(CR¹⁴R¹⁵)ₘ-aryl, (CR¹⁴R¹⁵)-aryl, (CR¹⁴R¹⁵)-heteroaryl, (CR¹⁴R¹⁵)-heterocyclyl, (CR¹⁴R¹⁵)—N(SO₂R^{a})(CR¹⁴R¹⁵)R¹¹, or (CR¹⁴R¹⁵)NR¹⁰C(═O)-aryl, wherein said alkyl, aryl, heteroaryl and heterocyclyl portions are optionally substituted.

In particular embodiments, R¹³ is CR¹⁴R¹⁵O(CH₂)ₘ-phenyl, wherein phenyl is optionally substituted with halogen (for example Cl), R¹⁴ and R¹⁵ are independently H or methyl and m is 0 or 1.

In particular embodiments, R¹³ is OR^{a}, wherein R^{a} is C₁-C₆ alkyl or phenyl.

In particular embodiments, R¹³ is (C₁-C₃ alkyl)-phenyl.

In particular embodiments, R¹³ is (C₁-C₂ alkyl)-hetAr wherein hetAr is a 6 membered heteroaryl ring having one or two ring nitrogen atoms. A particular example of R¹³ is (C₁-C₂ alkyl)-pyridyl.

In particular embodiments, R¹³ is a 5-6 membered heteroaryl ring having 1 to 2 ring atoms independently selected from N, O and S and optionally substituted with one or two groups independently selected from NH-phenyl, morpholinyl, phenyl, and C₁-C₆ alkyl.

In particular embodiments, R¹³ is phenyl optionally substituted with one or two groups independently selected from CN, F, phenyl, O-phenyl, N(C₁-C₆ alkyl)₂, and NHC(═O)(C₁-C₆ alkyl).

In particular embodiments, R¹³ is CH₂—N(C₁-C₄ alkyl)SO₂R^{a} or CH₂—N(CH₂Ph)SO₂R^{a}. In particular embodiments, R^{a} is C₁-C₆ alkyl, phenyl or a 5 membered heteroaryl ring having one or two ring heteroatoms independently selected from N and O and optionally substituted with C₁-C₆ alkyl.

In certain embodiments, R¹³ is (CH₂)ₙ-hetCyc wherein n is 0 or 1 and hetCyc is a saturated or partially saturated 6 membered heterocyclic ring having a ring nitrogen atom and optionally substituted with oxo, C(═O)(C₁-C₆ alkyl), SO₂(C₁-C₆ alkyl), SO₂-phenyl or C(O)O(C₁-C₆ alkyl).

In particular embodiments, R¹³ is C₁-C₆ alkyl optionally substituted with (C₃-C₆)cycloalkyl or O—(C₁-C₆ alkyl).

In particular embodiments, R¹³ is CH₂N(C —C₆ alkyl)C(═O)phenyl.

For example, in certain embodiments R⁵ is selected from the structures:

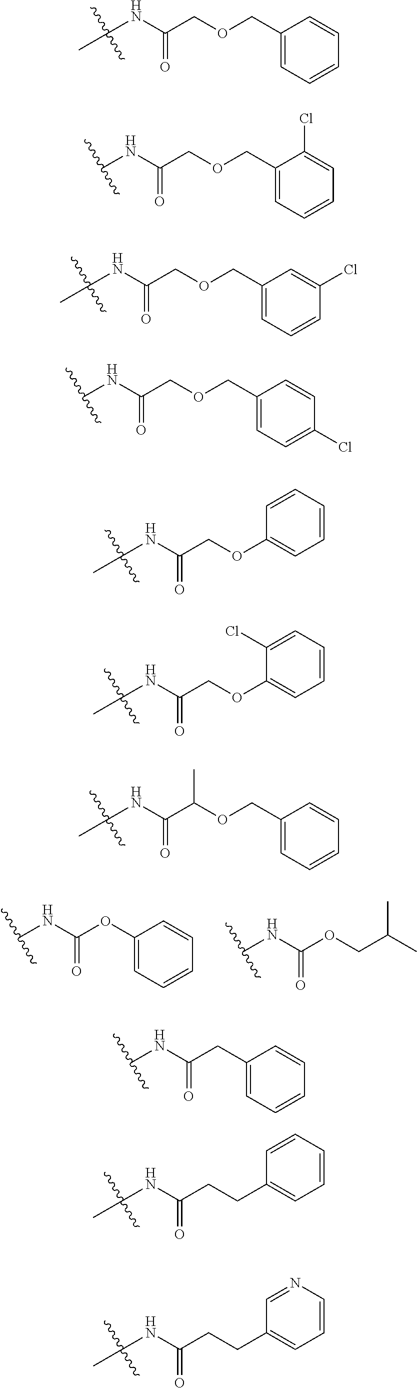

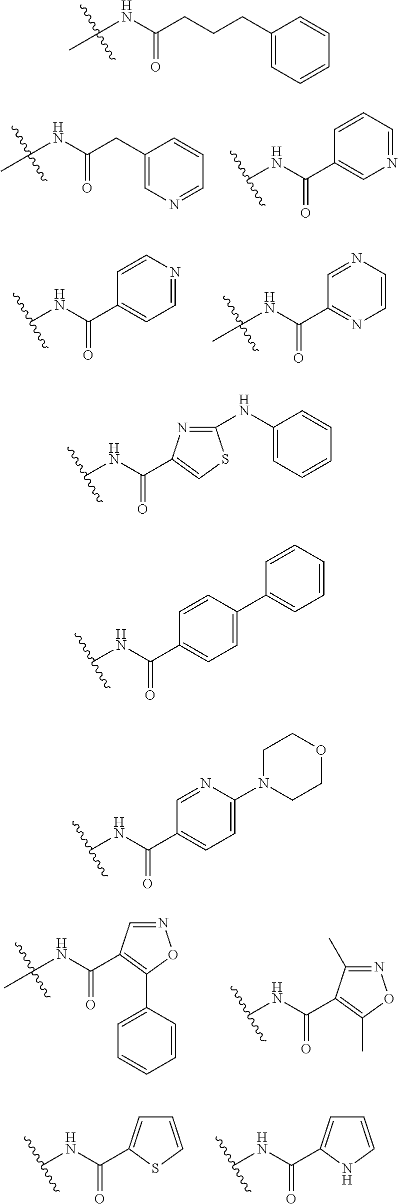

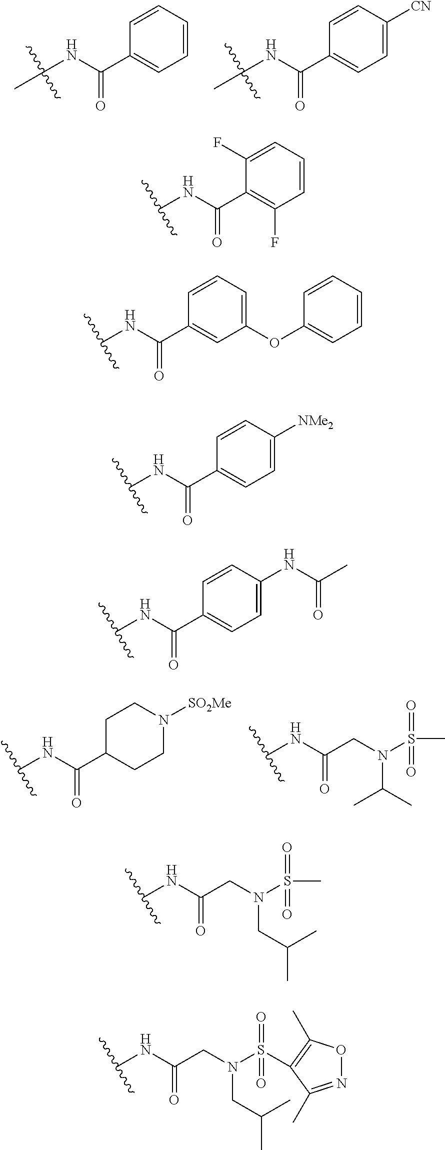

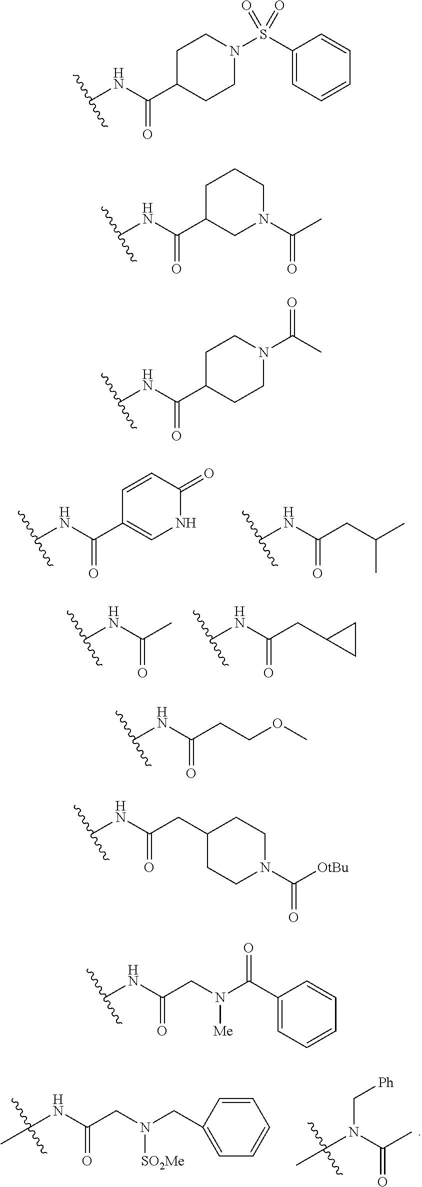

In certain embodiments of compounds of Formula Ia and Ib, R⁵ has the following structure:

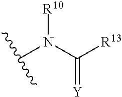

wherein Y and R¹⁰ are as defined herein and R¹³ is alkyl or (CR¹⁴R¹⁵)-hetAr. In certain embodiments, R¹⁴ and R¹⁵ are H. In other embodiments, R¹⁴ and R¹⁵ together with the carbon to which they are attached from a cyclopropylidine ring. In certain embodiments, Y is O. In certain embodiments, hetAr is a 5-9 membered monocyclic or bicyclic ring having one or two ring heteroatoms independently selected from N and O. Exemplary embodiments of R⁵ include the structures:

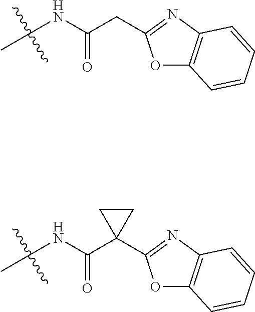

In certain embodiments of compounds of Formula Ia and Ib, R⁵ has the structure:

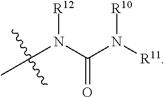

wherein R¹⁰, R¹¹, and R¹² are as defined herein.

In certain embodiments, R¹¹ is optionally substituted aryl or heteroaryl.

In certain embodiments, R¹¹ is a 5-10 membered monocyclic or bicyclic heteroaryl having a ring nitrogen atom and optionally having a second heteroatom selected from N and O, wherein said heteroaryl is optionally substituted with C₁-C₆ alkyl.

In certain embodiments, R¹² is H.

In certain embodiments, R^{io} is H or methyl.

For example, in certain embodiments R⁵ is selected from the structures:

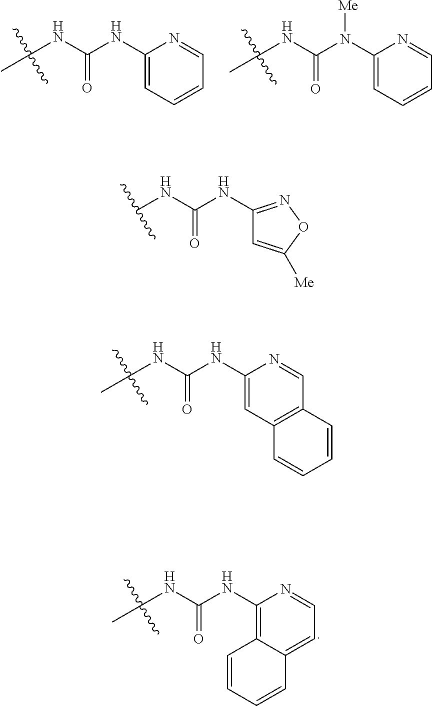

In other embodiments, R¹⁰ and R¹² together with the atoms to which they are attached form an oxo-substituted heterocyclic ring, wherein said heterocyclic ring is optionally fused to a phenyl ring. For example, in certain embodiments R⁵ is selected from the structures:

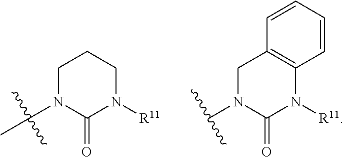

In a particular embodiment, R¹¹ is H.

In certain embodiments of compounds of Formula Ia and Ib, R⁵ is NR¹²SO₂R¹⁰, wherein R¹⁰ and R¹² are as defined herein.

In certain embodiments, R¹² is H.

In certain embodiments, R¹⁰ is phenyl optionally substituted with halogen, O—(C₁-C₆ alkyl), or C(═O)NH(C₁-C₆ alkyl).

In certain embodiments, R¹⁰ is an optionally substituted aryl. Exemplary embodiments of R⁵ include the structures:

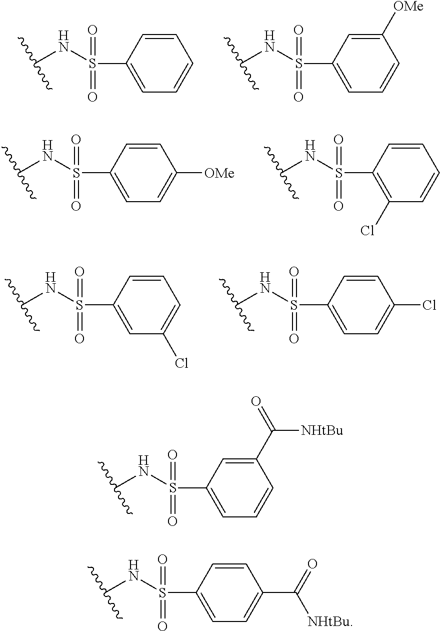

In certain embodiments of compounds of Formula Ia and Ib, R⁵ is NR¹²C(═O)C(═O)NR¹⁰R¹¹, wherein R¹⁰, R¹¹ and R¹² are as defined herein.

In certain embodiments, R¹¹ is H.

In certain embodiments, R¹² is H.

In certain embodiments, R¹⁰ is H, C₁-C₆ alkyl, (CH₂)₀₋₂-phenyl optionally substituted with halogen, or a 5 membered azacyclic ring such as pyrrolidinyl.

For example, in certain embodiments R⁵ is selected from the structures:

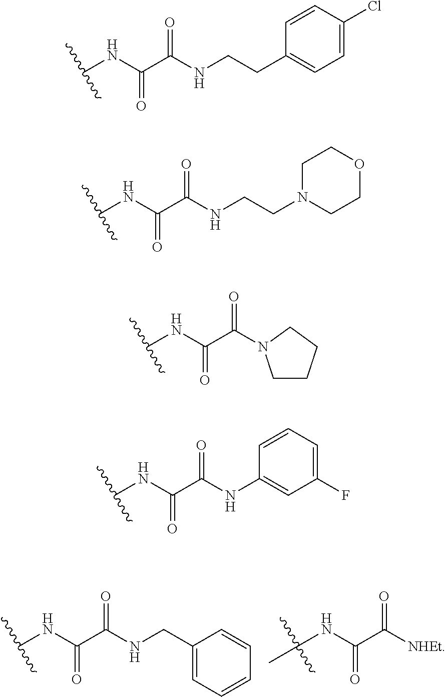

In certain embodiments of compounds of Formula Ia and Ib, R⁵ is NR¹²C(═O)C(═O)OR^{a}, wherein R¹² and R^{a} are as defined herein.

In certain embodiments, R¹² is H.

In certain embodiments, R^{a} is C₁-C₆ alkyl.

For example, in certain embodiments R⁵ is

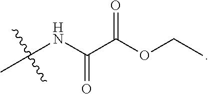

In certain embodiments of compounds of Formula Ia and Ib, R⁵ is an optionally substituted heteroaryl. For example, in certain embodiments, R⁵ is selected from the structures:

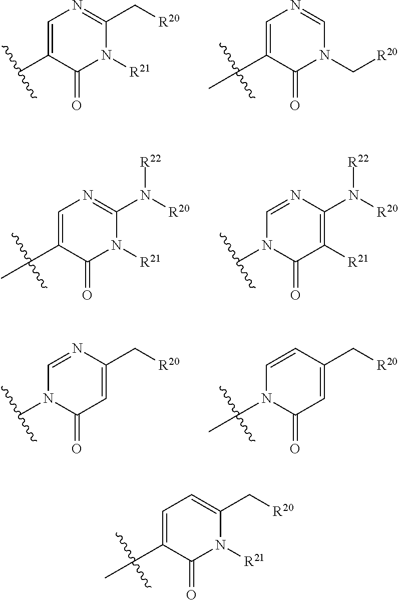

wherein R²⁰ is alkyl, cycloalkyl, aryl, or heteroaryl, and R²¹ and R²² are independently selected from H or alkyl, wherein said alkyl, cycloalkyl, aryl, and heteroaryl are optionally substituted with one or more groups independently selected from F, Cl, Br, I, alkyl and C₃-C₆ cycloalkyl.

Exemplary embodiments of R⁵ include the following structures:

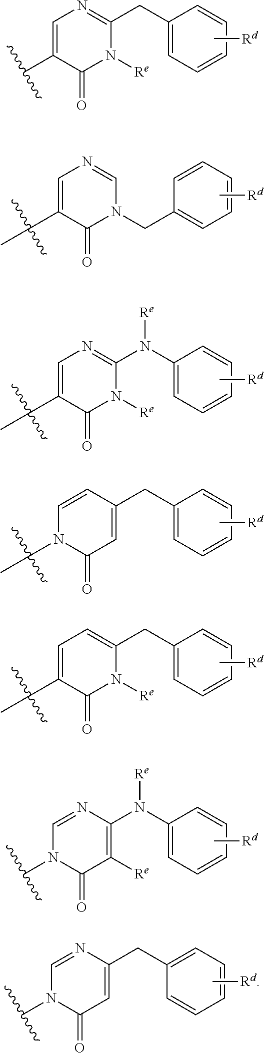

wherein R^{d} is as defined herein and R^{e} is H or an optionally substituted C₁-C₄ alkyl.

In certain embodiments, the phenyl group is substituted with one R^{d} group.

In certain embodiments, R^{d} is F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl.

In certain embodiments, R^{e} is independently H or C₁-C₄ alkyl.

Further exemplary embodiments of R⁵ include the structures:

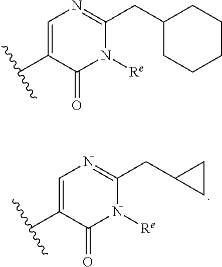

Particular embodiments of R⁵ include the structures:

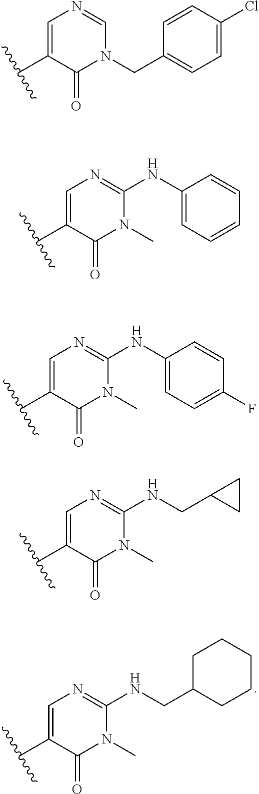

In certain embodiments of compounds of Formula Ia and Ib, R⁵ is NR¹⁰R¹¹. In certain embodiments, R¹⁰ is H. In certain embodiments, R¹¹ is hetAr, wherein hetAr is a substituted or unsubstituted 5-6 membered heteroaryl group having at least one ring nitrogen atom and optionally having a second ring heteroatom selected from N and O. Examples of hetAr include pyridyl, isoxazolyl, and pyridazinyl groups. In certain embodiments, hetAr is substituted with one or two groups independently selected from C₁-C₆ alkyl and C(═O)NR^{a}R^{b}. In certain embodiments, Ra is H. In certain embodiments, R^{b} is phenyl optionally substituted with a halogen group. In certain embodiment, R^{b} is C₁-C₆ alkyl, such as, but not limited to, methyl, ethyl or isopropyl. In certain embodiments, R^{b} is a 6 membered heteroaryl having at least one nitrogen atom, for example pyridyl.

Exemplary embodiment of R⁵ includes the structures:

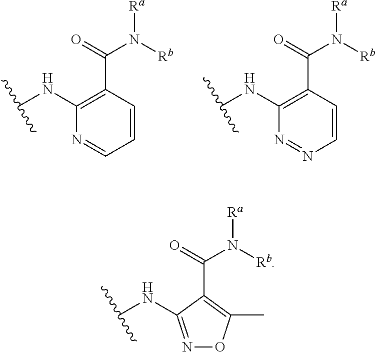

A particular embodiment of R⁵ is the structure:

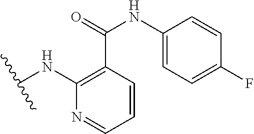

Particular embodiments of R³ include the structures:

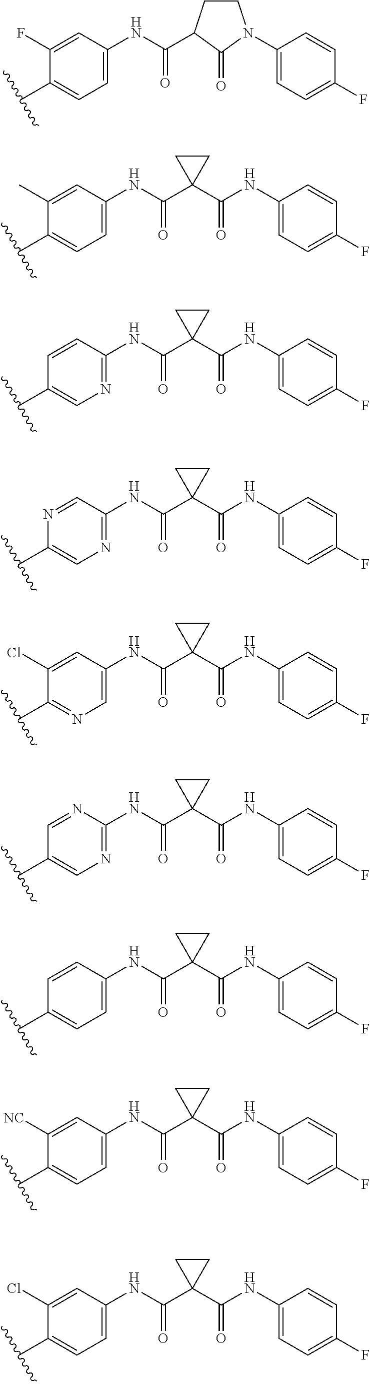

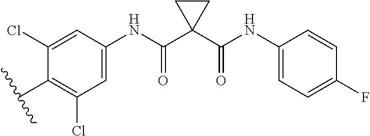

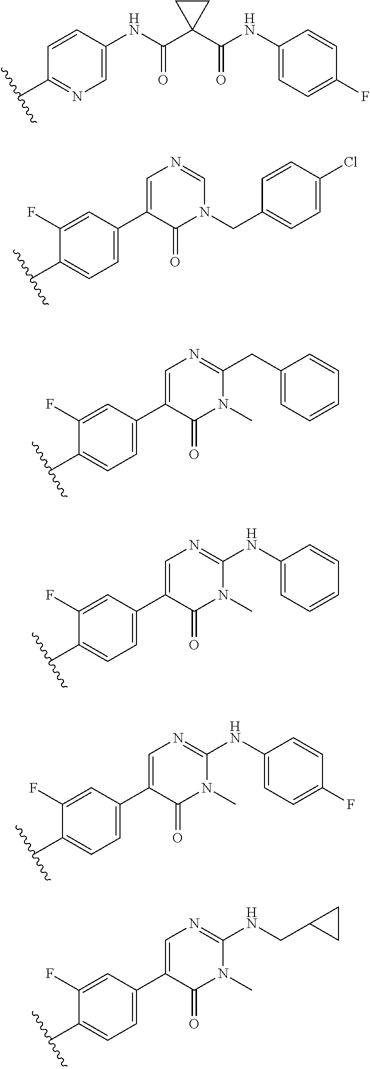

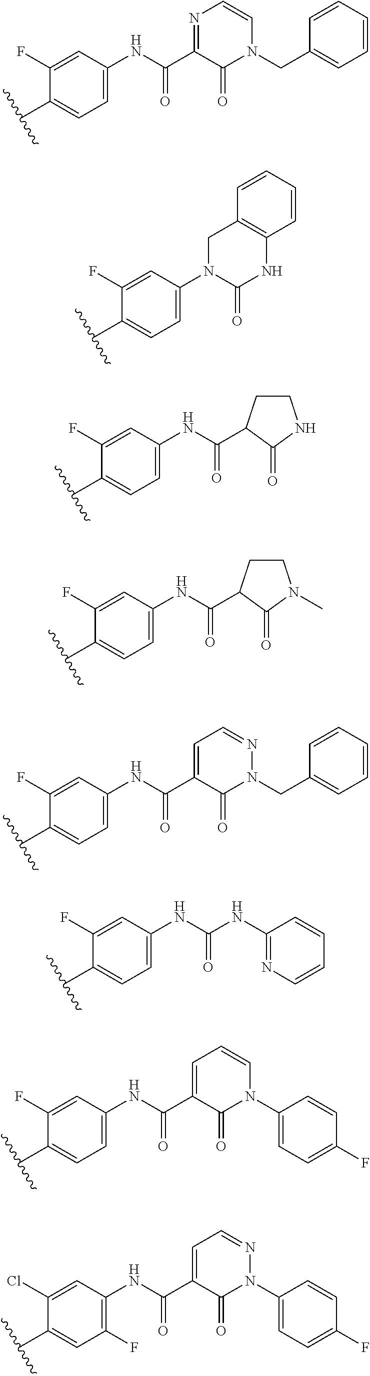

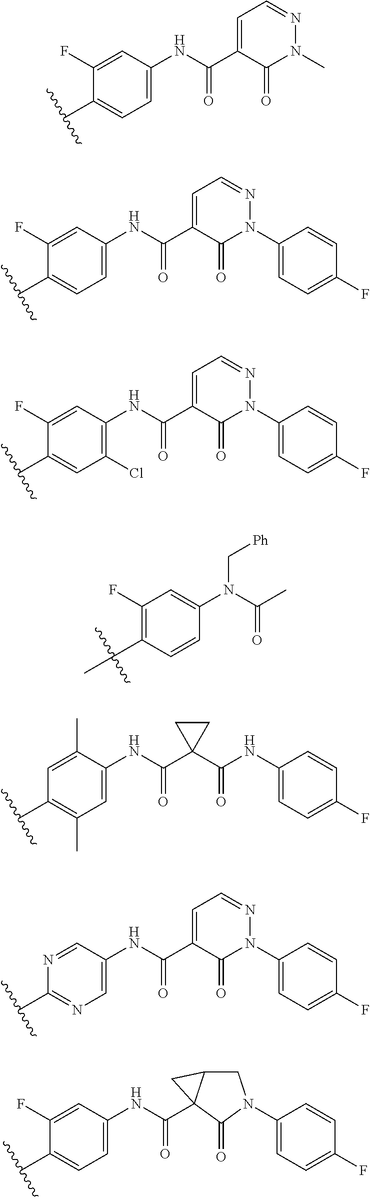

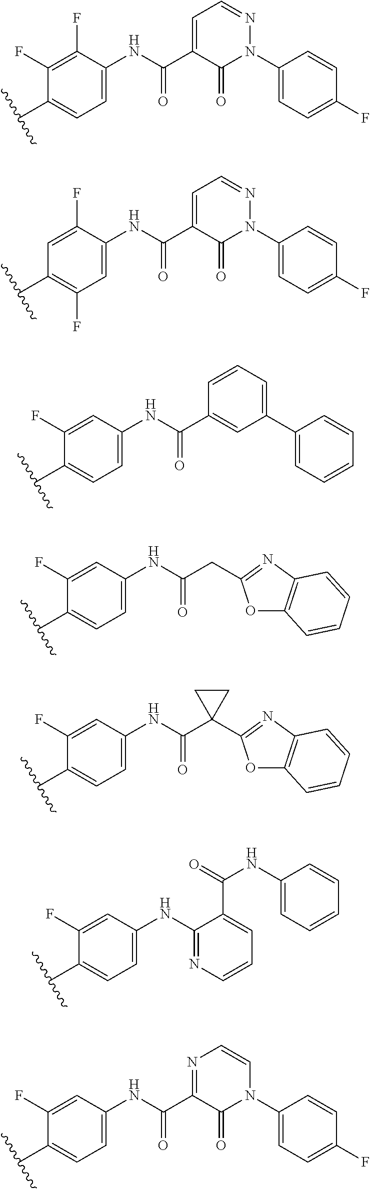

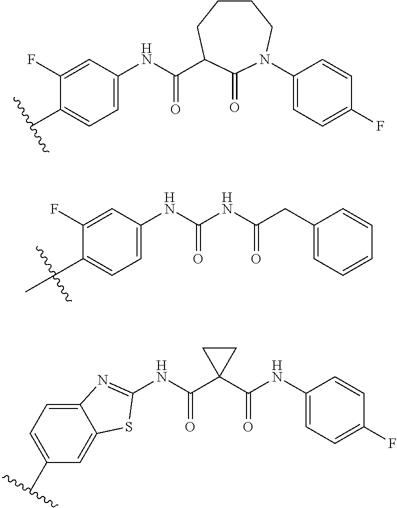

The heterobicyclic pyrazole compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In addition, the present invention embraces all geometric and positional isomers. For example, if a heterobicyclic pyrazole compound of the present invention incorporates a double bond or a fused ring, the cis- and trans-forms, as well as mixtures thereof, are embraced within the scope of the invention. Both the single positional isomers and mixture of positional isomers, e.g., resulting from the N-oxidation of the pyrimidine and pyrazine rings, are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The present invention also embraces isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and, their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶C1, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Synthesis of cMet Inhibitor Compounds

Heterobicyclic pyrazole compounds of Formula Ia and Ib of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

In certain embodiments, compounds of Formula Ia or Ib may be readily prepared using procedures well-known to prepare pyrazolo[3,4-b]pyridines (6531475, WO 01/098301, WO 01/081348, and WO 99/030710); and other heterocycles, which are described in: Comprehensive Heterocyclic Chemistry, Editors Katrizky and Rees, Pergamon Press, 1984; Klemm et al. (1970) J. Hetero. Chem. 7(2):373-379; Klemm et al. (1974) J. Hetero. Chem. 11(3): 355-361; Klemm et al. (1976) J. Hetero. Chem. 13:273-275; Klemm et al. (1985) J. Hetero. Chem. 22(5):1395-1396; Bisagni et al. (1974) Bull. Soc. Chim. Fr. (3-4, Pt. 2):515-518; Frehel et al. (1984) Heterocycles 22(5):1235-1247; WO 93/13664; WO 2004/012671; WO 2005/061476; U.S. Application Publication Nos. 2003/0045540, US 2003/0105089, and 2004/0024210; and U.S. Pat. Nos. 5,252,581, 6,232,320, and 6,579,882.

Compounds of Formula Ia and Ib may be prepared singly or as compound libraries comprising at least 2, for example 5 to 1,000 compounds, or 10 to 100 compounds. Libraries of compounds of Formula Ia or Ib may be prepared by a combinatorial ‘split and mix’ approach or by multiple parallel syntheses using either solution phase or solid phase chemistry, by procedures known to those skilled in the art. Thus according to a further aspect of the invention there is provided a compound library comprising at least 2 compounds, or pharmaceutically acceptable salts thereof.

For illustrative purposes, Schemes 1-15 show general methods for preparing the compounds of the present invention as well as key intermediates. For a more detailed description of the individual reaction steps, see the examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In preparing compounds of Formula I, protection of remote functionality (e.g., primary or secondary amine) of intermediates may be necessary. The need for such protection will vary depending on the nature of the remote functionality and the conditions of the preparation methods. Suitable amino-protecting groups (NIH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

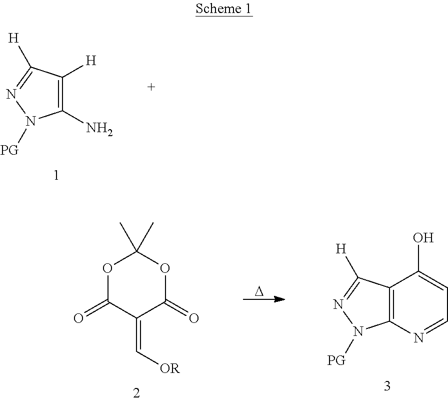

Scheme 1 shows a general scheme for the synthesis of intermediate compound 3, which is useful for the synthesis of compounds of Formula I. As shown in Scheme 1, reaction of a 5-aminopyrazole 1 (see, Misra, R. N., et al., Bioorg. Med. Chem. Lett. 2003, 13, 1133-1136), wherein N1 is protected by an appropriate protecting group (PG may be p-methoxybenzyl, phenylsulfonyl, or the like), with a vinyl ether of Meldrum's acid 2 (R=alkyl, such as methyl or ethyl) upon heating provides a Meldrum's acid enamine of the 5-aminopyrazole (not shown). Such an enamine can be cyclized upon heating to provide phenol 3.

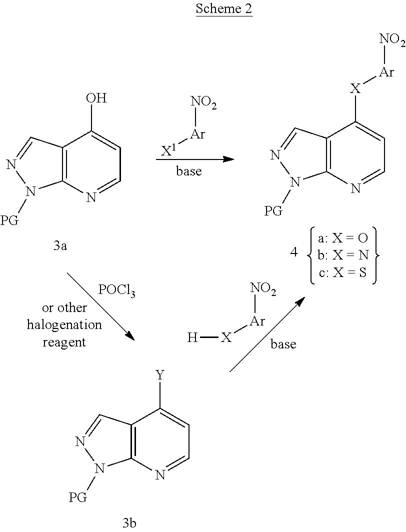

Scheme 2 shows methods for preparing intermediates 4 a-c. Intermediate 4a can be prepared by nucleophilic substitution of phenol 3a with a compound of the formula X¹—Ar—NO₂ (wherein X¹ is F, Cl, triflate or other appropriate leaving group and Ar is an aryl or heteroaryl ring as defined herein) in the presence of an appropriate base (e.g. Cs₂CO₃, NaH, KOt-Bu, DMAP, or the like). Conversion of the phenol 3a to an aryl halide 3b (Y=halogen or other leaving group such as triflate, etc.) can be achieved upon reaction with an appropriate electrophilic reagent (e.g. POCl₃, oxalyl chloride, NCS/PPh₃, POBr₃, NBS/PPh₃, CF₃SO₂Cl/2,6-lutidine, etc.). Nucleophilic substitution of aryl halide 3b with a compound of the formula HX—Ar—NO₂, wherein X is O, N or S, and Ar is an aryl or heteroaryl ring as definaherein, can be conducted using an appropriate base (e.g. Cs₂CO₃, NaH, KOt-Bu, DMAP, or the like) to give intermediates 4 a-c.

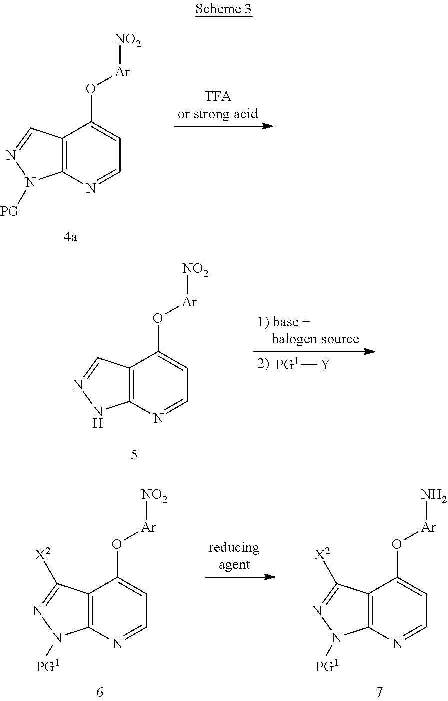

Scheme 3 shows a general scheme for the synthesis of intermediate 7, which are useful for the synthesis of compounds of Formula I. As shown in Scheme 3, removal of protecting group PG from compound 4a (PG=p-methoxybenzyl, Boc, phenylsulfonyl, or other appropriate protecting group) using TFA, strong acid, or other deprotection conditions appropriate for PG removal provides intermediate 5. Substitution at the 3-position of the pyrazolopyridine core may be achieved by halogenation (using I₂, Br₂, NIS, NBS or other halogenation reagent) of intermediate 5 which may require the presence of a base such as KOH, KOt-Bu, n-BuLi or the like. An appropriated protecting group may then be introduced (PG¹=p-methoxybenzyl, Boc, phenylsulfonyl, or other appropriate protecting group) to give 6. Intermediate 6 may subsequently be reduced to give aniline 7 using an appropriate reducing agent (e.g. Zn, Fe, H₂/Pd, SnCl₂-2H₂O, or the like).

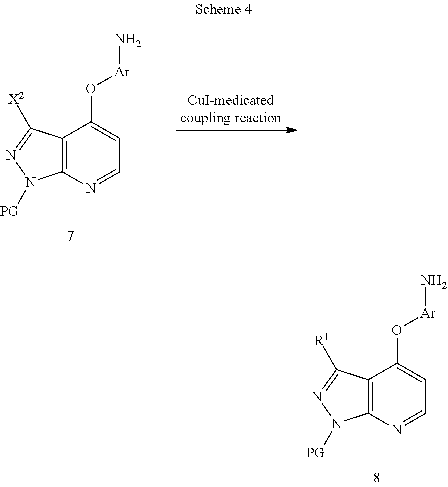

Scheme 4 shows a general scheme for the synthesis of intermediate 8, which is useful for the synthesis of compounds of Formula I. Intermediate 7 (X²=bromo or iodo) may be further elaborated at the 3-position by a CuI-mediated coupling reaction (or similar transition metal mediated coupling reactions known to those skilled in the art) to give intermediate 8, wherein R¹ is R¹W and where W is defined herein.

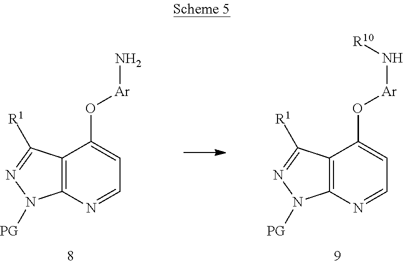

Scheme 5 shows a general scheme for the synthesis of amides, sulfonamides, carbamates, and ureas 9. Compounds 9 can be prepared by reaction of an amino-containing intermediate 8 with an activated carboxyl- or sulfonyl-containing reagent in the presence of an appropriate base (e.g. TEA, DIEA, N-methylmorpholine, pyridine, DMAP, or the like), as needed. Suitable carboxyl- or sulfonyl-containing reagents include, but are not limited to, acid chlorides, acid fluorides, sulfonyl chlorides, sulfonyl fluorides, polystyrene-2,3,5,6-tetrafluoro-4 -(methylcarbamoyl)phenol (PS-TFP)-carboxylates, PS-TFP-sulfonates, carbamoyl chlorides, isocyanates, isothiocyanates, anhydrides, chloroformates, HOBt ester, carbodiimide-derived O-acylurea, and the like. For example, compounds 9 wherein R¹⁰ is acyl, thiocarbonyl, carbamoyl, alkoxycarbonyl, or sulfonyl have been prepared by this method. Alternatively, intermediate 8 may be converted to compound 9 wherein R¹⁰ is alkyl by reductive alkylation methods. Intermediate 8 can also be coupled with an aryl or heteroaryl halide according to the procedures of Buchwald and Hartwig to provide a substituted amine 9 wherein R¹⁰=aryl or heteroaryl.

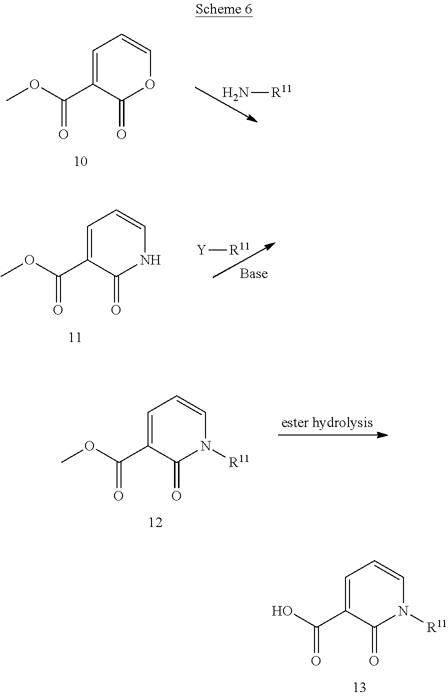

Scheme 6 shows routes for the preparation of acid intermediate 13. Acids of this type may be prepared from either reaction of the commercially available carboxypyrone ester 10 with an appropriate amine NH₂R¹¹ (wherein R¹¹ is, for example, alkyl, cycloalkyl, heterocyclyl, aryl or heteroaryl), or from the commercially available carboxy pyridone ester 11 via reaction with the appropriate activated electrophile Y—R¹¹ (wherein Y is an appropriate leaving group such as halogen, mesylate or tosylate; and R¹¹ is, for example, alkyl, cycloalkyl, or heterocyclyl) followed by hydrolysis of the resulting methyl ester 12 to the acid 13. The acid 13 may then be coupled to an appropriate aniline intermediate as in Schemes 5.

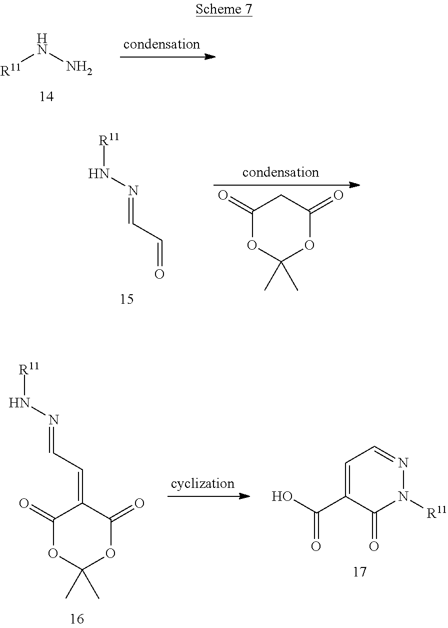

Scheme 7 shows a route for the preparation of acid intermediate 17 according to the general methods described by McNab H., et al., J. Chem. Soc. Perkin Trans. 1, 1982, 1845. Substituted hydrazine 14 (wherein R¹¹ is, for example, alkyl, cycloalkyl, heterocyclyl, aryl or heteroaryl) can be converted to hydrazono acetaldehyde 15 with standard dehydrating conditions such as in the presence of acetic acid at room temperature. The aldehyde/Meldrum's acid condensation product 16 is prepared in a suitable organic solvent such as toluene, benzene or dioxane at room temperature using piperidinium acetate as catalyst. Carboxylic acid pyridazinone 17 is prepared from hydrazono ethylidene 16 by cyclization under basic conditions (sodium methoxide in methanol) at 70° C. The acid can then be coupled to appropriate aniline intermediates as in Schemes 5.

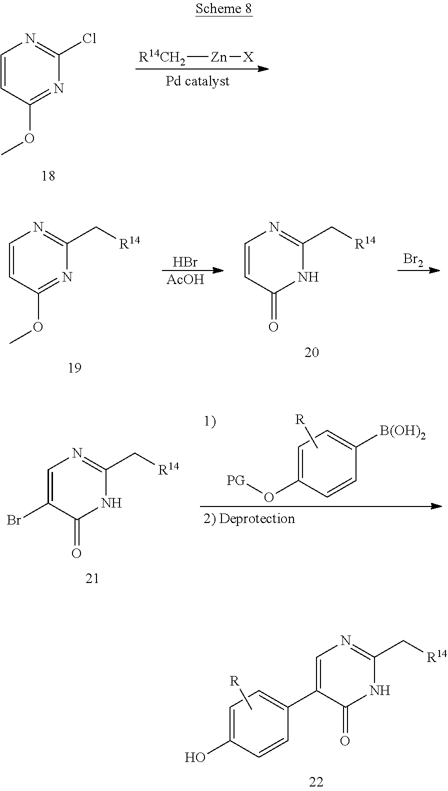

Scheme 8 shows a route for the preparation of phenol intermediate 22. Commercially available 2-chloro-4-methoxyppimidine 18 is reacted with the appropriate zinc reagent (wherein R¹⁴ is, for example, alkyl, cycloalkyl, heterocyclyl, heteroaryl or aryl) and palladium catalyst to give 2-substituted 4-methoxypyrimidine 19. Deprotection of the methoxypyrimidine with HBr in acetic acid provides 2-substituted pyrimidinone 20. Bromination in the 5-position gives pyrimidinone intermediate 21. Suzuki coupling of 21 to an appropriate boronic acid gives a bicyclic intermediate which after final deprotection of the phenol gives intermediate 22. Intermediate 22 can be substituted for a phenoxy nitro derivative and reacted with appropriate core intermediates as in Scheme 2.

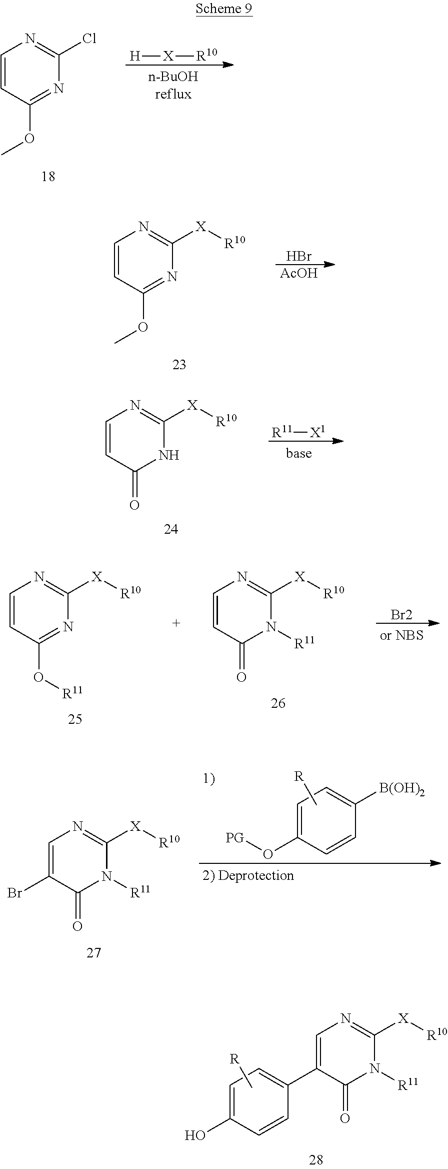

Scheme 9 shows a method for preparing phenol intermediate 28 (wherein R¹⁰ and R¹¹ are independently selected from H, alkyl, cycloalkyl, heterocyclic, aryl and heteroaryl). Nucleophilic substitution of 2-chloro-4-methoxypyrimidine 18 with a compound of the formula H—X—R¹⁰(wherein X is O, N or S) can be accomplished in an appropriate solvent such as n-butanol, at refluxing temperature. Deprotection of the methoxypyrimidine with HBr in acetic acid provides 2-substituted pyrimidinone 24. Alkylation of 24 to provide the 1-substituted pyrimidinone 26 can be accomplished with an alkylation agent R¹¹—X¹ (wherein X¹ is an appropriate leaving group such as halogen, mesylate, or tosylate) mediated by an appropriate base (e.g. sodium alkoxide, lithium or sodium hydride, or the like) providing a mixture of isomers 25 and 26. Isomers 25 and 26 can be separated using purification techniques known to those skilled in the art (e.g. flash chromatography, reverse phase HPLC, or the like). Bromination in the 5-position with a brominating agent such as Br₂ or NBS gives pyrimidinone intermediate 27. Suzuki coupling of 27 to an appropriate boronic acid gives a bicyclic intermediate which after final deprotection of the phenol gives intermediate 28 as described for Scheme 8. Intermediate 28 can be substituted for a phenoxy nitro derivative and reacted with appropriate core intermediates as in Scheme 2.

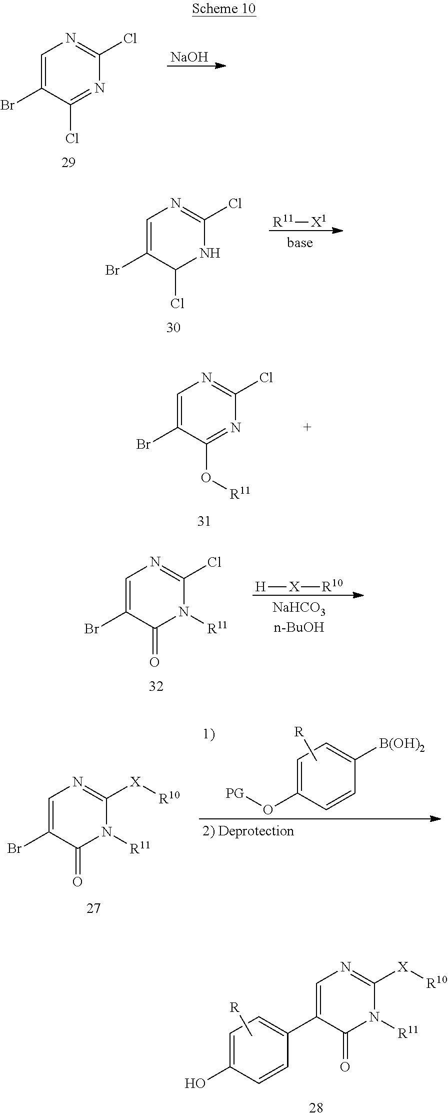

Alternatively, phenol intermediate 28 (wherein R¹⁰ and R¹¹ are independently selected from H, alkyl, cycloalkyl, heterocyclic, aryl and heteroaryl) can be prepared as shown in Scheme 10. 5-Bromo-2,4-dichloropyrimidine 29 is hydrolyzed with NaOH to give 5-bromo-2-chloropyrimidin-4(3H)-one 30 as described in EP 1506967A1. Alkylation of 30 to provide the 1-suubstituted pyrimidinone 32 can be accomplished with an alkylation agent R¹¹—X¹ (wherein X¹ is an appropriate leaving group such as halogen, mesylate, or tosylate) mediated by an appropriate base (e.g. sodium alkoxide, lithium or sodium hydride, or the like) providing a mixture of isomers 31 and 32. Isomers 31 and 32 can be separated using purification techniques known to those skilled in the art (e.g. flash chromatography, reverse phase HPLC, or the like). Nucleophilic substitution of 32 with a compound of the formula H—X—R¹⁰, wherein X is O, N or S) can be accomplished at elevated temperature with a base such as NaHCO₃ in an appropriate solvent such as n-butanol. Suzuki coupling of 27 to an appropriate boronic acid gives a bicyclic intermediate which after final deprotection of the phenol gives intermediate 28. Intermediate 28 can be substituted for a phenoxy nitro derivative and reacted with appropriate core intermediates as in Scheme 2.

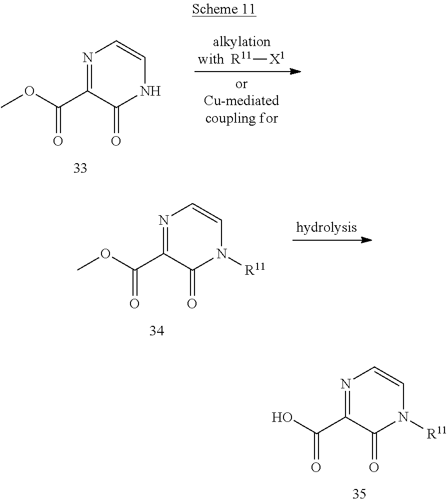

The substituted pyrazino carboxylic acid 35 can be prepared according to Scheme 11. Methyl 3-oxo-3,4-dihydropyrazine-2-carboxylate 33 can be converted to alkyl pyrazino carboxylate 34 by standard basic alkylation conditions using an appropriate alkyl halide R¹¹—X¹ (wherein R¹⁷ may be alkyl, cycloalkyl, or heterocyclic, and X¹ is an appropriate leaving group such as halogen, mesylate, or tosylate). Suitable alkylation conditions include but are not limited to K₂CO₃ in a suitable solvent such as acetone or DMF at room temperature or elevated temperature, or NaH in THF at ambient or elevated temperature followed by addition of R¹¹—X¹. In one embodiment, this alkylation can be achieved with Lill in DMF at 0° C., followed by addition of alkyl chloride or alkyl bromide or alkyl iodide and warming to room temperature. When R¹¹=aryl or heteroaryl, the pyrazinone ester 34 can be prepared by a copper mediated cross-coupling reaction with iodobenzene, CuI catalyst, a diamine ligand and an appropriate base in a suitable organic solvent such as THF, DMF, PhMe, MeCN or dioxane at elevated temperature. For example, in certain embodiments the reaction conditions include, Cul, N,N′-dimethylethylenediamine and K₃PO₄ in dioxane at 110° C. Carboxylic acid 35 can then be obtained using standard saponification conditions such as LiOH or NaOH in mixed aqueous/organic solvent systems. The acid 35 can then be coupled to appropriate aniline intermediates as in Scheme 5.

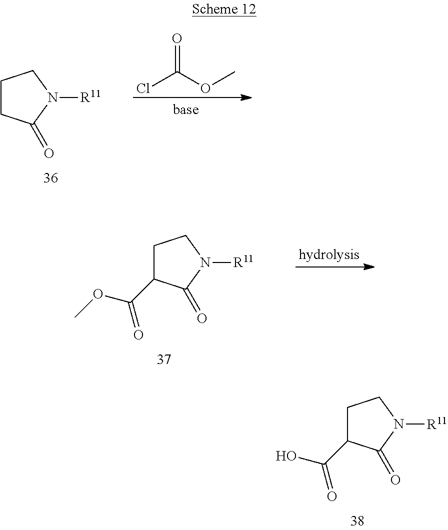

N-alkylated-2-oxopyrrolidine-3-carboxylic acid 38 (wherein R¹¹ may be alkyl, cycloalkyl, heterocyclic, heteroaryl, or aryl) may be synthesized according to Scheme 12. Compound 36 can be converted to ester 37 by reaction with methyl carbonochloridate or methyl carbono-brominate in the presence of an appropriate base (e.g. LDA, LHMDS, or the like). Carboxylic acid 38 can then be prepared from 37 by ester hydrolysis as described for Scheme 11 or using potassium trimethylsilanolate, or the like. The acid 38 can then be coupled to appropriate aniline intermediates as in Schemes 5.

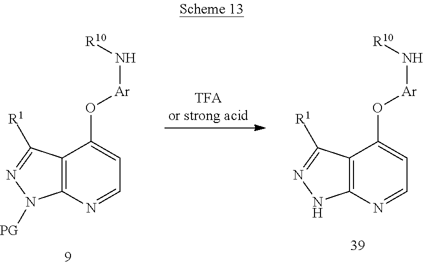

Scheme 13 shows a method for the preparation of compound 39, wherein Ar is an aryl or heteroaryl ring as defined herein, R¹ is alkoxy and thio, and R¹⁰ is as described for Scheme 5. Compound 39 can be prepared from compound 9 (prepared as in Scheme 5) by removal of protecting group PG (e.g. p-methoxybenzyl, phenylsulfonyl, or the like) by heating (40-80° C.) as needed with TFA or strong acid, or using alternative deprotection conditions as necessary to remove PG (see T. W. Greene and P. G. M. Wuts, Protecting Groups in Organic Synthesis, Second Edition, Wiley, New York, 1991).

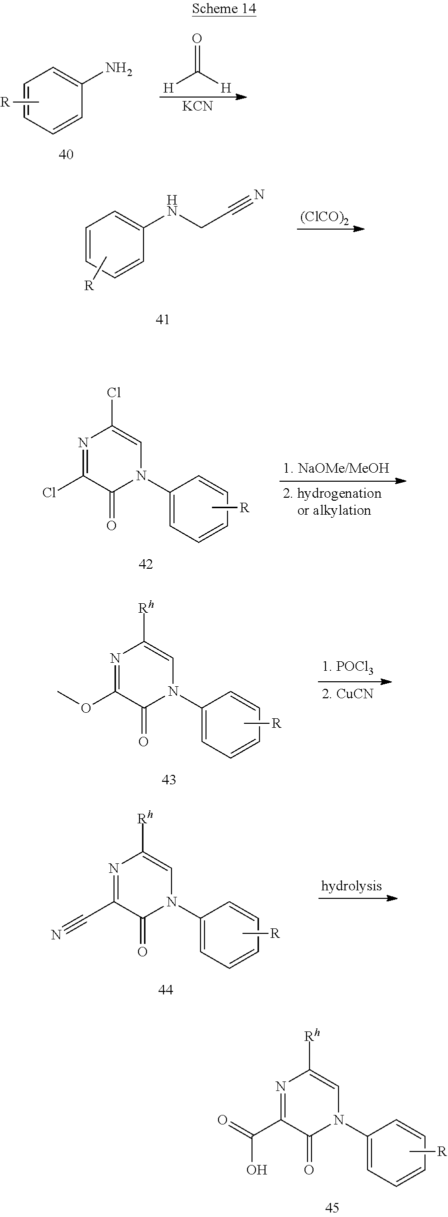

Scheme 14 shows a route for the preparation of pyrazinone acid intermediate 45 wherein R^{h} is independently selected from H, alkyl, cycloalkyl, heterocyclic, or heteroaryl, which is useful for the synthesis of compounds of Formula I. Substituted aniline 40 can be converted to amino acetonitrile compound 41 by treating with KCN and a formaldehyde equivalent with standard dehydrating conditions such as in the presence of acetic acid at room temperature. The cyclization product 42 is prepared by treating 41 with oxalyl dichloride in a suitable organic solvent such as dichlorobenzene at elevated temperature (about 100° C.). Pyrazinone 43 can be made in a two step sequence from the 3,5-dichloro pyrazinone compound 42. First, compound 42 is treated with sodium methoxide in a suitable organic solvent such as MeOH or THF or MeOH/THF mixture at temperatures ranging from about 0° C. to reflux, followed by conversion of the intermediate 5-chloropyrazinone (not shown) to the 5-H pyrazinone 43. The conversion can be carried out either under reductive conditions, or, when R^{h} is alkyl, cycloalkyl, heterocyclic, or heteroaryl, using Pd mediated cross-coupling conditions. Nitrile 44 can be synthesized from methoxy pyrazinone 43 by chlorination followed by nitrilation. The chlorination can be accomplished with POCl₃, thionyl chloride, oxalyl chloride, or PCl₅. Preferably, this transformation is achieved with POCl₃ using DMF as solvent at elevated temperature (about 90° C.). Nitrilation can be achieved by standard conditions with CuCN in a suitable organic solvent such as NMP at elevated temperature (about 150° C.). Carboxylic acid pyrazinone 45 can be made in a three step, one-pot reaction. First, nitrile compound 44 is treated with concentrated H₂SO₄ neat at room temperature. The resulting amide intermediate is then treated with MeOH, and this mixture is refluxed to generate methyl ester pyrazinone intermediate. Then desired carboxylic acid pyrazinone 45 can be prepared by basic bydrolysis of the methyl ester pyrazinone intermediate under standard conditions using either NaOH or LiOH in standard mixture aqueous/organic solvent systems. The acid 45 may then be coupled to an appropriate aniline intermediate as in Schemes 5 to provide compounds of Formula I.

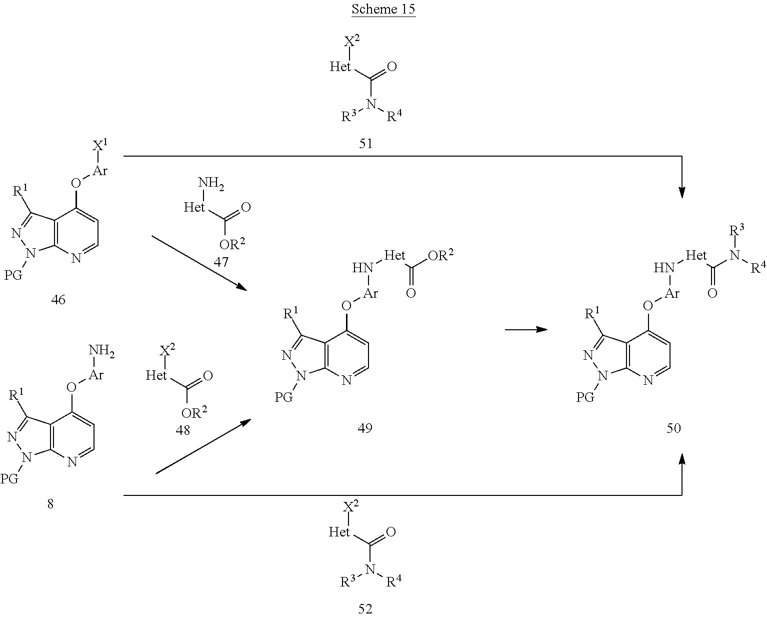

Scheme 15 shows a general scheme for the synthesis of intermediate 50, wherein Het is a substituted or unsubstituted 5-6 membered heteroaryl group having at least one ring nitrogen atom and optionally having a second ring heteroatom selected from N and O. Intermediate compounds 50 are useful for the synthesis of compounds of Formula I. As shown in Scheme 15, elaboration of the pyrazolopyridine 4-position phenoxy group into an amino linked heteroaryl amide may proceed via several pathways. Intermediate 46 bearing an appropriate leaving group X¹ may be reacted with a heteroaryl amino ester 47 typically under transition metal catalysis to provide ester 49. Ester 49 may then be converted to compound 50 using standard ester hydrolysis conditions followed by standard amide bond forming conditions. Alternatively, 46 may be reacted with a heteroaryl amino amide 51 under transition metal catalysis to give intermediate 50 directly. Alternatively, the mode of coupling may be reversed, wherein an intermediate 8 bearing an amino group may be reacted with a heteroaryl ester 48 bearing leaving group X² typically under transition metal catalyzed or thermal conditions to give intermediate 49. Intermediate 49 may then be converted to intermediate 50 using standard ester hydrolysis conditions followed by standard amide bond forming conditions. Alternatively, 8 may be reacted with a heteroaryl amide 52 bearing leaving group X², typically under transition metal catalyzed or thermal conditions to give intermediate 50 directly. When R¹ is an appropriate substituent, intermediate 50 may be deprotected to give final compounds of Formula I.

### Methods of Separation

In the methods of preparing the compounds of this invention, it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, a-methyl-13- phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem., (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

### Biological Evaluation

Determination of the activity of c-Met kinase activity of a compound of Formula Ia or Ib is possible by a number of direct and indirect detection methods. One example of an assay used for the determination of c-Met kinase activity is based on an enzyme linked immunosorbant assay (ELISA). The assay includes a compound of Formula Ia or Ib, c-Met (His-tagged recombinant human Met (amino acids 974-end), expressed by baculovirus), and ATP in assay buffer, as described in Example A.

In MKN45 cells, the activity of cMet inhibitors of Formulas Ia and Ib was determined by the in vitro fluorescence assay as described in Example B.

Certain exemplary compounds described herein were prepared, characterized, and assayed for their c-Met binding activity and in vitro activity against tumor cells. The range of c-Met binding activities was less than 1 nM to about 10 μM. Certain exemplary compounds of the invention had c-Met binding activity IC₅₀ values less than 10 nM. Certain compounds of the invention had MKN45 cell-based activity IC₅₀ values less than 100 nM.

### Administration of Compounds of Formulas Ia and Ib

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

Methods of Treatment with Compounds of Formulas Ia or Ib

Compounds of the present invention are useful for treating diseases, conditions and/or disorders including, but not limited to, those characterized by over expression of receptor tyrosine kinases (RTK), e.g. c-Met kinase. Accordingly, another aspect of this invention includes methods of treating or preventing diseases or conditions that can be treated or prevented by inhibiting receptor tyrosine kinases (RTK), including c-Met. In one embodiment, the method comprises administering to a mammal in need thereof a therapeutically effective amount of a compound of Formula Ia or Ib, or a stereoisomer, geometric isomer, tautomer, solvate, metabolite, or pharmaceutically acceptable salt or prodrug thereof.

Diseases and conditions treatable according to the methods of this invention include, but are not limited to, cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient. In one embodiment, a human patient is treated with a compound of Formula Ia or Ib and a pharmaceutically acceptable carrier, adjuvant, or vehicle, wherein said compound of Formula Ia or Ib is present in an amount to detectably inhibit cMet kinase activity.

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

Cardiovascular diseases which can be treated according to the methods of this invention include, but are not limited to, restenosis, cardiomegaly, atherosclerosis, myocardial infarction, and congestive heart failure.

Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Inflammatory diseases which can be treated according to the methods of this invention include, but are not limited to, rheumatoid arthritis, psoriasis, contact dermatitis, and delayed hypersensitivity reactions.

Another aspect of this invention provides a compound of this invention for use in the treatment of the diseases or conditions described herein in a mammal, for example, a human, suffering from such disease or condition. Also provided is the use of a compound of this invention in the preparation of a medicament for the treatment of the diseases and conditions described herein in a warm-blooded animal, such as a mammal, for example a human, suffering from such disorder.

### Pharmaceutical Formulations

In order to use a compound of this invention for the therapeutic treatment (including prophylactic treatment) of mammals including humans, it is normally formulated in accordance with standard pharmaceutical practice as a pharmaceutical composition. According to this aspect of the invention there is provided a pharmaceutical composition comprising a compound of this invention in association with a pharmaceutically acceptable diluent or carrier.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of Formula Ia or Ib having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compound of this invention for use herein is preferably sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution.

The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds of Formulas I may be prepared. Suitable examples of sustained-release preparations include semipermeable matrices of solid hydrophobic polymers containing a compound of Formula Ia or Ib, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of Formula Ia or Ib suitable for oral administration may be prepared as discrete units such as pills, capsules, cachets or tablets each containing a predetermined amount of a compound of Formula Ia or Ib.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formula Ia or Ib intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of Formula Ia or Ib compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formula Ia or Ib may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

Also provided are compositions comprising a compound of claim 1 in an amount to detectably inhibit Met kinase activity and a pharmaceutically acceptable carrier, adjuvant, or vehicle.

### Combination Therapy

The compounds of Formulas Ia and Ib may be employed alone or in combination with other therapeutic agents for the treatment of a disease or disorder described herein, such as a hyperproliferative disorder (e.g., cancer). In certain embodiments, a compound of Formula Ia or Ib is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of Formula Ia or Ib such that they do not adversely affect each other. Such compounds are suitably present in combination in amounts that are effective for the purpose intended. In one embodiment, a composition of this invention comprises a compound of Formula Ia or Ib, or a stereoisomer, geometric isomer, tautomer, solvate, metabolite, or pharmaceutically acceptable salt or prodrug thereof, in combination with a chemotherapeutic agent such as described herein.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In a particular embodiment of anti-cancer therapy, a compound of Formula Ia or Ib, or a stereoisomer, geometric isomer, tautomer, solvate, metabolite, or pharmaceutically acceptable salt or prodrug thereof, may be combined with other chemotherapeutic, hormonal or antibody agents such as those described herein, as well as combined with surgical therapy and radiotherapy. Combination therapies according to the present invention thus comprise the administration of at least one compound of Formula Ia or Ib, or a stereoisomer, geometric isomer, tautomer, solvate, metabolite, or pharmaceutically acceptable salt or prodrug thereof, and the use of at least one other cancer treatment method. The amounts of the compound(s) of Formula Ia or Ib and the other pharmaceutically active chemotherapeutic agent(s) and the relative timings of administration will be selected in order to achieve the desired combined therapeutic effect.

Also provided are compositions comprising a compound of Formula Ia or Ib in combination with an additional therapeutic agent selected from an anti-proliferative agent, an anti-inflammatory agent, an immunomodulatory agent, a neurotropic factor, an agent for treating cardiovascular disease, an agent for treating liver disease, an anti-viral agent, an agent for treating blood disorders, an agent for treating diabetes, or an agent for treating immunodeficiency disorders.

### Matabolites of Compounds of Formulas Ia and Ib

Also falling within the scope of this invention are the in vivo metabolic products of heterobicyclic pyrazole compounds of Formulas Ia and Ib described herein. Such products may result for example from the oxidation, reduction, hydrolysis, amidation, deamidation, esterification, deesterification, enzymatic cleavage, and the like, of the administered compound. Accordingly, the invention includes metabolites of compounds of Formulas Ia and Ib, including compounds produced by a process comprising contacting a compound of this invention with a mammal for a period of time sufficient to yield a metabolic product thereof.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

### Prodrugs of Compounds of Formulas Ia and Ib

In addition to compounds of Formulas Ia and Ib, the invention also includes pharmaceutically acceptable prodrugs of such compounds. Prodrugs include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, cirtulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of Formula Ia or Ib can be derivatized as an amide or alkyl ester. As another example, compounds of this invention comprising free hydroxy groups may be derivatized as prodrugs by converting the hydroxy group into a group such as, but not limited to, a phosphate ester, hemisuccinate, dimethylaminoacetate, or phosphoryloxymethyloxycarbonyl group, as outlined in Advanced Drug Delivery Reviews, (1996) 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group may be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Free amine groups of compounds of Formulas Ia and Ib can also be derivatized as amides, sulfonamides or phosphonamides. All of these moieties may incorporate groups including, but not limited to, ether, amine and carboxylic acid functionalities. For example, a prodrug can be formed by the replacement of a hydrogen atom in the amine group with a group such as R-carbonyl, RO-carbonyl, NRR′-carbonyl, wherein R and R′ are each independently (C₁-C₁₀)alkyl, (C₃-C₇)cycloalkyl, or benzyl, or R-carbonyl is a natural α-aminoacyl or natural α-aminoacyl-natural α-aminoacyl, —C(OH)C(O)OY wherein Y is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N- or di-N,N—(C₁-C₆)alkylaminoalkyl, or —C(Y₂)Y₃ wherein Y₂ is H or methyl and Y₃ is mono-N- or di-N,N—(C₁-C₆)alkylamino, morpholino, piperidin-1-yl or pyrrolidin-1-yl.

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984).

### Articles of Manufacture

In another embodiment of the invention, an article of manufacture, or “kit”, containing materials useful for the treatment of the diseases and disorders described above is provided. In one embodiment, the kit comprises a container comprising a heterobicyclic pyrazole compound of Formula Ia or Ib, or a stereoisomer, geometric isomer, tautomer, solvate, metabolite, or pharmaceutically acceptable salt or prodrug thereof. The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of Formula Ia or Ib or a formulation thereof which is effective for treating the condition and may have a sterile access port (for example, the container may be an intravenous solution bag or a vial having a stopper pierceable by a hypodermic injection needle). At least one active agent in the composition is a compound of Formula Ia or Ib. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of Formula Ia or Ib can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of Formula Ia or Ib and, if present, the second pharmaceutical formulation. For example, if the kit comprises a first composition comprising a compound of Formula Ia or Ib and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula Ia or Ib, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a compound of Formula Ia or Ib contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of Formula Ia or Ib and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other c-Met inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those

### Example 1

### N-(3-fluoro-4-(3-(piperidin-4-yloxy)-1H-pyrazolo[3,4-b]pyridin-4-yloxv)phenyl)-2-[4-fluorophenyl)-3-oxo-2,3-dihydropyridazine-4-carboxamide dihydrochloride

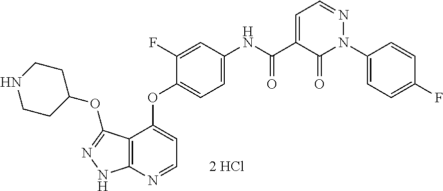

Step A: Preparation of 5-((1-(4-methoxybenzyl)-1H-pyrazol-5-ylamino)methylene)-2,2-dimethyl-1,3-dioxane-4,6-dione: A stirred mixture of triethoxymethane (339 mL, 2037 mmol), and 2,2-dimethyl-1,3-dioxane-4,6-dione (Meldrum's acid) (35.2 g, 244 mmol) was heated to 80° C. for 1 hour. A suspension of 1-(4-methoxybenzyl)-1H-pyrazol-5-amine [41.4 g, 204 mmol; prepared according to the procedure described by Misra, R. N., et al. Bioorg. Med. Chem. Lett. 2003, 13, 1133-1136, except desalting was performed as follows: 1-(4-methoxybenzyl)-1H-pyrazol-5-amine hydrochloride (44 g) was partitioned between MTBE (300 mL) and 1N aqueous sodium hydroxide (300 mL), after separating the phases, the aqueous suspension was re-extracted with MTBE (8×100 mL), followed by drying (Na₂SO₄) the combined organic phases, and concentration in vacuo to obtain the free-based 1-(4-methoxybenzyl)-1H-pyrazol-5-amine (30 g)] in triethoxymethane (339 mL, 2037 mmol) was added at in one portion. The mixture was heated to 80° C. for 18 hours under N₂. After cooling to room temperature, toluene azeotrope (2×200 mL) was utilized to remove ethanol. The resulting suspension was diluted with diethyl ether (500 mL) and filtered to obtain a solid (33.5 g, 46%). ¹H NMR (400 MHz, CDCl₃) δ 11.13 (d, J=13 Hz, 1H), 8.26 (d, J=13 Hz, 1H), 7.50 (d, J=2 Hz, 1H), 7.25 (d, J=9 Hz, 2H), 6.88 (d, J=9 Hz, 2H), 6.21 (d, J=2 Hz, 1H), 5.28 (s, 2H), 3.78 (s, 3H), 1.74 (s, 6H).

Step B: Preparation of 1-(4-methoxybenzyl)-1H-pyrazolo[3,4-b]pyridin-4-ol: 5-((1-(4-Methoxybenzyl)-1H-pyrazol-5-ylamino)methylene)-2,2-dimethyl-1,3-dioxane-4,6-dione (33.5 g, 93.7 mmol) was added in portions as a solid over a 10 minute period to a stirred biphenyl-diphenyl ether eutectic (also called Dowtherm) (100 mL) at 240° C. under N₂. After addition was complete, the mixture was heated at 240° C. for 10 minutes. After cooling to room temperature, the mixture was diluted with hexanes (300 mL), and the hexanes were decanted along with the majority of the Dowtherm. The remaining residue was diluted with diethyl ether (200 mL), and the ether was decanted from the residue and discarded. Lastly the residue was suspended in DCM (100 mL). The stirred suspension was diluted with diethyl ether (300 mL) and filtered. The resulting solid (22.7 g, 91%) was dried under high vacuum. ¹H NMR (400 MHz, DMSO-d6) δ 11.7 (br s, 1H), 8.17 (br s, 1H), 8.08 (s, 1H), 7.20 (d, J=9 Hz, 2H), 6.86 (d, J=9 Hz, 2H), 6.45 (br s, 1H), 5.50 (s, 2H), 3.70 (s, 3H).

Step C: Preparation of 1-(4-methoxybenzyl)-4-(2-fluoro-4-nitrophenoxy)-1H-pyrazolo[3,4-b]pridine: A stirred mixture of 1-(4-methoxybenzyl)-1H-pyrazolo[3,4-b]pyridin-4-ol (22.00 g, 86.18 mmol), cesium carbonate (28.08 g, 86.18 mmol), 1,2-difluoro-4-nitrobenzene (13.71 g, 86.18 mmol) and DMA (100 mL) was heated to 100° C. for 1 hour. After cooling to room temperature, the mixture was partitioned between DCM (500 mL) and water (500 mL). The phases were separated, and the organic phase was washed with water (3×200 mL), dried (Na₂SO₄), filtered, and concentrated in vacuo. The resulting residue was triturated with diethyl ether (100 mL) and hexanes (200 mL) co-solvent, and the resulting powder was filtered. A second crop was obtained after cooling in a −10° C. freezer overnight. The two crops were combined (28 g, 82%). ¹H NMR (400 MHz, CDCl₃) δ 8.45 (d, J=5.5 Hz, 1H), 8.16 (m, 2H), 7.86 (s, 1H), 7.39 (m, 1H), 7.35 (d, J=8.6 Hz, 2H), 6.84 (d, J=8.2 Hz, 2H), 6.48 (d, J=5.5 Hz, 1H), 5.65 (s, 2H), 3.76 (s, 3H). ¹⁹F NMR (376 MHz, CDCl₃) δ −124.2 (m).

Step D: Preparation of 4-(2-fluoro-4-nitrophenoxy)-1H-pyrazolo[3,4-b]pyridine: A stirred mixture of 1-(4-methoxybenzyl)-4-(2-fluoro-4-nitrophenoxy)-1H-pyrazolo[3,4-b]pyridine (27.6 g, 70.0 mmol) and TFA (53.9 mL, 700 mmol) was heated to reflux for 18 hours under N₂. The reaction was allowed to cool to room temperature, and then concentrated in vacuo using toluene (4×100 mL) to azeotrope residual TFA. The residue was diluted with EtOAc (200 mL) and carefully neutralized (pH=8-9) with saturated aqueous NaHCO₃ (100 mL). The biphasic suspension was stirred at room temperature for 30 minutes. The suspension was filtered. The resulting solid was dried by toluene azeotrope (2×200 mL) to obtain the product (18.7 g, 97%). ¹H NMR (DMSO-d6, 400 MHz) δ 13.85 (br s, 1H), 8.40 (m, 2H), 8.15 (m, 1H), 7.91 (s, 1H), 7.66 (m, 1H), 6.65 (m, 1H).

Step E: Preparation of 1-(4-methoxybenzyl)-4-(2-fluoro-4-nitrophenoxy)-3-iodo-1H-pyrazolo[3,4-b]pyridine: Freshly ground potassium hydroxide (10.3 g, 183 mmol) was added to a stirred solution of 4-(2-fluoro-4-nitrophenoxy)-1H-pyrazolo[3,4-b]pyridine (16.7 g, 60.9 mmol) in DMF (250 mL) under N₂ at room temperature followed by iodine (23.2 g, 91.4 mmol). The dark reaction was stirred at room temperature for 18 hours and covered by a foil to minimize light exposure. The reaction was then heated to 50° C. for 3 hours. The reaction was allowed to cool to room temperature. The crude reaction mixture was transferred via cannula into a stirred solution of 1-(chloromethyl)-4-methoxybenzene (11.1 g, 70 7 mmol) in DMF (100 mL) which was cooled in an ice bath under N₂. The reaction was allowed to stir for 18 hours under N₂ at room temperature. The mixture was then diluted with DCM (1 L) and washed with 5% aqueous Na₂S₂O₃ (1 L). The aqueous phase was back-extracted with DCM (2×200 mL). The combined organic phases were washed with water (4×500 mL), dried (Na₂SO₄), filtered, and concentrated in vacuo. The resulting residue was triturated with DCM (100 mL), and the undissolved solid removed by filtration. The filtrate was purified by Biotage Flash 65, eluting with 10% EtOAc/hexanes, 20% EtOAc/hexanes, then 30% EtOAc/hexanes to elute the desired product. The product was obtained as a solid (16.6 g, 47%). ¹H NMR (400 MHz, CDCl₃) δ 8.42 (d, J=6 Hz, 1H), 8.16 (m, 2H), 7.38 (d, J=9 Hz, 2H), 7.34 (m, 1H), 6.84 (d, J=9 Hz, 2H), 6.36 (d, J=6 Hz, 1H), 5.63 (s, 2H), 3.77 (s, 3H).

Step F: Preparation of 4-(1-(4-methoxybenzyl)-3-iodo-1H-pyrazolo[3,4-b]piperdin-4-yloxy)-3-fluorobenzenamine: A stirred mixture of 1-(4-methoxybenzyl)-4-(2-fluoro-4-nitrophenoxy)-3-iodo-1H-pyrazolo[3,4-b]pyridine (10.4 g, 20.0 mmol), stannous chloride-dihydrate (22.6 g, 100.0 mmol), and absolute EtOH (200 mL) was heated to 65° C. for 1.5 hours under N₂. After cooling to room temperature, the reaction was concentrated in vacuo, and then diluted with DCM (100 mL) and water (100 mL). Aqueous 2N NaOH was added until the pH of the aqueous phase was in the 11-12 range. The biphasic suspension was filtered through a pad of celite, rinsing with DCM (3×100 mL). The filtered biphase was separated, and the aqueous phase was re-extracted with DCM (3×75 mL). The combined organic phases were dried (Na₂SO₄), filtered, and concentrated. Yield: 7.90 g, 78%. The product was used in the next step without purification. ¹H NMR (400 MHz, CDCl₃) δ 8.30 (d, J=6 Hz, 1H), 7.35 (d, J=9 Hz, 2H), 7.03 (t, J=9 Hz, 1H), 6.83 (d, J=9 Hz, 2H), 6.53 (m, 2H), 6.24 (m, 1H), 5.61 (s, 2H), 3.81 (s, 2H), 3.76 (s, 3H).

Step G: Preparation of (E)-2-(2-(4-fluorophenyphydrazono)acetaldehyde: A mixture of 1-(4-fluorophenyl)hydrazine hydrochloride (5.0 g, 30.75 mmol), water (20 mL), and acetic acid (20 mL) was added with stirring to a 40% aqueous solution of glyoxal (17.6 mL, 153.8 mmol) over 20 minutes. Stirring was continued for 2 hours and, the mixture was then filtered. The precipitate was washed with water and dried to afford the desired product (5.0 g, 98%). ¹H NMR (400 MHz, CDCl₃) δ 9.56 (d, 1H), 8.63 (br s, 1H), 7.24 (m, 1H), 7.16 (m, 2H), 7.06 (m, 2H);¹⁹F NMR (376 MHz, CDCl₃) δ−120.3. LRMS (ESI pos) m/e 151.1 (M−16).

Step H: Preparation of (E)-5-(2-(2-(4-fluorophenyl)hydrazono)ethylidene)-2,2-dimethyl-1,3-dioxane-4,6-dione: A suspension of dioxan-dione (1.44 g, 10.0 mmol) and (E)-2-(2-(4-fluorophenyphydrazono)acetaldehyde (1.66 g, 10.0 mmol) in toluene (15 mL) was treated with acetic acid (5 drops) and piperidine (5 drops). The reaction mixture was then stirred at room temperature for 17 hours. The precipitated condensation product was filtered and thoroughly washed with light petroleum to afford the desired product (2.87 g, 98%). ¹H NMR (400 MHz, CD₃OD/CDCl₃) δ 8.72 (d, 1H), 8.24 (d, 1H), 7.32 (m, 2H), 7.08 (t, 2H), 1.76 (s, 6H); ¹⁹F NMR (376 MHz, CDCl₃) δ−119.1.

Step I: Preparation of 2-(4-fluorophenyl)-3-oxo-2,3-dihydropyridazine-4-carboxylic acid: A mixture of (E)-5-(2-(2-(4-fluorophenyphydrazono)ethylidene)-2,2-dimethyl-1,3-dioxane-4,6-dione (0.60 g, 2.05 mmol) and sodium methoxide (0.133 g, 2.46 mmol) in MeOH (10 mL) was heated under reflux for 15 hours. The salt was treated with cold 1 N HCl solution, extracted with DCM, dried over MgSO₄, and concentrated to afford the desired product (0.42 g, 87%). ¹H NMR (400 MHz, CDCl₃) δ 13.57 (br s, 1H), 8.29 (m, 2H), 7.63 (m, 2H), 7.24 (m, 2H); ¹⁹F NMR (376 MHz, CDCl₃) δ−110.7. LRMS (ESI pos) m/e 235.1 (M+1).

Step J: Preparation of tert-butyl 4-(4-(4-amino-2-fluorophenoxy)-1-(4-methoxybenzyl)-1H-pyrazolo[3,4-b]pyridin-3-yloxy)piperidine-1-carboxylate : A mixture of 4-(1-(4-methoxybenzyl)-3-iodo-1H-pyrazolo[3,4-b]pyridin-4-yloxy)-3-fluorobenzenamine (1.0 g, 2.94 mmol), tert-butyl 4-hydroxypiperidine-1-carboxylate (12.3 g, 61.2 mmol), 1,10-phenanthroline (0.37 g, 2.04 mmol), CuI (0.39 g, 2.04 mmol), and KF on Al₂O₃ (2.08 g, 14.3 mmol, 40% Wt) was heated in toluene (20 mL) at 110° C. for 17 hours. After cooling to room temperature, the reaction mixture was filtered through a pad of celite with hexane and then DCM. The filtrate was concentrated to give the product which was flash chromatographed (SiO2, 0 to 1% MeOH in CH₂Cl₂) to remove the excess alcohol. The product was obtained along with tert-butyl 4-hydroxypiperidine-1-carboxylate as a starting material. LRMS (APCI pos) m/e 564.1 (M+1).

Step K: Preparation of tert-butyl 4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidine-1-carboxylate: A mixture of 2-(4-fluorophenyl)-3-oxo-2,3-dihydropyridazine-4-carboxylic acid (76 mg, 0.33 mmol), EDCI (120 mg, 0.65 mmol), and HOBt (88 mg, 0.65 mmol) in DMF (2 mL) was stirred at room temperature for 10 minutes. tert-Butyl 4-(4-(4-amino-2-fluorophenoxy)-1-(4-methoxybenzyl)-1H-pyrazolo[3,4-b]pyridin-3-yloxy)piperidine-1-carboxylate (0.13 g, 0.11 mmol, 47% purity with a mixture of tert-butyl 4-hydroxypiperidine-1-carboxylate) was added, followed by Et₃N (0.091 mL, 0.65 mmol). After stirring for 2 hours, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material. The crude material was purified by silica gel flash column chromatography (1% MeOH in CH₂Cl₂) to afford the desired product (85 mg, 100%). LRMS (ACPI pos) m/e 780.2 (M+1).

Step L: Preparation of N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide: A mixture of tert-butyl 4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidine-1-carboxylate (0.085 g, 0.109 mmol) and TFA (2.52 mL, 32.7 mmol) in CH₂Cl₂ (5 mL) was stirred at room temperature for 6 hours. The reaction mixture was concentrated under reduced pressure using toluene to azeotrope to afford the desired product (77 mg, TFA salt, 81%). LRMS (ESI pos) m/e 680.2

Step M: Preparation of N-(3-fluoro-4-(3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide dihydrochloride: A mixture of N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (0.050 g, 0.074 mmol) and TFA (3.4 mL, 44.1 mmol) was placed in a vial and heated at 65° C. for 5 days. The reaction mixture was concentrated under reduced pressure using toluene to azeotrope. The crude was treated with EtOAc, washed with saturated aqueous NaHCO₃ and brine, dried over MgSO₄, and concentrated to give product which was flash chromatographed (SiO₂, 90:10=DCM: 7N NH₃ in MeOH). The product was treated with 2 N HCl (5 drops) in a mixture of Et₂O and MeOH (5 mL, 4:1 ratio). After 10 minutes stirring, the solvent was removed under reduced pressure to give the HCl salt product (9.7 mg, 34%) which was rinsed with Et₂O. LRMS (ESI pos) m/e 560.0 (M+1). ¹H-NMR (400 MHz, CDCl₃) δ 11.81 (s, 1H), 8.41 (d, 1H), 8.25 (dd, 2H), 7.94 (dd, 1H), 7.60 (m, 2H), 7.38 (d, 1H), 7.24 (m, 3H), 6.25 (d, 1H), 4.96 (m, 1H), 3.15 (m, 2H), 2.76 (t, 2H), 2.13 (m, 2H), 1.77 (m, 2H); ¹⁹F NMR (376 MHz, CDCl₃) δ−111.4, −126.6.

### Example 2

### N-3-fluoro-4-(3-(1-methylpiperidin-4-yloxy)-1H-pyrazolo[3 4-b]pyridin-4-yloxy)phenyl)-2-(4-fluorophenyl)-3-oxo-2,3-dihydropyridazine-4-carboxamide dihydrochloride

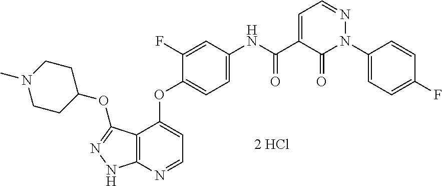

Step A: Preparation of N-(3-fluoro-4-(1-(4-methoxybenzyl)-3-(1-methylpiperidin-4-yloxy)-1H-pyrazolo[3,4-b]pyridin-4-yloxy)phenyl)-2-(4-fluorophenyl)-3-oxo-2,3-dihydropyridazine-4-carboxamide: NaBH(OAc)₃ (0.26 g, 1.24 mmol) was added to a THF solution (5 mL) of N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (0.280 g, 0.41 mmol; prepared as in Example 1, Step K), formaldehyde (0.1 g, 1.24 mmol, 37% Wt in water), and acetic acid (2.4 μL, 0.041 mmlol) at room temperature. The reaction mixture was stirred for 1 hour at room temperature. The mixture was treated with water (10 mL), extracted with EtOAc, dried over MgSO₄, and concentrated under reduced pressure to give the crude material. The crude material was purified by silica gel flash column chromatography (3% MeOH in CH₂Cl₂) to afford the desired product (0.17 g, 60%). LRMS (ESI pos) m/e 694.1 (M+1).

Step B: Preparation of N-(3-fluoro-4-(3-(1-methylpiperidin-4-yloxy)-1H-pyrazolo[3,4-b]pyridin-4-yloxy)phenyl)-2-(4-fluorophenyl)-3-oxo-2,3-dihydropyridazine-4-carboxamide dihydrochloride: Prepared from N-(3-fluoro-4-(1-(4-methoxybenzyl)-3-(1-methylpiperidin-4-yloxy)-1H-pyrazolo[3,4-b]pyridin-4-yloxy)phenyl)-2-(4-fluorophenyl)-3-oxo-2,3-dihydropyridazine-4-carboxamide according to the procedure of Example 1, Step M. Purified by silica gel flash column chromatography (SiO₂, aqueous 30% NH₄OH:MeOH:dichloromethane=0.1:4.9:95) to provide the product as a solid (70 mg, 50%). LRMS (ESI pos) m/e 574.1 (M+1). ¹H NMR (400 MHz, CD₃OD/CDCl₃) δ 8.37 (d, 1H), 8.31 (d, 1H), 8.21 (d, 1H), 8.0 (dd, 1H), 7.67 (m, 2H), 7.44 (d, 1H), 7.28 (m, 3H), 6.27 (d, 1H), 4.91 (m, 1H), 2.74 (m, 2H), 2.40 (m, 2H), 2.29 (s, 3H), 2.10 (m, 2H), 1.96 (m, 2H); ¹⁹F NMR (376 MHz, CD₃OD/CDCl₃) δ−114.0, −129.0.

### Example 3

### N-(4-(3-(1-ethylpiperidin-4-yloxy)-1H-pyrazolo[3,4-b]pyridin-4-yloxy)-3-fluorophenyl)-2-[4-fluorophenyl)-3-oxo-2,3-dihydropyridazine-4-carboxamide dihydrochloride

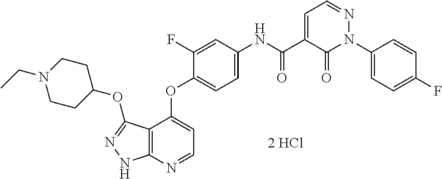

Step A: Preparation of N-(4-(3-(1-ethylpiperidin-4-yloxy)-1H-pyrazolo[3,4-b]pyridin-4-yloxy)-3-fluorophenyl)-2-(4-fluorophenyl)-3-oxo-2,3-dihydropyridazine-4-carboxamide dihydrochloride: Prepared by a 2-step process from N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide and acetaldehyde according to the procedure of Example 2, Steps A and B to afford the desired product (4.5 mg, 35% yield for 2 step process). LRMS (ESI pos) m/e 588.1 (M+1). ¹H NMR (400 MHz, CD₃OD) δ 8.31 (d, 1H), 8.25 (d, 1H), 8.19 (d, 1H), 7.98 (dd, 1H), 7.63 (m, 2H), 7.42 (d, 1H), 7.26 (q, 3H), 6.25 (d, 1H), 4.88 (m, 1H), 2.79 (m, 2H), 2.40-2.48 (m, 4H), 2.08 (m, 2H), 1.91 (m, 2H), 1.07 (t, 3H); ¹⁹F NMR (376 MHz, CD₃OD) δ−114.7, −129.8.

### Example 4

### N-(4-(3-(1-acetylpiperidin-4-yloxy)-1H-pyrazolo[3,4-b]pyridin-4-yloxy)-3-fluorophenyl)-2-(4-fluorophenyl)-3-oxo-2,3-dihydropyridazine-4-carboxamide hydrogen chloride

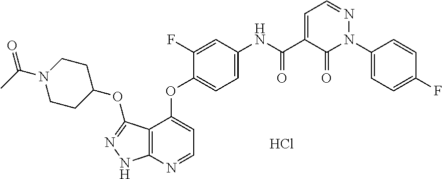

Step A: Preparation of N-(4-(3-(1-acetylpiperidin-4-yloxy)-1-(4-methoxybenzyl)-1H-pyrazolo[3,4-b]pyridin-4-yloxy)-3-fluorophenyl)-2-(4-fluorophenyl)-3-oxo-2,3-dihydropyridazine-4-carboxamide: A mixture of acetic acid (27 mg, 0.44 mmol), EDCI (85 mg, 0.44 mmol), and HOBt (68 mg, 0.44 mmol) in DMF (2 mL) was stirred at room temperature for 10 minutes. N-(3-Fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (50 mg, 0.074 mmol; prepared as in Example 1, Step K) was added, followed by Et₃N (0.062 mL, 0.44 mmol). After stirring for 1 hour, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material. The material was purified by silica gel flash column chromatography (1% MeOH in CH₂Cl₂) to afford the desired product (48 mg, 90%). LRMS (ESI pos) m/e 722.1 (M+1).

Step B: Preparation of N-(4-(3-(1-acetylpiperidin-4-yloxy)-1H-pyrazolo[3,4-b]pyridin-4-yloxy)-3-fluorophenyl)-2-(4-fluorophenyl)-3-oxo-2,3-dihydropyridazine-4-carboxamide hydrogen chloride: Prepared from N-(4-(3-(1-acetylpiperidin-4-yloxy)-1-(4-methoxybenzyl)-1H-pyrazolo[3,4-b]pyridin-4-yloxy)-3-fluorophenyl)-2-(4-fluorophenyl)-3-oxo-2,3-dihydropyridazine-4-carboxamide according to the procedure of Example 2, Step B. Purified by silica gel flash column chromatography (SiO₂, aqueous 30% NH₄OH:MeOH:DCM=0.1:4.9:95) to provide the product as a solid (31 mg, 78%). LRMS (ESI pos) m/e 602.1 (M+1). ¹H NMR (400 MHz, CD₃OD) δ 8.31 (d, 1H), 8.25 (d, 1H), 8.21 (d, 1H), 7.97 (dd, 1H), 7.63 (dd, 2H), 7.41 (d, 1H), 7.25 (t, 3H), 6.30 (d, 1H), 5.05 (m, 1H), 3.74 (m, 2H), 3.46 (m, 2H), 2.06 (s, 3H), 1.97 (m, 2H), 1.86 (m, 1H), 1.77 (m, 1H); ¹⁹F NMR (376 MHz, CD₃OD) δ−114.8, −130.1.

### Example 5

### N-3-fluoro-4-(3-(1-(2-fluoroethyl)piperidin-4-yloxy)-1H-pyrazolo[3,4-b]pyridin-4-yloxy)phenyl)-2-(4-fluorophenyl)-3-oxo-2,3-dihydropyridazine-4-carboxamide

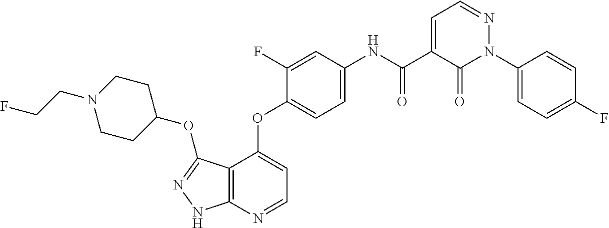

Step A: Preparation of N-(3-fluoro-4-(3-(1-(2-fluoroethyl)piperidin-4-yloxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide: 1-Bromo-2-fluoroethane was added to a mixture of N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (40 mg, 0.059 mmol; prepared as in Example 1, Step K) and NaH (2.8 mg, 0.12 mmol) in DMF (2 mL) at 0° C. The reaction mixture was allowed to warm to room temperature. After stirring for three hours, the reaction was heated to 40° C. for 17 hours and then stirred at room temperature for 3 days. The mixture was quenched with saturated aqueous NH₄Cl, extracted with EtOAc, washed with brine, dried over MgSO₄, and concentrated under reduced pressure to give the crude material that was purified by silica gel flash column chromatography (2% MeOH in CH₂Cl₂) to afford the desired product (3.8 mg, 9%). LRMS (ESI pos) m/e 726.1 (M+1).

Step B: Preparation of N-(3-fluoro-4-(3-(1-(2-fluoroethyl)piperidin-4-yloxy)-1H-pyrazolo[3,4-b]pyridin-4-yloxy)phenyl)-2-(4-fluorophenyl)-3-oxo-2,3-dihydropyridazine-4-carboxamide: Prepared from N-(3-fluoro-4-(3-(1-(2-fluoroethyl)piperidin-4-yloxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide according to the procedure of Example 2, Step B. Purified by silica gel flash column chromatography (SiO₂, aqueous 30% NH₄OH:MeOH:dichloromethane=0.2:4.8:95) to provide the product as a solid (2 mg, 63%). LRMS (ESI pos) m/e 606.1 (M+1). ¹H NMR (400 MHz, CD₃OD/CDCl₃) δ 8.40 (d, 1H), 8.32 (d, 1H), 8.22 (d, 1H), 7.99 (dd, 1H), 7.65 (dd, 2H), 7.44 (d, 1H), 7.28 (t, 3H), 6.28 (d, 1H), 4.93 (m, 1H), 4.64, (t, 1H), 4.52 (t, 1H), 2.85 (m, 2H), 2.77 (t, 1H), 2.70 (t, 1H), 2.52 (m, 2H), 2.11 (m, 2H), 1.99 (m, 2H); ¹⁹F NMR (376 MHz, CD₃OD/CDCl₃) δ−113.2, −128.4, −219.9.

### Example 6

### N-(3-fluoro-4-(3-(1-(2-hydroxyethyl)piperidin-4-yloxy)-1H-pyrazolo[3,4-b]pyridin-4-yloxy)nhenyl)-2-(4-fluorophenyl)-3-oxo-2,3-dihydropyridazine-4-carboxamide dihydrochloride

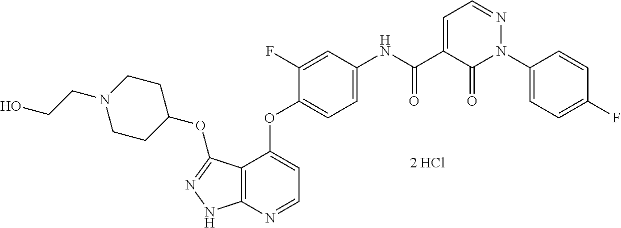

Step A: Preparation of 2-(4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidin-1-yl)ethyl acetate: 2-Bromoethyl acetate was added to a mixture of N-(3-fluoro-4-(1-(4-methoxybenzyl)-3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (40 mg, 0.059 mmol; prepared as in Example 1, Step K) and NaH (4.2 mg, 0.18 mmol) in DMF (2 mL) at room temperature. After three days stirring, the mixture was treated with EtOAc (300 mL), washed with saturated aqueous NH₄Cl and brine, dried over MgSO₄, and concentrated under reduced pressure to give the crude material. The crude material was purified by silica gel flash column chromatography (2% MeOH in CH₂Cl₂) to afford the desired product (9 mg, 20%). LRMS (ESI pos) m/e 766.2 (M+1).

Step B: Preparation of N-(3-fluoro-4-(3-(1-(2-hydroxyethyl)piperidin-4-yloxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide: 1N aqueous lithium hydroxide (3 drops) was added to a solution of 2-(4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidin-1-yl)ethyl acetate (9 mg, 0.12 mmol) in THF-MeOH (4:1 ratio, 5 mL) at room temperature. The reaction mixture was stirred for 20 minutes. 1N aqueous HCl (3 drops) was added to the mixture, and then the solvent was removed under reduced pressure to give the product salt, which was treated with further purification. LRMS (ESI pos) m/e 724.1 (M+1).

Step C: Preparation of N-(3-fluoro-4-(3-(1-(2-hydroxyethyl)piperidin-4-yloxy)-1H-pyrazolo[3,4-b]pyridin-4-yloxy)phenyl)-2-(4-fluorophenyl)-3-oxo-2,3-dihydropyridazine-4-carboxamide dihydrochloride: Prepared from N-(3-fluoro-4-(3-(1-(2-hydroxyethyl)piperidin-4-yloxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide according to the procedure of Example 2, Step B. Purified by silica gel flash column chromatography (SiO₂, aqueous 30% NH₄OH:MeOH:DCM=0.5:6.5:93) to provide the product as a solid (0.8 mg, 11%). LRMS (ESI pos) m/e 604.1 (M+1).

### Example 7

### N-(3-fluoro-4-(3-((1-methylpiperidin-4-yl)methoxy)-1H-pyrazolo[3,4-b]pyridin-4-yloxy)phenyl)-2-(4-fluorophenyl)-3-oxo-2,3-dihydropyridazine-4-carboxamide dihydrochloride

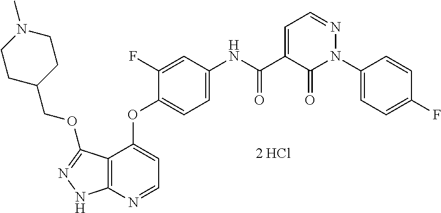

Step A: Preparation of tert-butyl 4-((4-(4-amino-2-fluorophenoxy)-1-(4-methoxybenzyl)-1H-pyrazolo[3,4-b]pyridin-3-yloxy)methyl)piperidine-1-carboxylate: Prepared from 4-(1-(4-methoxybenzyl)-3-iodo-1H-pyrazolo[3,4-b]pyridin-4-yloxy)-3-fluorobenzenamine (0.5 g, 1.02 mmol; prepared as in Example 1, Step E) and tert-butyl 4-(hydroxymethyl)piperidine-1-carboxylate (3.82 g, 17.75 mmol) according to the procedure of Example 1, Step I. Purified by silica gel flash column chromatography (SiO₂, 0 to 1% MeOH in DCM). The product was obtained along with tert-butyl 4-(hydroxymethyl)piperidine-1-carboxylate as a starting material.

Step B: Preparation of N-(3-fluoro-4-(1-(4-methoxybenzyl)-3-(piperidin-4-ylmethoxy)-1H-pyrazolo[3,4-b]pyridin-4-yloxy)phenyl)-2-(4-fluorophenyl)-3-oxo-2,3-dihydropyridazine-4-carboxamide: Prepared by a 2-step process from tert-butyl 4-((4-(4-amino-2-fluorophenoxy)-1-(4-methoxybenzyl)-1H-pyrazolo[3,4-b]pyridin-3-yloxy)methyl)piperidine-1-carboxylate, 2-(4-fluorophenyl)-3-oxo-2,3-dihydropyridazine-4-carboxylic acid, and TFA according to the procedure of Example 1, Steps J and K. Purified by silica gel flash column chromatography (SiO₂, 2% MeOH in DCM) to afford the desired product (0.389 g, 55% yield for 3 step process). LRMS (APCI pos) m/e 694.2 (M+1).

Step C: Preparation of N-(3-fluoro-4-(3-((1-methylpiperidin-4-yl)methoxy)-1H-pyrazolo[3,4-b]pyridin-4-yloxy)phenyl)-2-(4-fluorophenyl)-3-oxo-2,3-dihydropyridazine-4-carboxamide dihydrochloride: Prepared by a 2-step process from N-(3-fluoro-4-(1-(4-methoxybenzyl)-3-(piperidin-4-ylmethoxy)-1H-pyrazolo[3,4-b]pyridin-4-yloxy)phenyl)-2-(4-fluorophenyl)-3-oxo-2,3-dihydropyridazine-4-carboxamide, formaldehyde, and TFA according to the procedure of Example 2, Steps A and B. Purified by silica gel flash column chromatography (SiO₂, 95:4.8:0.2=dichloromethane:MeOH: aqueous 30% NH₄OH) to afford the desired product (0.12 g, 43% yield for 2 step process). LRMS (ESI pos) m/e 588.1 (M+1). ¹H NMR (400 MHz, CD₃OD/CDCl₃) δ 8.41 (d, 1H), 8.28 (d, 1H), 8.18 (d, 1H), 7.95 (dd, 1H), 7.61 (m, 2H), 7.41 (d, 1H), 7.26 (t, 3H), 6.21 (d, 1H), 4.26 (d, 2H), 2.90 (m, 2H), 2.68 (m, 1H), 2.27 (s, 3H), 2.05 (t, 2H), 1.89 (m, 2H), 1.45 (m, 2H); ¹⁹F NMR (376 MHz, CD₃OD) δ−114.7, −129.7.

### Example 8

### N-(4-(3-((1-ethylpiperidin-4-yl)methoxy)-1H-pyrazolo[3,4-b]pyridin-4-yloxy)-3-fluorophenyl)-2-(4-fluorophenyl)-3-oxo-2,3-dihydropyridazine-4-carboxamide dihydrochloride

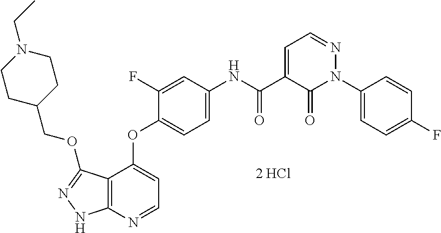

Step A: Preparation of N-(4-(3-((1-ethylpiperidin-4-yl)methoxy)-1H-pyrazolo[3,4-b]pyridin-4-yloxy)-3-fluorophenyl)-2-(4-fluorophenyl)-3-oxo-2,3-dihydropyridazine-4-carboxamide dihydrochloride: Prepared by a 2-step process from N-(3-fluoro-4-(1-(4-methoxybenzyl)-3-(piperidin-4-ylmethoxy)-1H-pyrazolo[3,4-b]pyridin-4-yloxy)phenyl)-2-(4-fluorophenyl)-3-oxo-2,3-dihydropyridazine-4-carboxamide (prepared as in Example 7, Step B), acetaldehyde, and TFA according to the procedure of Example 2, Steps A and B. Purified by silica gel flash column chromatography (SiO₂, 95:4.9:0.1=dichloromethane:MeOH: aqueous 30% NH₄OH) to afford the desired product (13.5 mg, 80% yield). LRMS (ESI pos) m/e 602.0 (M+1). ¹H NMR (400 MHz, CD₃OD/CDCl₃) δ 8.39 (d, 1H), 8.32 (d, 1H), 8.22 (d, 1H), 7.99 (dd, 1H), 7.66 (m, 2H), 7.43 (d, 1H), 7.28 (t, 3H), 6.27 (d, 1H), 4.25 (d, 2H), 3.12 (m, 2H), 2.94 (m, 1H), 2.58 (q, 2H), 2.23 (t, 2H), 1.94 (m, 2H), 1.51 (m, 2H), 1.42 (t, 3H); ¹⁹F NMR (376 MHz, CD₃OD) δ−113.5, −128.6.

### Example 9

### N-(3-fluoro-4-(3-((1-(2-hydroxyacetyl)piperidin-4-yl)methoxy)-1H-pyrazolo[3,4-b]pyridin-4-yloxy)phenyl-2-(4-fluorophenyl)-3-oxo-2,3-dihydropyridazine-4-carboxamide hydrogen chloride

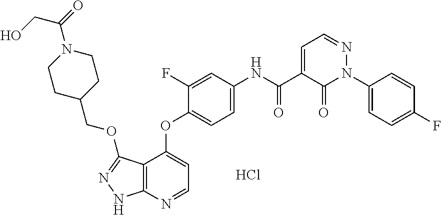

Step A: Preparation of N-(3-fluoro-4-(3-((1-(2-hydroxyacetyl)piperidin-4-yl)methoxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide: A mixture of 2-hydroxyacetic acid (22 mg, 0.29 mmol), EDCI (55 mg, 0.29 mmol), and HOBt-H₂O (44 mg, 0.29 mmol) in DMF (1 mL) was stirred at room temperature for 1 minute. N-(3-Fluoro-4-(1-(4-methoxybenzyl)-3-(piperidin-4-ylmethoxy)-1H-pyrazolo[3,4-b]pyridin-4-yloxy)phenyl)-2-(4-fluorophenyl)-3-oxo-2,3-dihydropyridazine-4-carboxamide (40 mg, 0.058 mmol; prepared as in Example 7, Step B) was added, followed by Et₃N (0.04 mL, 0.29 mmol). After stirring for 30 minutes, the reaction mixture was diluted with EtOAc and washed with saturated aqueous NH₄Cl, saturated aqueous NaHCO₃, and brine. The organic layer was dried over MgSO₄ and concentrated under reduced pressure to give the crude material. The crude material was treated with aqueous 1 N lithium hydroxide (3 drops) to remove the acetyl group as a double addition byproduct in THF-MeOH (5:1 ratio, 6 mL) at room temperature. After 10 minutes of stirring, the mixture was treated with aqueous 1N HCl (3 drops) and EtOAc (100 mL), washed with saturated aqueous NaHCO₃ and brine, dried over MgSO₄, and concentrated under reduced pressure to give the crude material. The crude material was directly used in the next step without further purification. LRMS (ESI pos) m/e 752.1 (M+1).

Step B: Preparation of N-(3-fluoro-4-(3-((1-(2-hydroxyacetyl)piperidin-4-yl)methoxy)-1H-pyrazolo[3,4-b]pyridin-4-yloxy)phenyl)-2-(4-fluorophenyl)-3-oxo-2,3-dihydropyridazine-4-carboxamide hydrogen chloride: Prepared from N-(3-fluoro-4-(3-((1-(2-hydroxyacetyl)piperidin-4-yl)methoxy)-1-(4-methoxybenzyl)-1H-pyrazolo[3,4-b]pyridin-4-yloxy)phenyl)-2-(4-fluorophenyl)-3-oxo-2,3-dihydropyridazine-4-carboxamide according to the procedure of Example 1, Step L. Purified by silica gel flash column chromatography (SiO2, 3:97=7N NH₃ in MeOH:dichloromethane) to provide the product as a solid (15 mg, 41% for 2 step process). LRMS (ESI pos) m/e 632.0 (M+1). ¹H NMR (400 MHz, CD₃OD) δ 8.31 (d, 1H), 8.26 (d, 1H), 8.21 (d, 1H), 7.96 (dd, 1H), 7.63 (m, 2H), 7.40 (d, 1H), 7.25 (m, 3H), 6.30 (d, 1H), 4.16 (m, 4H), 3.70 (d, 1H), 3.0 (t, 1H), 2.69 (t, 1H), 2.08 (m, 1H), 1.83 (m, 2H), 1.24 (m, 3H); ¹⁹F NMR (376 MHz, CD₃OD) δ−114.7, −130.1.

### Example 10

### N-(3-fluoro-4-(3-(1-(2-hydroxyacetyl)piperidin-4-yloxy)-1H-pyrazolo[3,4-b]pyridin-4-yloxy)phenyl)-2-(4-fluorophenyl)-3-oxo-2,3-dihydropyridazine-4-carboxamide hydrogen chloride

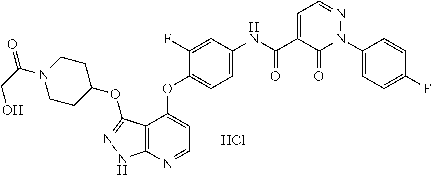

Step A: Preparation of N-(3-fluoro-4-(3-(1-(2-hydroxyacetyl)piperidin-4-yloxy)-1H-pyrazolo[3,4-b]pyridin-4-yloxy)phenyl)-2-(4-fluorophenyl)-3-oxo-2,3-dihydropyridazine-4-carboxamide hydrogen chloride: Prepared from N-(3-fluoro-4-(3-(piperidin-4-yloxy)-1H-pyrazolo[3,4-b]pyridin-4-yloxy)phenyl)-2-(4-fluorophenyl)-3-oxo-2,3-dihydropyridazine-4-carboxamide (prepared as in Example 1, Step L) according to the procedure of Example 9, Step A except that the reaction was not treated with LiOH. Purified by silica gel flash column chromatography (SiO₂, 3 to 5% 7N NH₃—MeOH in DCM) to provide the product as a solid (25 mg, 76%). LRMS (APCI pos) m/e 618.0 (M+1). ¹H NMR (400 MHz, CDCl₃) δ 11.82 (s, 1H), 10.07 (s, 1H), 8.41 (d, 1H), 8.30 (d, 1H), 8.24 (d, 1H), 7.95 (dd, 1H), 7.60 (m, 2H), 7.37 (d, 1H), 7.17-7.27 (m, 3H), 6.30 (d, 1H), 5.15 (m, 1H), 4.17 (d, 2H), 3.75-3.81 (m, 2H), 3.65 (t, 1H), 3.51 (m, 1H), 3.22 (m, 1H), 2.0 (m, 4H); ¹⁹F NMR (376 MHz, CDCl₃) δ−111.4, −127.0.

### Example 11

### N-(3-fluoro-4-(3-(2-morpholinoethoxy)-1H-pyrazolo[3,4-b]pyridin-4-yloxy)phenyl)-2-(4-fluorophenyl)-3-oxo-2,3-dihydropyridazine-4-carboxamide dihydrochloride

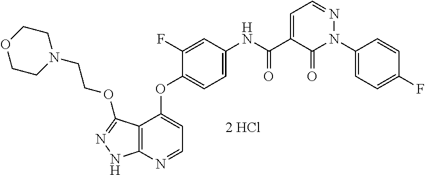

Step A: Preparation of 3-fluoro-4-(1-(4-methoxybenzyl)-3-(2-morpholinoethoxy)-1H-pyrazolo[3,4-b]pyridin-4-yloxy)aniline: Prepared from 4-(1-(4-methoxybenzyl)-3-iodo-1H-pyrazolo[3,4-b]pyridin-4-yloxy)-3-fluorobenzenamine (prepared as in Example 1, Step E) according to the procedure of Example 1, Step I. Purified by silica gel flash column chromatography (SiO₂, 2% MeOH in DCM) to provide the product as a solid (147 mg, 49%). LRMS (ESII pos) m/e 494.1 (M+1).

Step B: Preparation of N-(3-fluoro-4-(1-(4-methoxybenzyl)-3-(2-morpholinoethoxy)-1H-pyrazolo[3,4-b]pyridin-4-yloxy)phenyl)-2-(4-fluorophenyl)-3-oxo-2,3-dihydropyridazine-4-carboxamide: Prepared from 3-fluoro-4-(1-(4-methoxybenzyl)-3-(2-morpholinoethoxy)-1H-pyrazolo[3,4-b]pyridin-4-yloxy)aniline according to the procedure of Example 1, Step J. Purified by silica gel flash column chromatography (SiO₂, 1% MeOH in DCM) to provide the product as a solid (108 mg, 64%). LRMS (ESI pos) m/e 710.1 (M+1).

Step C: Preparation of N-(3-fluoro-4-(3-(2-morpholinoethoxy)-1H-pyrazolo[3,4-b]pyridin-4-yloxy)phenyl)-2-(4-fluorophenyl)-3-oxo-2,3-dihydropyridazine-4-carboxamide dihydrochloride: Prepared from N-(3-fluoro-4-(1-(4-methoxybenzyl)-3-(2-morpholinoethoxy)-1H-pyrazolo[3,4-b]pyridin-4-yloxy)phenyl)-2-(4-fluorophenyl)-3-oxo-2,3-dihydropyridazine-4-carboxamide according to the procedure of Example 1, Step L. Purified by silica gel flash column chromatography (SiO2, 5:95=7N NH₃ in MeOH:dichloromethane) to provide the product as a solid (50 mg, 66%). LRMS (ESI pos) m/e 590.0 (M+1). ¹H NMR (400 MHz, CD₃OD/CDCl₃) δ 8.42 (d, 1H), 8.38 (d, 1H), 8.34 (d, 1H), 8.07 (dd, 1H), 7.66 (m, 2H), 7.50 (d, 1H), 7.40 (t, 1H), 7.30 (t, 2H), 6.50 (d, 1H), 4.93 (m, 2H), 3.98 (m, 4H), 3.72 (m, 4H); ¹⁹F NMR (376 MHz, CD₃OD/CDCl₃) δ−112.7, −128.6.

### Example 12

### S—N-(4-(3-(2,3-dihydroxypropyl)-1H-pyrazolo[3,4-b]pyridin-4-yloxy)-3-fluorophenyl)-2-(4-fluorophenyl)-3-oxo-2,3-dihydropyridazine-4-carboxamide hydrogen chloride

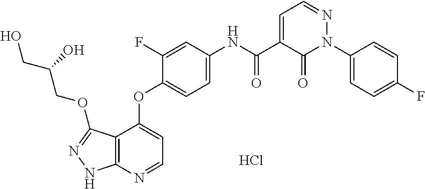

Step A: Preparation of (R)-4-(3-((2,2-dimethyl-1,3-dioxolan-4-yl)methoxy)-1-(4-methoxybenzyl)-1H-pyrazolo[3,4-b]pyridin-4-yloxy)-3-fluoroaniline: Prepared from 4-(1-(4-methoxybenzyl)-3-iodo-1H-pyrazolo[3,4-b]pyridin-4-yloxy)-3-fluorobenzenamine (prepared as in Example 1, Step E) according to the procedure of Example 1, Step I. Purified by silica gel flash column chromatography (SiO₂, 0.5% MeOH in DCM) to provide the product as a solid (96 mg, 60%). LRMS (ESII pos) m/e 495.1 (M+1).

Step B: Preparation of (R)—N-(4-(3-((2,2-dimethyl-1,3-dioxolan-4-yl)methoxy)-1-(4-methoxybenzyl)-1H-pyrazolo[3,4-b]pyridin-4-yloxy)-3-fluorophenyl)-2-(4-fluorophenyl)-3-oxo-2,3-dihydropyridazine-4-carboxamide: Prepared from (R)-4-(3-((2,2-dimethyl-1,3-dioxolan-4-yl)methoxy)-1-(4-methoxybenzyl)-1H-pyrazolo[3,4-b]pyridin-4-yloxy)-3-fluoroaniline according to the procedure of Example 1, Step J. Purified by silica gel flash column chromatography (SiO₂, 2:1=hexane:ethyl acetate) to provide the product as a solid (76 mg, 55%). LRMS (APCI pos) m/e 711.3 (M+1).

Step C: Preparation of (S)—N-(4-(3-(2,3-dihydroxypropoxy)-1H-pyrazolo[3,4-b]pyridin-4-yloxy)-3-fluorophenyl)-2-(4-fluorophenyl)-3-oxo-2,3-dihydropyridazine-4-carboxamide hydrogen chloride: Prepared from (R)—N-(4-(3-((2,2-dimethyl-1,3-dioxolan-4-yl)methoxy)-1-(4-methoxybenzyl)-1H-pyrazolo[3,4-b]pyridin-4-yloxy)-3-fluorophenyl)-2-(4-fluorophenyl)-3-oxo-2,3-dihydropyridazine-4-carboxamide according to the procedure of Example 1, Step L. Purified by silica gel flash column chromatography (SiO2, 90:9.9:0.1=dichloromethane:MeOH: aqueous 30% NH₄OH) to provide the product as a solid (29 mg, 65%). LRMS (ESI pos) m/e 551.0 (M+1). ¹H NMR (400 MHz, DMSO) δ 12.64 (s, 1H), 11.69 (s, 1H), 8.38 (d, 1H), 8.26 (m, 2H), 8.03 (dd, 1H), 7.69 (m, 2H), 7.58 (d, 1H), 7.47 (t, 1H), 7.41 (t, 2H), 6.19 (d, 1H), 4.93 (d, 1H), 4.65 (t, 1H), 4.34 (dd, 1H), 4.21 (dd, 1H), 3.86 (m, 1H), 3.44 (m, 2H); ¹⁹F NMR (376 MHz, DMSO) δ−112.9, −128.2.

### Example 13

### N-(3-fluoro-4-(3-(1-methylpiperidin-4-yloxy)-1H-pyrazolo[3,4-b]pyridin-4-yloxy)phenyl)-4-(4-fluorophenyl)-3-oxo-3,4-dihydropyridazine-2-carboxamide dihydrochloride

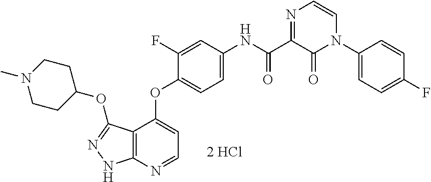

Step A: Preparation of 5-chloro-1-(4-fluorophenyl)-3-methoxypyridin-2(1H)-one: 3,5-Dichloro-1-(4-fluorophenyl)pyrazin-2(1H)-one (13.0 g, 50.2 mmol; prepared by according to the general methods described by M. Tutonda, et al., Tetrahedron, 1990, 46, 5715) dissolved in absolute methanol (100 mL) was treated with sodium methoxide (6.78 g, 125 mmol). The reaction mixture was stirred at room temperature for 1 hour. The mixture was then neutralized with 2 N HCl (Et₂O solution), and the solvent was evaporated under reduced pressure. The residue was treated with EtOAc, washed with 0.5 N HCl, dried over MgSO₄, and concentrated under reduced pressure to give the desired product (12.8 g, 100%). LRMS (ESI pos) m/e 254.9, 256.9 (M+1, Cl pattern).

Step B: Preparation of 1-(4-fluorophenyl)-3-methoxypyridin-2(1H)-one: K₂CO₃ (1.09 g, 7.85 mmol) and 10% Pd/C (0.42 g, 0.39 mmol) were added to 5-chloro-1-(4-fluorophenyl)-3-methoxypyridin-2(1H)-one (2.0 g, 7.85 mmol) in MeOH (100 mL) at room temperature under a H₂ atmosphere. The reaction was stirred for 6 hours. The reaction mixture was filtered with MeOH and concentrated under reduced pressure. The crude was treated with CH₂Cl₂, washed with water, dried over MgSO₄, and concentrated to give the desired product (1.55 g, 90%). LRMS (ESI pos) m/e 221.0 (M+1).

Step C: Preparation of 3-chloro-1-(4-fluorophenyl)pyrazin-2(1H)-one: POCl₃ (5.6 mL, 61.3 mmol) was added drop wise to a solution of 1-(4-fluorophenyl)-3-methoxypyridin-2(1H)-one in DMF (30 mL) with stirring at 0° C. This was followed by heating at 90° C. for 1.5 hours. The residue was cooled to 0° C., quenched by adding saturated sodium acetate solution, extracted with CH₂Cl₂, washed with water, dried over MgSO₄, and concentrated. The crude was purified by silica gel flash column chromatography (0.7% MeOH in CH₂Cl₂) to afford the desired product (3.52 g, 64%). LRMS (ESI pos) m/e 224.9, 227.0 (M+1, Cl pattern).

Step D: Preparation of 4-(4-fluorophenyl)-3-oxo-3,4-dihydropyridazine-2-carbonitrile: A mixture of 3-chloro-1-(4-fluorophenyl)pyrazin-2(1H)-one (3.52 g, 15.7 mmol), CuCN (2.81 g, 31.3 mmol) and N-methylpyrrolidone (30 mL) was heated for 5.5 hours at 150° C. while being stirred. The residue was triturated with hot CHCl₃ and filtered over charcoal. The filtrate was evaporated and concentrated under reduced pressure. The residue was triturated with CH₂Cl₂, and the solution was concentrated. The crude was purified by silica gel flash column chromatography (3:1=CH₂Cl₂:hexane then CH₂Cl₂) to afford the desired product (0.78 g, 23%). LRMS (ESI pos) m/e 215.9 (M+1).

Step E: Preparation of 4-(4-fluorophenyl)-3-oxo-3,4-dihydropyridazine-2-carboxylic acid: A mixture of 4-(4-fluorophenyl)-3-oxo-3,4-dihydropyridazine-2-carbonitrile (0.42 g, 1.95 mmol) and H₂SO₄ (4.16 mL, 78.1 mmol) was stirred at ambient temperature for 17 hours. Then the reaction mixture (amide intermediate) was added to MeOH (50 mL), and then the reaction was heated at 70° C. for 2.5 hours. The reaction mixture was quenched with ice-water and treated with aqueous 2N sodium hydroxide solution at 0° C. The mixture was acidified with aqueous 1N HCl, extracted with EtOAc, dried over MgSO₄, and concentrated to afford the desired product (0.315 g, 69% for 3-step process in one pot reaction), which was rinsed with Et₂O.

Step F: Preparation of 3-fluoro-4-(1-(4-methoxybenzyl)-3-(1-methylpiperidin-4-yloxy)-1H-pyrazolo[3,4-b]pyridin-4-yloxy)aniline: Prepared from 4-(1-(4-methoxybenzyl)-3-iodo-1H-pyrazolo[3,4-b]pyridin-4-yloxy)-3-fluorobenzenamine (prepared as in Example 1, Step E) according to the procedure of Example 1, Step I. Purified by silica gel flash column chromatography (SiO₂, 5% MeOH in DCM) to provide the product as a solid (52 mg, 54%). LRMS (APCI pos) m/e 478.3 (M+1).

Step G: Preparation of N-(3-fluoro-4-(1-(4-methoxybenzyl)-3-(1-methylpiperidin-4-yloxy)-1H-pyrazolo[3,4-b]pyridin-4-yloxy)phenyl)-4-(4-fluorophenyl)-3-oxo-3,4-dihydropyridazine-2-carboxamide: Prepared from 3-fluoro-4-(1-(4-methoxybenzyl)-3-(1-methylpiperidin-4-yloxy)-1H-pyrazolo[3,4-b]pyridin-4-yloxy)aniline and 4-(4-fluorophenyl)-3-oxo-3,4-dihydropyridazine-2-carboxylic acid according to the procedure of Example 1, Step J. Purified by silica gel flash column chromatography (SiO₂, 5 to 10% MeOH in DCM) to provide the product as a solid (13 mg, 42%). LRMS (ESI pos) m/e 694.2 (M+1).

Step H: Preparation of N-(3-fluoro-4-(3-(1-methylpiperidin-4-yloxy)-1H-pyrazolo[3,4-b]pyridin-4-yloxy)phenyl)-4-(4-fluorophenyl)-3-oxo-3,4-dihydropyridazine-2-carboxamide dihydrochloride: Prepared from N-(3-fluoro-4-(1-(4-methoxybenzyl)-3-(1-methylpiperidin-4-yloxy)-1H-pyrazolo[3,4-b]pyridin-4-yloxy)phenyl)-4-(4-fluorophenyl)-3-oxo-3,4-dihydropyridazine-2-carboxamide according to the procedure of Example 1, Step L. Purified by silica gel flash column chromatography (SiO₂, 9:0.5:0.5=dichloromethane:MeOH: aqueous 30% NH₄OH) to provide the product as a solid (3 mg, 28%). LRMS (ESI pos) m/e 574.1 (M+1). ¹14 NMR (400 MHz, CD₃OD) δ 8.20 (d, 1H), 8.0 (dd, 1H), 7.88 (d, 1H), 7.75 (d, 1H), 7.57 (m, 2H), 7.45 (d, 1H), 7.30 (m, 3H), 6.27 (d, 1H), 4.88 (m, 1H), 2.75 (m, 2H), 2.42 (m, 2H), 2.29 (s, 3H), 2.07 (m, 2H), 1.93 (m,2H); ¹⁹F NMR (376 MHz, CD₃OD) δ−113.5, −130.0.

Example 14

### N-(4-(3-((1-ethylpiperidin-4-yl)methoxy)-1H-pyrazolo[3,4-b]pyridin-4-yloxy)-3-fluorophenyl)-4-(4-fluorophenyl)-3-oxo-3,4-dihydropyridazine-2-carboxamide dihydrochloride

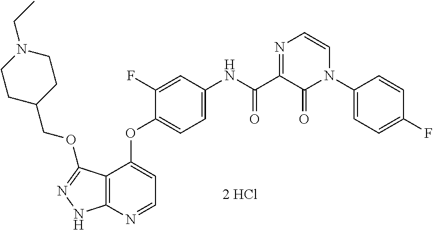

Step A: Preparation of N-(3-fluoro-4-(1-(4-methoxybenzyl)-3-(piperidin-4-ylmethoxy)-1H-pyrazolo[3,4-b]pyridin-4-yloxy)phenyl)-4-(4-fluorophenyl)-3-oxo-3,4-dihydropyridazine-2-carboxamide: Prepared by a 2-step process from tert-butyl 4-((4-(4-amino-2-fluorophenoxy)-1-(4-methoxybenzyl)-1H-pyrazolo[3,4-b]pyridin-3-yloxy)methyl)piperidine-1-carboxylate (prepared as in Example 7, Step A), 4-(4-fluorophenyl)-3-oxo-3,4-dihydropyridazine-2-carboxylic acid (prepared as in Example 13, Step E), and TFA according to the procedure of Example 1, Steps J and K. Purified by silica gel flash column chromatography (SiO₂, 92:6.9:0.1=dichloromethane:MeOH: aqueous 30% NH₄OH) to afford the desired product (0.165 g, 67% yield for 2 step process). LRMS (ESI pos) m/e 694.1 (M+1).

Step B: Preparation of N-(4-(3-((1-ethylpiperidin-4-yl)methoxy)-1H-pyrazolo[3,4-b]pyridin-4-yloxy)-3-fluorophenyl)-4-(4-fluorophenyl)-3-oxo-3,4-dihydropyridazine-2-carboxamide dihydrochloride: Prepared by a 2-step process from N-(3-fluoro-4-(1-(4-methoxybenzyl)-3-(piperidin-4-ylmethoxy)-1H-pyrazolo[3,4-b]pyridin-4-yloxy)phenyl)-4-(4-fluorophenyl)-3-oxo-3,4-dihydropyridazine-2-carboxamide according to the procedure of Example 2, Steps A and B. Purified by silica gel flash column chromatography (SiO₂, 92:7.9:0.1=dichloromethane:MeOH: aqueous 30% NH₄OH) to afford the desired product (29 mg, 37% yield for 2 step process). LRMS (ESI pos) m/e 602.1 (M+1). ¹H NMR (400 MHz, CD₃OD) δ 8.23 (d, 1H), 8.03 (dd, 1H), 7.91 (d, 1H), 7.79 (d, 1H), 7.60 (m, 2H), 7.48 (d, 1H), 7.33 (m, 3H), 6.31 (d, 1H), 4.21 (d, 2H), 3.02 (m, 3H), 2.44 (q, 2H), 2.05 (t, 2H), 1.86 (m, 3H), 1.45 (m, 2H), 1.09 (t, 3H); ¹⁹F NMR (376 MHz, CD₃OD) δ−113.5, −130.0.

Example 15

### 1-(difluoromethyl)-N-(3-fluoro-4-(3-(1-methylpiperidin-4-yloxy)-1H-pyrazolo[3,4-b]piperidin-4-yloxy)phenyl)-2-oxo-1,2-dihydropyridine-3-carboxamide dihydrochloride

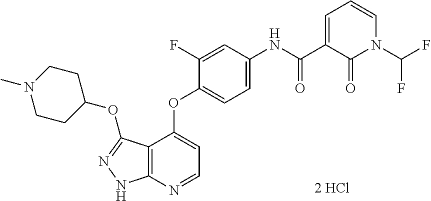

Step A: Preparation of 1-(difluoromethyl)-2-oxo-1,2-dihydropyridine-3-carboxylic acid: Lithium hydride (71 mg, 9.0 mmol) was added to a solution of 2-oxo-1,2-dihydropyridine-3-carboxylic acid (0.50 g, 3.60 mmol) in NMP (10 mL) at 0° C. After stirring for 15 minutes, lithium bromide (0.78 g, 9.0 mmol) and sodium 2-chloro-2,2-difluoroacetate (1.1 g, 9.0 mmol) were successively added. The reaction mixture was heated at 70° C. for 17 hours. The reaction was cooled to 0° C. and quenched with aqueous 1.0 N HCl. The precipitate was filtered with aqueous 1 N HCl, and then the filtrate was extracted with EtOAc. The organic phase was washed with brine, dried over MgSO₄, and concentrate under reduced pressure to give the crude material. The crude material was rinsed with Et₂O to afford the desired product (0.42 g, 62%).

Step B: Preparation of 1-(difluoromethyl)-N-(3-fluoro-4-(1-(4-methoxybenzyl)-3-(1-methylpiperidin-4-yloxy)-1H-pyrazolo[3,4-b]pyridin-4-yloxy)phenyl)-2-oxo-1,2-dihydropyridine-3-carboxamide: Prepared from 3-fluoro-4-(1-(4-methoxybenzyl)-3-(1-methylpiperidin-4-yloxy)-1H-pyrazolo[3,4-b]pyridin-4-yloxy)aniline (prepared as in Example 13, Step F) and 1-(difluoromethyl)-2-oxo-1,2-dihydropyridine-3-carboxylic acid according to the procedure of Example 13, Step G. Purified by silica gel flash column chromatography (SiO₂, 5 to 95:4.9:0.1=dichloromethane:MeOH: aqueous 30% NH₄OH) to provide the product as a solid (18.5 mg, 80%). LRMS (ESI pos) m/e 649.1 (M+1).

Step C: Preparation of 1-(difluoromethyl)-N-(3-fluoro-4-(3-(1-methylpiperidin-4-yloxy)-1H-pyrazolo[3,4-b]pyridin-4-yloxy)phenyl)-2-oxo-1,2-dihydropyridine-3-carboxamide dihydrochloride: Prepared from 1-(difluoromethyl)-N-(3-fluoro-4-(1-(4-methoxybenzyl)-3-(1-methylpiperidin-4-yloxy)-1H-pyrazolo[3,4-b]pyridin-4-yloxy)phenyl)-2-oxo-1,2-dihydropyridine-3-carboxamide according to the procedure of Example 1, Step L. Purified by silica gel flash column chromatography (SiO2, 93:6.9:0.1=dichloromethane:MeOH: aqueous 30% NH₄OH) to provide the product as a solid (6.8 mg, 55%). LRMS (ESI pos) m/e 529.0 (M+1). ¹H NMR (400 MHz, CD₃OD/CDCl₃) δ 8.70 (dd, 1H), 8.21 (d, 1H), 7.98 (td, 1H), 7.88 (t, 1H), 7.43 (m, 1H), 7.28 (t, 1H), 6.77 (t, 1H), 6.26 (d, 1H), 4.94 (m, 1H), 2.76 (m, 2H), 2.43 (m, 2H), 2.31 (s, 3H), 2.14 (m, 2H), 2.01 (m,2H); ¹⁹F NMR (376 MHz, CD₃OD/CDCl₃) δ−104.9, −128.1.

Example 16

### (S)—N-(3-fluoro-4-(3-(1-methylpiperidin-3-yloxy)-1H-pyrazolo[3,4-b]pyridin-4-yloxy)phenyl)-2-(4-fluorophenyl)-3-oxo-2,3-dihydropyridazine-4-carboxamide

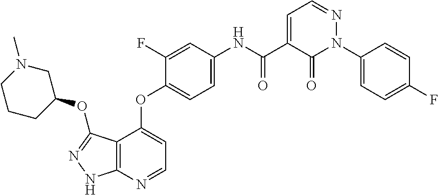

Step A: Preparation of (S)-tert-butyl 3-(4-(4-amino-2-fluorophenoxy)-1-(4-methoxybenzyl)-1H-pyrazolo[3,4-b]pyridin-3-yloxy)piperidine-1-carboxylate: Prepared according to the procedure described in Example 1, Step I. 4-(1-(4-Methoxybenzyl)-3-iodo-1H-pyrazolo[3,4-b]pyridin-4-yloxy)-3-fluorobenzenamine (0.30 g, 0.612 mmol), (S)-tert-butyl 3-hydroxypiperidine-1-carboxylate (3.69 g, 18.4 mmol), 1,10-phenanthroline (0.110 g, 0.612 mmol), CuI (0.116 g, 0.612 mmol), and KF on Al₂O₃ (0.444 g, 3.06 mmol, 40% Wt) were used to give the crude product. The crude product was purified by preparative TLC (3×1 mm) eluting with 3% MeOH/DCM to give the title compound (60 mg, 17%). LRMS (APCI pos) m/e 564.3 (M+1).

Step B: Preparation of (S)-tert-butyl 3-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidine-1-carboxylate: Prepared according to the procedure described in Example 1, Step J. 2-(4-Fluorophenyl)-3-oxo-2,3-dihydropyridazine-4-carboxylic acid (54 mg, 0.234 mmol), EDCI (122 mg, 0.64 mmol), and HOBt-H₂O (98 mg, 0.64 mmol), (S)-tert-butyl 3-(4-(4-amino-2-fluorophenoxy)-1-(4-methoxybenzyl)-1H-pyrazolo[3,4-b]pyridin-3-yloxy)piperidine-1-carboxylate (0.60 g, 0.106 mmol), Et₃N (0.089 mL, 0.64 mmol) were used to give the crude product. The crude product was purified by chromatography (5 g Isolute) eluting with 1% MeOH/DCM to give the title compound (24 mg, 29%). LRMS (APCI pos) m/e 780.7 (M+1).

Step C: Preparation of (S)—N-(3-fluoro-4-(1-(4-methoxybenzyl)-3-(piperidin-3-yloxy)-1H-pyrazolo[3,4-b]pyridin-4-yloxy)phenyl)-2-(4-fluorophenyl)-3-oxo-2,3-dihydropyridazine-4-carboxamide: Prepared according to the procedure described in Example 1, Step K. A mixture of (S)-tert-butyl 3-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)piperidine-1-carboxylate (0.024 g, 0.031 mmol) and TFA (2.5 mL, 31.0 mmol) in CH₂Cl₂ (5 mL) was stirred at room temperature for 18 hours. The reaction mixture was concentrated under reduced pressure using toluene to azeotrope, and the crude was partitioned between DCM and 10% Na₂CO₃ (aq.). The organic layer was washed with brine, dried over sodium sulfate and evaporated to give the product as free base (18 mg, 86%). LRMS (APCI pos) m/e 670.7 (M+1).

Step D: Preparation of (S)—N-(3-fluoro-4-(1-(4-methoxybenzyl)-3-(1-methylpiperidin-3-yloxy)-1H-pyrazolo[3,4-b]pyridin-4-yloxy)phenyl)-2-(4-fluorophenyl)-3-oxo-2,3-dihydropyridazine-4-carboxamide: (S)—N-(3-Fluoro-4-(1-(4-methoxybenzyl)-3-(piperidin-3-yloxy)-1H-pyrazolo[3,4-b]pyridin-4-yloxy)phenyl)-2-(4-fluorophenyl)-3-oxo-2,3-dihydropyridazine-4-carboxamide (18 mg, 0.026 mmol) was added to a small round bottom flask and dissolved in THF (2 mL). Formaldehyde (5.9 μL, 0.079 mmol), acetic acid (d1.049; 0.76 μL, 0.013 mmol), and finally NaBH(OAc)₃ (16.84 mg, 0.079 mmol) were added to the flask. The contents were allowed to stir at room temperature for 30 minutes. The reaction mixture was partitioned between water (10 mL) and ethyl acetate (10 mL). The organic layer was washed with brine (10 mL), dried over Na₂SO₄, and evaporated to give the crude product (18 mg, 90% purity, 88%). LRMS (APCI pos) m/e 694.2 (M+1).

Step E: Preparation of (S)—N-(3-fluoro-4-(3-(1-methylpiperidin-3-yloxy)-1H-pyrazolo[3,4-b]pyridin-4-yloxy)phenyl)-2-(4-fluorophenyl)-3-oxo-2,3-dihydropyridazine-4-carboxamide: Prepared according to the procedure described in Example 1, Step L. (S)—N-(3-Fluoro-4-(1-(4-methoxybenzyl)-3-(1-methylpiperidin-3-yloxy)-1H-pyrazolo[3,4-b]pyridin-4-yloxy)phenyl)-2-(4-fluorophenyl)-3-oxo-2,3-dihydropyridazine-4-carboxamide (18 mg, 0.026 mmol) and a large excess of TFA (3-5 mL) were used to give crude material. The crude material was purified by preparative TLC eluting with 90/10/1 DCM/MeOH/NH₄OH. The product was recovered as a free-base (3.8 mg) and converted to the HCl salt (4.8 mg 2 HCl, 29%) according to Example 1, Step L. LRMS (APCI pos) m/e 574.2 (M+1). ¹H-NMR (400 MHz, DMSO-d6) δ 12.65 (s, 1H), 11.68 (s, 1H), 8.39 (d, 1H), 8.25 (dd, 2H), 8.03 (dd, 1H), 7.68 (m, 2H), 7.55 (d, 1H), 7.40 (m, 31.1), 6.25 (d, 1H), 4.78 (m, 1H), 2.98 (d, 2H), 2.15 (s, 3H), 2.08 (m, 2H), 1.77 (m, 2H), 1.4 (m, 2H).

### Example 17

### N-(3-Fluoro-4-(3-(cis-3-hydroxypiperidin-4-hydroxy)-1H-pyrazolo[3,4-b]pyridin-4-yloxy)phenyl)-2-(4-fluorophenyl)-3-oxo-2,3-dihydropyridazine-4-carboxamide

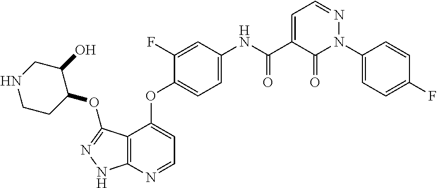

Step A: Preparation of cis-tert-butyl 3,4-dihydroxypiperidine-1-carboxylate: A 250 mL, round-bottomed flask was charged with tert-butyl 5,6-dihydropyridine-1(2H)-carboxylate (5.0 g, 27.29 mmol), osmium(VIII)oxide (13.87 mL, 1.36 mmol), 4-methylmorpholine 4-oxide (5.5 g, 40.93 mmol) and acetone (100 mL). The reaction mixture was stirred at room temperature until LC-MS shows that the starting material had been consumed (72 hours). Then the reaction was partitioned between EtOAc (300 mL) and water (200 mL). The phases were separated, and the aqueous phase was extracted with EtOAc (3×300 mL). The combined organic layers were dried (Na₂SO₄), filtered and concentrated to yield a crude product. The crude product was purified by silica gel chromatography (EtOAc/Hexane from 1/4 to 1/0, v/v) to afford the desired product (5.89 g, 99%). m/e 118 (M−99). ¹H NMR (400 MHz,DMSO-d₆) δ 3.86 (m, 1H), 3.76 (m, 1H), 3.54 (m, 2H), 3.44 (m, 1H), 3.29 (m, 1H), 1.81 (m, 1H), 1.68 (m, 1H), 1.46 (s, 9H).

Step B: Preparation of cis-tert-butyl 4-(4-(4-amino-2-fluorophenoxy)-1-(4-methoxybenzyl)-1H-pyrazolo[3,4-b]pyridin-3-yloxy)-3-hydroxypiperidine-1-carboxylate: A 100 mL, round-bottomed flask was charged with 4-(1-(4-methoxybenzyl)-3-iodo-1H-pyrazolo[3,4-b]pyridin-4-yloxy)-3-fluorobenzenamine (300.0 mg, 0.612 mmol; prepared as in Example 1, Step E), cis-tert-butyl 3,4-dihydroxypiperidine-1-carboxylate (3.99 g, 18.36 mmol), copper(I)iodide (116.5 mg, 0.612 mmol), 1,10-phenanthroline (110.27 mg, 0.612 mmol), potassium fluoride (622.1 mg, 4.28 mmol) and toluene (5 mL). The reaction mixture was stirred at room temperature until LC-MS shows that the starting material has been consumed (48 hours). Then the reaction was partitioned between EtOAc (300 mL) and water (200 mL). The phases were separated, and the aqueous phase was extracted with EtOAc (3×200 mL). The combined organic layers were dried (Na₂SO₄), filtered and concentrated to yield a crude product. The crude product was purified by silica gel chromatography (EtOAc/Hexane from 1/1 to 1/0, v/v) to afford impure product (0.20 g, 56.5%), which contained the other isomer. LRMS (APCI pos): m/e 580.2 (M+1).

Step C: Preparation of cis-tert-butyl 4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)-3-hydroxypiperidine-1-carboxylate: A 100 mL, round-bottomed flask was charged with cis-tert-butyl 4-(4-(4-amino-2-fluorophenoxy)-1-(4-methoxybenzyl)-1H-pyrazolo[3,4-b]pyridin-3-yloxy)-3-hydroxypiperidine-1-carboxylate (0.20 g, 0.346 mmol), 2-(4-fluorophenyl)-3-oxo-2,3-dihydropyridazine-4-carboxylic acid (0.12 g, 0.519 mmol; prepared as in Example 1, Step H), N1-((ethylimino)methylene)-N3,N3-dimethylpropane-1,3-diamine hydrochloride (0.1 g, 0.519 mmol), 1H-benzo[d][1,2,3]triazol-1-ol hydrate (0.08 g, 0.519 mmol), N-ethyl-N-isopropylpropan-2-amine (0.31 ml, 1.73 mmol) and DCM (1.5 mL). The reaction mixture was stirred at room temperature until LC-MS shows that the starting material has been consumed (overnight). Then the reaction was partitioned between EtOAc (100 mL) and water (100 mL). The phases were separated, and the aqueous phase was extracted with EtOAc (3×100 mL). The combined organic layers were dried (Na₂SO₄), filtered and concentrated to yield a crude product. The crude product was purified by silica gel chromatography (DCM/MeOH from 100/1 to 50/1, v/v) to afford impure product (126 mg, 46%), which contained the other isomer. LRMS (APCI pos): m/e 796.6 (M+1).

Step D: Preparation of N-(3-fluoro-4-(3-(cis-3-hydroxypiperidin-4-yloxy)-1H-pyrazolo[3,4-b]pyridin-4-yloxy)phenyl)-2-(4-fluorophenyl)-3-oxo-2,3-dihydropyridazine-4-carboxamide: A 100 mL, round-bottomed flask was charged with cis-tert-butyl 4-(4-(2-fluoro-4-(2-(4-fluorophenyl)-3-oxo-2,3-dihydropyridazine-4-carboxamido)phenoxy)-1-(4-methoxybenzyl)-1H-pyrazolo[3,4-b]pyridin-3-yloxy)-3-hydroxypiperidine-1-carboxylate (126.3 mg, 0.16 mmol) and 2,2,2-trifluoroacetic acid (3.62 g, 31.74 mmol). The reaction mixture was stirred at 60° C. until LC-MS showed that the starting material had been consumed (48 hours). Then the CF₃COOH was removed under reduced pressure. The residue was purified by silica gel chromatography (DCM/7 M NH₃ in MeOH from 100/1 to 10/1, v/v) to afford the desired product (7.2 mg, 7.9%). LRMS (APCI pos): >90% purity, 254 nm, m/e 576.1 (M+1). ¹H NMR (400 MHz, CDCl₃) δ 8.34 (d, 1H), 8.30 (d, 1H), 8.22 (d, 1H), 8.01 (dd, 1H), 7.67 (m, 2H), 7.46 (m, 1H), 7.35 (d, 1H), 7.25-7.36 (m, 2H), 6.25 (d, 1H), 5.09 (m, 1H), 4.17 (m, 1H), 3.12 (m, 2H), 2.93 (m, 1H), 2.76 (m, 1H), 2.19 (m, 1H), 1.91 (m, 1H).

### Example 18

### Preparation of N-(4-(3-(1-ethylpiperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide dihydrochloride

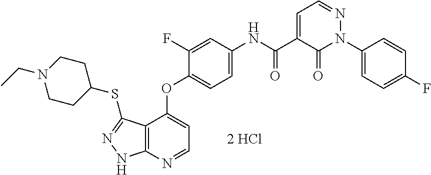

Step A: Preparation of tert-butyl 4-mercaptopiperidine-1-carboxylate: Prepared using methodology described in J. Med. Chem. 1993, 36, 3261. H₂S gas was bubbled through a stirred mixture of tert-butyl 4-oxopiperidine-1-carboxylate (1 g, 5 mmol) in isopropanol (10 mL) for 10 minutes. The mixture was allowed to stir for 18 hours at room temperature. The reaction was then degassed by bubbling N₂ for 10 minutes. NaBH₄ (285 mg, 7.53 mmol) was carefully added to the mixture, and it was heated to 80° C. for 2 hours. After cooling to room temperature, the mixture was concentrated. The crude was partitioned between diethyl ether (20 mL) and water (20 mL). The phases were separated, and the aqueous was re-extracted with diethyl ether (2×10 mL). The combined organic phases were dried (Na₂SO₄), filtered, and concentrated. The crude was purified by Biotage Flash 40, eluting with 10% EtOAc/hexanes (1 L). The product was obtained as a viscous oil (345 mg, 31%). ¹H NMR (400 MHz, CDCl₃) δ 4.00 (m, 2H), 2.94 (m, 1H), 2.87 (m, 2H), 1.95 (m, 2H), 1.49 (m, 2H), 1.46 (s, 9H).

Step B: Preparation of tert-butyl 4-(1-(4-methoxybenzyl)-4-(4-amino-2-fluorophenoxy)-1H-pyrazolo[3,4-b]pyridin-3-ylthio)piperidine-1-carboxylate: CuI (7.6 mg, 0.040 mmol) and 1,10-phenanthroline (11 mg, 0.060 mmol) were added to a stirred mixture of tert-butyl 4-mercaptopiperidine-1-carboxylate (109 mg, 0.50 mmol) and 4-(1-(4-methoxybenzyl)-3-iodo-1H-pyrazolo[3,4-b]pyridin-4-yloxy)-3-fluorobenzenamine (49 mg, 0.100 mmol; prepared as in Example 1, Step E) in toluene (0.2 mL), followed by KF on Al₂O₃ (73 mg, 0.50 mmol). The mixture was stirred at 100° C. for 2 hours. The reaction had gone to >95% conversion. The crude reaction mixture was loaded directly on to a preparative TLC plate (0.5 mm thickness, Rf=0.18) and eluted with 1:1 EtOAc/hexanes. The desired product co-eluted with a des-iodo by-product. The mixture was carried forward without further purification at this step. LRMS (ESI+): m/z 480, 580 (M+1) detected.

Step C: Preparation of tert-butyl 4-(1-(4-methoxybenzyl)-4-(2-fluoro-4-(1-(4-fluorophenyl)-6-oxo-1,6-dihydropyridazine-5-carboxamido)phenoxy)-1H-pyrazolo[3,4-b]pyridin-3-ylthio)piperidine-1-carboxylate: EDCI (40 mg, 0.21 mmol) was added to a stirred mixture of 2-(4-fluorophenyl)-3-oxo-2,3-dihydropyridazine-4-carboxylic acid (24 mg, 0.10 mmol; prepared as in Example 1, Step H), HOBt-hydrate (32 mg, 0.21 mmol), and triethylamine (0.029 ml, 0.21 mmol) in DCM (0.2 mL) at room temperature. The reaction mixture was stirred for 15 minutes at room temperature. tert-Butyl 4-(1-(4-methoxybenzyl)-4-(4-amino-2-fluorophenoxy)-1H-pyrazolo[3,4-b]pyridin-3-ylthio)piperidine-1-carboxylate (20 mg, 0.035 mmol) was then added. The resulting solution was stirred for 18 hours at room temperature. The reaction had only gone to 50% conversion so to a second vial was added the above activated acid reagents: EDCI (40 mg, 0.21 mmol, 2 equivalents), 2-(4-fluorophenyl)-3-oxo-2,3-dihydropyridazine-4-carboxylic acid (24 mg, 0.10 mmol, 2 equivalents), HOBt-hydrate (32 mg, 0.21 mmol, 2 equivalents), triethylamine (0.029 mL, 0.21 mmol, 2 equivalents) and DCM (0.5 mL). The mixture was stirred for 15 minutes, and the original reaction mixture was added. The stirring was continued for 2 hours at room temperature. LCMS indicated complete conversion of the aniline starting material. The entire reaction mixture was loaded on to a preparative TLC plate (0.5 mm thickness, Rf=0.18) and eluted with 1:1 EtOAc/hexanes. The desired product and the des-iodo by-product continued to co-elute. The mixture was carried forward without further purification at this step. LRMS (ESI+): m/z 796 (M+1) detected.

Step D: Preparation of N-(4-(1-(4-methoxybenzyl)-3-(piperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide bis-trifluoroacetic acid salt: A mixture of tert-butyl 4-(1-(4-methoxybenzyl)-4-(2-fluoro-4-(1-(4-fluorophenyl)-6-oxo-1,6-dihydropyridazine-5-carboxamido)phenoxy)-1H-pyrazolo[3,4-b]pyridin-3-ylthio)piperidine-1-carboxylate (28 mg, 0.035 mmol) and trifluoroacetic acid (1 mL) was stirred for 5 minutes at room temperature. The mixture was then concentrated in vacuo using toluene to azeotrope (3×5 mL). The mixture was carried forward in reductive amination without workup or purification. LRMS (ESI+): m/z 696 (M+1) detected.

Step E: Preparation of N-(4-(1-(4-methoxybenzyl)-3-(1-ethylpiperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide: Sodium triacetoxyborohydride (11 mg, 0.054 mmol) was added to a stirred mixture of N-(4-(1-(4-methoxybenzyl)-3-(piperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide bis-trifluoroacetic acid salt (33 mg, 0.0357 mmol), acetaldehyde (2 mg, 0.05 mmol), in DCM (0.5 mL). The reaction mixture was stirred at room temperature for 2 hours. The reaction had gone to conversion by LCMS. Water (5 mL) was added, and the aqueous layer was extracted with DCM (3×5 mL). The organic layers were combined and dried (Na₂SO₄). The product was concentrated and purified by preparative TLC (0.5 mm thickness, Rf=0.30), eluting with 10% MeOH in CHCl₃. The product was obtained as a waxy solid (22 mg, 85%). LRMS (ESI+): m/z 724 (M+1) detected.

Step F: Preparation of N-(4-(3-(1-ethylpiperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide dihydrochloride: A mixture of N-(4-(1-(4-methoxybenzyl)-3-(1-ethylpiperidin-4-ylthio)-1H-pyrazolo[3,4-b]pyridin-4-yloxy)-3-fluorophenyl)-2-(4-fluorophenyl)-3-oxo-2,3-dihydropyridazine-4-carboxamide (22 mg, 0.030 mmol) in trifluoroacetic acid (0.2 mL) was heated to 80° C. for 18 hours and then concentrated in vacuo using toluene to azeotrope (3×5 mL). The residue was partitioned between EtOAc (5 mL) and saturated NaHCO₃ (aq., 5 mL). The phases were separated, and the aqueous phase was re-extracted with EtOAc (3×5 mL). The combined organic phases were dried (Na₂SO₄), filtered, and concentrated. The crude was purified by preparative TLC, eluting with 20% MeOH/DCM. The product was acidified with 2N HCl in ether (0.5 mL) and concentrated using EtOH to azeotrope (3×5 mL). The product was obtained as a solid (4 mg, 19%). LRMS (ESI+): m/z 604 (M+1) detected. ¹H NMR (400 MHz, CDCl₃, free base) δ 11.88 (m, 1H), 8.44 (m, 1H), 8.36 (m, 1H), 8.28 (m, 1H), 8.00 (m, 1H), 7.63 (m, 2H), 7.43 (m, 1H), 7.26 (m, 3H), 6.29 (m, 1H), 3.78 (m, 1H), 3.08 (m, 2H), 2.64 (m, 2H), 2.32 (m, 3H), 1.97 (m, 2H), 1.22 (m, 3H).

### Example 19

### Preparation of 2-(4-(3-((1-ethylpiperidin-4-yl)methoxy)-1H-pyrazolo[3,4-b]pyridin-4-yloxy)-3-fluorophenylamino)-N-(4-fluorophenyl)nicotinamide

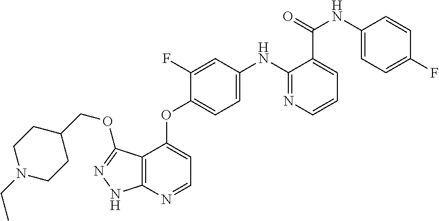

Step A: Preparation of 2-amino-N-(4-fluorophenyl)nicotinamide: A round-bottomed flask was charged with HOBT-H₂O (20.37 g, 133.0 mmol), EDCI (25.50 g, 133.0 mmol), 2-aminonicotinic acid (12.25 g, 88.69 mmol), and DMF (750 mL). After stirring for 30 minutes, Hunig's Base (30.90 mL, 177.4 mmol) was added, followed by 4-fluorobenzenamine (10.65 mL, 110.9 mmol). After stirring for 18 hours, the reaction was poured into water (2 L) and a precipitate formed. After 30 minutes, the precipitate was filtered and dried. LRMS M+1 (231.9) observed.

Step B: Preparation of 2-(3-fluoro-4-methoxyphenylamino)-N-(4-fluorophenyl)nicotinamide: A round bottom flask was charged with cesium carbonate (11.1 g, 34.1 mmol), 4-bromo-2-fluoro-1-methoxybenzene (5.00 g, 24.4 mmol), 2-amino-N-(4-fluorophenyl)nicotinamide (7.61 g, 32.9 mmol) and dioxane (250 mL). After degassing with nitrogen for 10 minutes, Xanphos (0.564 g, 0.975 mmol) and Pd₂dba₃ (0.670 g, 0.732 mmol) were added. The reaction was heated to 90° C. for 48 hours. The reaction was then cooled to room temperature, diluted with water (300 mL), and extracted with EtOAc (300 mL). The organics were separated, dried over sodium sulfate, filtered and concentrated. Purification by silica gel chromatography eluting with dichloromethane/MeOH (3%) followed by pooling and concentration of product containing fractions gave the product (8.50 g, 93%). LRMS M+1 (365.0) observed.

Step C: Preparation of 2-(3-fluoro-4-hydroxyphenylamino)-N-(4-fluorophenyl)nicotinamide: A round-bottomed flask was charged with 2-(3-fluoro-4-methoxyphenylamino)-N-(4-fluorophenyl)nicotinamide (8.00 g, 22.5 mmol) and dichloromethane (75 mL). After cooling to 0° C., BBr₃ (10.9 mL, 115 mmol) was added dropwise over 5 minutes. After addition of BBr₃, a precipitate formed. After 2 hours, the reaction mixture was slowly quenched by pipeting into a flask containing stirring saturated NaHCO₃ (20 mL) and water (150 mL). The mixture was extracted with EtOAc (2×300 mL). The organics were separated, dried over sodium sulfate, filtered and concentrated. The product was isolated (6.25 g, 73%). LRMS M−1 (339.9) observed.

Step D: Preparation of 4-chloro-1-(4-methoxybenzyl)-1H-pyrazolo[3,4-b]pyridine: 1-(4-Methoxybenzyl)-1H-pyrazolo[3,4-b]pyridin-4-ol (3.00 g, 11.75 mmol; prepared according to the procedure of Example 1, Step B) was added as a solid all-at-once to a solution of phosphoryl trichloride (3.227 mL, 35.26 mmol) in dichloroethane (60 mL). The reaction was stirred under N₂ at reflux for 4 hours. The reaction mixture was cooled to room temperature and then poured slowly onto ice water. Saturated NaHCO₃ was slowly added until the reaction mixture was neutral by pH paper and then was extracted with CH₂Cl₂ (added a small amount of methanol to help resolve layers). The aqueous phase was re-extracted with CH₂Cl₂ (2×). The combined organic layers were washed with brine, dried over Na₂SO₄, filtered and concentrated to yield the crude product as an oil. The crude product was purified by silica gel chromatography, eluting with 10:1 hexanes/EtOAc. The desired product (1.056 g, 47%) was obtained as a crystalline solid. ¹H NMR (400 MHz, CDCl₃) δ 8.44 (d, J=5.1 Hz, 1H), 8.09 (s, 1H), 7.32 (m, 2H), 7.13 (d, J=5.1 Hz, 1H), 6.83 (m, 2H), 5.64 (s, 2H), 3.76 (s, 3H). LRMS (APCI pos) m/e 274, 276 (M+, Cl pattern).

Step E: Preparation of 4-chloro-1H-pyrazolo[3,4-b]pyridine: 4-Chloro-1-(4-methoxybenzyl)-1H-pyrazolo[3,4-b]pyridine (1.51 g, 5.50 mmol) was dissolved in neat TFA (8.48 mL, 110 mmol), and the reaction mixture was stirred at 75° C. for 2 hours. The reaction mixture was then concentrated to an oil, and MeOH was added to give a thick precipitate that was filtered and washed with MeOH. The filtrate, which contained the desired product, was concentrated to an oil that was dried in vacuo overnight to yield a waxy solid. The crude solid was partitioned between EtOAc and saturated NaHCO₃. The phases were separated, and the aqueous layer was re-extracted with EtOAc (1×). The combined organic phases were dried over Na₂SO₄, filtered and concentrated to yield the desired product (0.845 g, 100%) as a solid. ¹H NMR (400 MHz, CDCl₃) δ 11.93 (br s, 1H), 8.50 (d, J=5.1 Hz, 1H), 8.20 (s, 1H), 7.21 (m, 1H). LRMS (APCI pos) m/e 154, 156 (M+, Cl pattern).

Step F: Preparation of 4-chloro-3-iodo-1H-pyrazolo[3,4-b]pyridine: Potassium hydroxide flakes (0.931 g, 16.6 mmol) was added to a solution of 4-chloro-1H-pyrazolo[3,4-b]pyridine (0.849 g, 5.53 mmol) in DMF (25 mL), followed by 1₂ (2.53 g, 9.95 mmol). The reaction mixture was stirred at 50° C. for 1.5 hours. The reaction mixture was cooled to room temperature and then quenched with 10% aqueous sodium bisulfite solution until the dark color disappeared, which yielded a precipitate. The resulting suspension was diluted with H₂O, filtered and washed with H₂O to yield a solid. The solid was dissolved with CH₂Cl₂/MeOH, concentrated, and dried in vacuo overnight to yield the desired product (1.41 g, 91%) as a solid. ¹H NMR (400 MHz, DMSO-d₆) δ 8.49 (d, J=5.1 Hz, 1H), 7.35 (d, J=5.1 Hz, 1H). LRMS (APCI pos) m/e 280, 282 (M+, CI pattern).

Step G: Preparation of 4-chloro-3-iodo-1-(4-methoxybenzyl)-1H-pyrazolo[3,4-b]pyridine: K₂CO₃ (1.30 g, 9.38 mmol) and 1-(chloromethyl)-4-methoxybenzene (0.766 mL, 5.63 mmol) was added to a solution of 4-chloro-3-iodo-1H-pyrazolo[3,4-b]pyridine (1.31 g, 4.688 mmol) in DMF (40 mL). The reaction mixture was stirred at room temperature overnight to yield two regioisomeric products is a 5.5:1 ratio by LC-MS. The mixture was partitioned between EtOAc and H₂O. The phases were separated, and the organic layer was washed with brine, dried over Na₂SO₄, filtered and concentrated to afford a solid. The crude product was purified by silica gel chromatography, eluting with 3:1 hexanes/EtOAc and loaded with 10:1:1 CH₂Cl₂/MeOH/THF due to poor solubility. The desired N1-regioisomeric product (1.256 g, 67%) was obtained as a crystalline solid. ¹H NMR (400 MHz, DMSO-d₆) δ 8.51 (d, J=5.1 Hz, 1H), 7.36 (d, J=5.0 Hz, 1H), 7.18 (d, J=8.9 Hz, 2H), 6.83 (d, J=8.4 Hz, 2H), 5.55 (s, 2H), 3.66 (s, 3H). LRMS (APCI pos) m/e 400, 402 (M+, Cl pattern). The undesired N2-regioisomer, 4-chloro-3-iodo-2-(4-methoxybenzyl)-2H-pyrazolo[3,4-b]pyridine, was not isolated.

Step H: Preparation of 2-(4-(1-(4-methoxybenzyl)-3-iodo-1H-pyrazolo[3,4-b]pyridin-4-yloxy)-3-fluorophenylamino)-N-(4-fluorophenyl)nicotinamide: A 100 mL sealable tube was charged with 1-(4-methoxybenzyl)-4-chloro-3-iodo-1H-pyrazolo[3,4-b]pyridine (0.250 g, 0.626 mmol), 2-(3-fluoro-4-hydroxyphenylamino)-N-(4-fluorophenyl)nicotinamide (0.427 g, 1.25 mmol), cesium carbonate (0.408 g, 1.25 mmol), and 1-bromobenzene (6.26 mL, 0.626 mmol) and heated to 160° C. for 18 hours. After cooling to room temperature, the reaction mixture was concentrated to give a solid. The solid was dissolved with EtOAc (100 mL) and washed with brine (2×100 mL). The crude material was purified by silica gel chromatography eluting with 4:1 hexane/EtOAc. Product containing fractions were pooled, and the product isolated (0.35 g, 71%). LRMS M−1 (704.9) observed.

Step I: Preparation of tert-butyl 4-((4-(2-fluoro-4-(3-(4-fluorophenylcarbamoyl)pyridin-2-ylamino)phenoxy)-1-(4-methoxybenzyl)-1H-pyrazolo[3,4-b]pyridin-3-yloxy)methyl)piperidine-1-carboxylate: A sealable tube was charged with 2-(4-(1-(4-methoxybenzyl)-3-iodo-1H-pyrazolo[3,4-b]pyridin-4-yloxy)-3-fluorophenylamino)-N-(4-fluorophenyl)nicotinamide (0.50 g, 0.71 mmol), tert-butyl 4-(hydroxymethyl)piperidine-1-carboxylate (4.58 g, 21.3 mmol), CuI (0.054 g, 0.28 mmol), 1,10-phenanthroline (0.051 g, 0.28 mmol) and toluene (15 mL). KF on Al₂O₃ (0.52 g, 3.5 mmol) was then added, and the reaction mixture was heated to 110° C. for 3 days. Additional CuI (0.054 g, 0.28 mmol) and 1,10-phenanthroline (0.051 g, 0.28 mmol) was added at this point. The reaction was allowed to stir for another 24 hours and was then cooled to room temperature. The reaction mixture was diluted with EtOAc and washed with brine. The organics were dried with sodium sulfate, filtered and concentrated to a crude product that was purified by silica gel chromatography eluting with EtOAc/Hexane 4:1. The product, 425 mgs, 25% pure (contained excess alcohol) (25% yield) was isolated. LRMS M+1 (579.0) observed (fragment).

Step J: Preparation of 2-(3-fluoro-4-(1-(4-methoxybenzyl)-3-(piperidin-4-ylmethoxy)-1H-pyrazolo[3,4-b]pyridin-4-yloxy)phenylamino)-N-(4-fluorophenyl)nicotinamide: A flask was charged with tert-butyl 4-((4-(2-fluoro-4-(3-(4-fluorophenylcarbamoyl)pyridin-2-ylamino)phenoxy)-1-(4-methoxybenzyl)-1H-pyrazolo[3,4-b]pyridin-3-yloxy)methyl)piperidine-1-carboxylate (425 mg, 0.134 mmol) and DCM (10 mL). HCl (5 mL, 2N in Et₂O) was then added. After 72 hours, the reaction was neutralized with Na₂CO₃ (20 mL) and extracted with EtOAc. The organics were dried over sodium sulfate, filtered and concentrated to give a crude product that was loaded onto a small silica gel column with DCM. The impurities were eluted first with DCM. Product was then eluted with DCM/MeOH: NH₃ 7N (97:3). Product containing fractions were pooled and concentrated to an oil (50 mg, 97% pure, 52% yield), which solidified under high vacuum. LRMS M+1 (692.2) observed.

Step K: Preparation of 2-(4-(3-((1-ethylpiperidin-4-yl)methoxy)-1-(4-methoxybenzyl)-1H-pyrazolo[3,4-b]pyridin-4-yloxy)-3-fluorophenylamino)-N-(4-fluorophenyl)nicotinamide: A reaction vial was charged with 2-(3-fluoro-4-(1-(4-methoxybenzyl)-3-(piperidin-4-ylmethoxy)-1H-pyrazolo[3,4-b]pyridin-4-yloxy)phenylamino)-N-(4-fluorophenyl)nicotinamide (50.0 mg, 0.0723 mmol), acetaldehyde (6.37 mg, 0.145 mmol) in DCM (3 mL), and lastly sodium triacetoxy borohydride (30.6 mg, 0.145 mmol). The reaction mixture was stirred at room temperature for 1 hour. The reaction mixture was then treated with water (5 mL), extracted with DCM (3×10 mL), dried over sodium sulfate, and concentrated to, give the crude product, which was purified by preparative TLC (20 cm×20 cm plate, 1.0 mm thickness) eluting with 5% MeOH in DCM. The product was isolated (28 mg, 50% pure, 27% yield) at Rf 0.5 as a mixture of desired and bis reductive amination product. LRMS M+1 (720.2) observed.

Step L: Preparation of 2-(4-(3-((1-ethylpiperidin-4-yl)methoxy)-1H-pyrazolo[3,4-b]pyridin-4-yloxy)-3-fluorophenylamino)-N-(4-fluorophenyl)nicotinamide: A 10 mL sealable vial was charged with 2-(4-(3-((1-ethylpiperidin-4-yl)methoxy)-1-(4-methoxybenzyl)-1H-pyrazolo[3,4-b]pyridin-4-yloxy)-3-fluorophenylamino)-N-(4-fluorophenyl)nicotinamide (44 mg, 0.031 mmol) and TFA (3 mL) and was heated to 75° C. for 18 hours. The reaction was then cooled to room temperature and neutralized by pouring into saturated Na₂CO₃ (20 mL). The mixture was extracted with EtOAc, dried over sodium sulfate, filtered and concentrated. The crude product was purified by C-18 reverse phase silica gel chromatography eluting with dichloromethane. Product containing fractions were pooled and concentrated. The product was suspended in chloroform and made into an HCl salt by adding 3 drops of 2N HCl (in Et₂O). After concentration, the product was isolated as a solid (8.5 mg, 85% pure 39% yield). LRMS M+1 (600.1) observed. ¹H NMR (400 MHz , CDCl₃) δ 10.53 (s, 1H), 8.42 (s, 1H), 8.23 (s, 1H), 8.04 (m, 2H), 7.91 (m, 1H), 7.55 (m, 2H), 7.29 (m, 211), 7.13 (m, 3H), 6.84 (m, 1H), 6.25 (m, 1H), 4.25 (m, 2H), 2.99 (m, 2H), 2.44 (m, 2H), 1.98 (m, 7H), 0.88 (t, 3H). ¹⁹F NMR (376 MHz, CDCl₃) δ−116.7 (1F), −127.7 (1F).

The following compounds in Table 1 were prepared following the above procedures.

TABLE 1

| Ex | | | |
|---|---|---|---|
| # | Structure | Name | NMR |
| 20 | 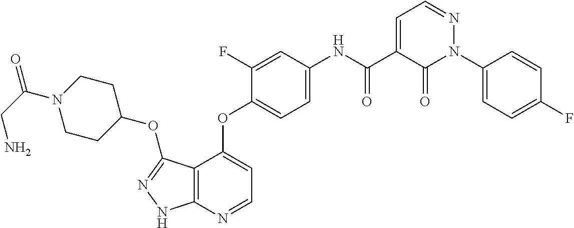 | N-(4-(3-(1-(2- aminoacetyl) piperidin- 4-yloxy)-1H- pyrazolo[3,4-b] pyridin- 4-yloxy)-3- fluorophenyl)-2- (4-fluorophenyl)- 3-oxo-2,3- dihydropyridazine- 4-carboxamide | ¹H NMR (400 MHz, CDCl₃) δ 11.82 (s, 1 H), 8.41 (d, 1 H), 8.29 (d, 1 H), 8.24 (d, 1 H), 7.95 (dd, 1 H), 7.61 (m, 2 H), 7.38 (m, 1 H), 7.25 (m, 3 H), 6.27 (d, 1 H), 5.14 (m, 1 H), 3.82 (m, 1 H), 3.65 (m, 2 H), 3.35 (m, 1 H), 2.17 (s, 2 H), 1.99 (m, 4 H) |
| 21 | 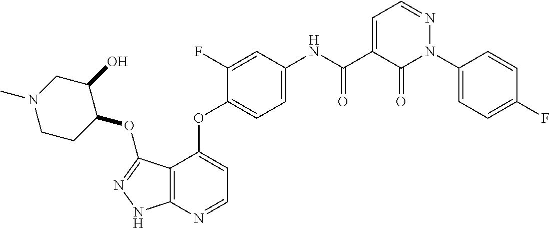 | N-(3-fluoro-4-(3- ((3R,4S)-3-hydroxy- 1-methylpiperidin- 4-yloxy)-1H- pyrazolo[3,4-b] pyridin-4-yloxy) phenyl)-2-(4- fluorophenyl)-3- oxo-2,3-dihydro- pyridazine- 4-carboxamide | ¹H NMR (400 MHz, CDCl₃) d 11.82 (s, 1 H), 10.16 (s, 1 H), 8.41 (d, 1 H), 8.28 (d, 1 H), 8.24 (d, 1 H), 7.96 (dd, 1 H), 7.56-7.63 (m, 2 H), 7.39 (m, 1 H), 7.21-7.29 (m, 4 H), 6.25 (d, 1 H), 4.93 (m, 1 H), 4.15 (m, 1 H), 2.48-2.76 (m, 3 H), 2.32 (s, 3 H), 2.01 (m, 1 H), 1.55-1.78 (m, 2 H) |
| 22 | 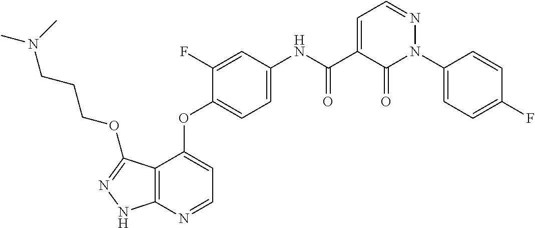 | N-(4-(3-(3- (dimethylamino) propoxy)- 1H-pyrazolo[3,4- b]pyridin-4-yloxy)- 3-fluorophenyl)- 2-(4-fluoro- phenyl)-3-oxo-2,3- dihydropyridazine- 4-carboxamide | ¹H NMR (400 MHz, CDCl₃) δ 11.82 (s, 1 H), 10.26 (s, 1 H), 8.41 (d, J = 4.3 Hz, 1 H), 8.27 (d, J = 5.5 Hz, 1 H), 8.24 (d, J = 4.3 Hz, 1 H), 7.95 (dd, J = 2.3, 12.1 Hz, 1 H), 7.60 (m, 2 H), 7.39 (d, J = 8.6 Hz, 1 H), 7.22-7.27 (m, 3 H), 6.21 (dd, J = 1.0, 5.6 Hz, 1 H), 4.47 (t, J = 6.4 Hz, 2 H), 2.57 (t, J = 7.4 Hz, 2 H), 2.31 (s, 6 H), 2.09 (tt, J = 6.4, 7.4 Hz, 2 H) |
| 23 | 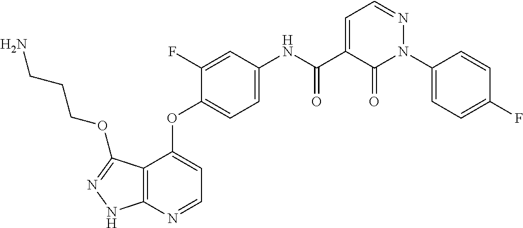 | N-(4-(3-(3- aminopropoxy)- 1H-pyrazolo[3,4-b] pyridin-4-yloxy)- 3-fluorophenyl)- 2-(4-fluorophenyl)- 3-oxo-2,3-dihydro- pyridazine- 4-carboxamide | ¹H NMR (400 MHz, CDCl₃) δ 11.82 (s, 1 H), 10.07 (br s, 1 H), 8.41 (d, J = 4.3 Hz, 1 H), 8.26 (d, J = 5.6 Hz, 1 H), 8.24 (d, J = 3.9 Hz, 1 H), 7.95 (d, J = 12.1 Hz, 1 H), 7.60 (m, 2 H), 7.38 (d, J = 9.4 Hz, 1 H), 7.21-7.26 (m, 3 H), 6.21 (d, J = 5.6 Hz, 1 H), 4.50 (m, 2 H), 2.92 (t, J = 6.2 Hz, 2 H), 2.02 (m, 2 H) |
| 24 | 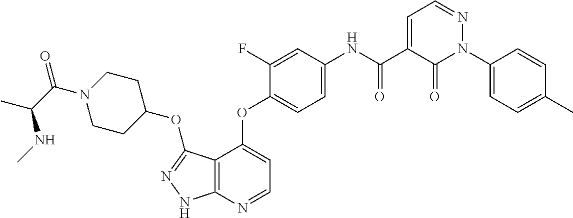 | (S)-N-(3-fluoro-4- (3-(1-(2-(methyl- amino)propanoyl) piperidin-4-yloxy)- 1H-pyrazolo[3,4- b]pyridin-4- yloxy)phenyl)-2- (4-fluorophenyl)- 3-oxo-2,3-dihydro- pyridazine- 4-carboxamide | ¹H NMR (400 MHz, CDCl₃) δ 11.82 (s, 1 H), 10.07 (br s, 1 H), 8.41 (d, J = 4.3 Hz, 1 H), 8.26 (d, J = 5.6 Hz, 1 H), 8.24 (d, J = 3.9 Hz, 1 H), 7.95 (d, J = 12.1 Hz, 1 H), 7.60 (m, 2 H), 7.38 (d, J = 9.4 Hz, 1 H), 7.21-7.26 (m, 3 H), 6.21 (d, J = 5.6 Hz, 1 H), 4.50 (m, 2 H), 2.92 (t, J = 6.2 Hz, 2 H), 2.02 (m, 2 H) |
| 25 | 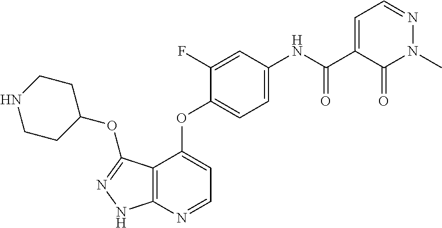 | N-(3-fluoro-4-(3- (piperidin-4-yloxy)- 1H-pyrazolo[3,4- b]pyridin-4- yloxy)phenyl)-2- methyl-3-oxo-2,3- dihydropyridazine- 4-carboxamide | ¹H NMR (400 MHz, CDCl₃) d 11.98 (s, 1 H), 8.31 (d, 1 H), 8.28 (d, 1 H), 8.08 (d, 1 H), 7.95 (dd, 1 H), 7.42 (d, 1 H), 7.24 (m, 1 H), 6.25 (d, 1 H), 4.97 (m, 1 H), 3.97 (s, 3 H), 3.16 (m, 2 H), 2.77 (m, 2 H), 2.14 (m, 2 H), 1.77 (m, 2 H) |

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A compound selected from Formulas Ia and Ib: 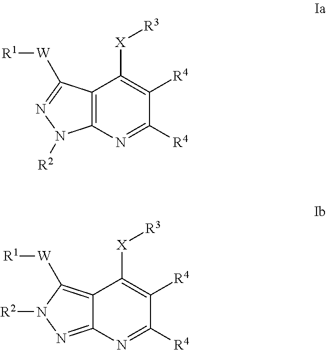
and pharmaceutically acceptable salts thereof, wherein:
X is O, S or NR¹⁰;
W is O, S, S(═O) or S(═O)₂;
R¹ is H, C₁-C₁₂ alkyl, C₂-C₈ alkenyl, C₂-C₈ alkynyl, —C(═O)NR¹⁰R¹¹, or —(CR¹⁴R¹⁵)ₜNR¹⁰R¹¹, or
R¹ is C₃-C₁₂ carbocyclyl, C₂-C₂₀ heterocyclyl, C₆-C₂₀ aryl, C₁-C₂₀ heteroaryl, (CR¹⁴R¹⁵)ₜC₃-C₁₂ carbocyclyl, (CR¹⁴R¹⁵)ₙC₂-C₂₀ heterocyclyl, (CR¹⁴R¹⁵)ₙC₆-C₂₀ aryl or (CR¹⁴R¹⁵)ₙC₁-C₂₀ heteroaryl, wherein said alkyl, alkenyl, alkynyl, carbocyclyl, heterocyclyl, aryl and heteroaryl are optionally substituted with one or more groups independently selected from R¹⁰, F, Cl Br, I, CN, CF₃, oxo, —OR¹⁰, SR¹⁰, —C(═Y)R¹⁰, —C(═Y)OR¹⁰, —C(═Y)NR¹⁰R¹¹, —(CR¹⁴R¹⁵)ₙ—NR¹⁰R¹¹, —NR¹⁰C(═Y)R¹³, —NR¹⁰C(═Y)OR¹¹, —NR¹²C(═Y)NR¹⁰R¹¹, —NR¹²SO¹⁰R¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰(OR¹¹), —OP(OR¹⁰)(OR¹¹), —S(O)R¹⁰, —S(O)₂R¹⁰, —S(O)₂NR¹⁰R¹¹, —S(O)(OR¹⁰, —S(O)₂(OR¹⁰), —SC(═Y)R¹⁰, —SC(═Y)OR¹⁰, —SC(═Y)NR¹⁰R¹¹, C₁-C₁₂ alkyl, (C₁-C₆ alkyl)OH, —(CH₂)ₙCH(OH)(CH₂)ₘOH, C₁-C₆ fluoroalkyl, C₂-C₈ alkenyl, C₂-C₈ alkynyl, C₃-C₁₂ cycloalkyl, C₂-C₂₀ heterocyclyl, —(CR¹⁴R¹⁵)ₙC₂-C₂₀ heterocyclyl, C₆-C₂₀ aryl, —(CR¹⁴R¹⁵)ₙC₆-C₂₀ aryl, C₁-C₂₀ heteroaryl, —(CR¹⁴R¹⁵)ₜNR¹⁰R¹¹, —NR¹⁰(CR¹⁰R¹¹)ₙCHR¹⁰R¹¹, —(CR¹⁴R¹⁵)—NR¹²C(═O)(CR¹⁴R¹⁵)NR¹⁰R¹¹, —(CR¹⁴R¹⁵)ₜNR¹⁰R¹¹, —C(═Y)(CR¹⁰R¹¹)ₙ—OR¹⁰, and —C(═Y)(CR¹⁰R¹¹)ₙ—NR¹⁰R¹¹, or
R¹ is NR^{x}R^{y};
R² is H, CF₃, CN, —C(═Y)R¹⁰, —C(═Y)OR¹⁰, —C(═Y)NR¹⁰R¹¹, —C(═O)NR¹²(CR¹⁴R¹⁵)ₜNR¹⁰R¹¹, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from F, Cl, Br, I, (CH₂)ₙOR¹⁰, (CH₂)ₙNR¹⁰R¹¹, heteroaryl and heterocyclyl;
R³ is C₃-C₁₂ carbocyclyl, C₂-C₂₀ heterocyclyl, C₆-C₂₀ aryl or C₁-C₂₀ heteroaryl, wherein said carbocyclyl, heterocyclyl, aryl and heteroaryl are optionally substituted with one or more groups independently selected from F, Cl, Br, I, CN, CF₃, OR¹⁰, SR¹⁰, —C(═Y)R¹⁰, —C(═Y)OR¹⁰, —C(═Y)NR¹⁰R¹¹, —NR¹⁰R¹¹, —NR¹⁰C(═Y)R¹³, —NR¹⁰C(═Y)OR¹¹, —NR¹²C(═Y)NR¹⁰R¹¹, —NR¹²C(═O)C(═O)R¹⁰R¹¹, —NR¹²C(═O)C(═O)OR^{a}, —NR¹²SO₂R¹⁰, —NR¹²C(═Y¹) (CR¹⁴R¹⁵)ₙC(═Y²)NR¹⁰R¹¹, —NR¹²C(═Y¹)NR¹⁰C(═Y²)(CR¹⁴R¹⁵)ₙR¹¹, —NR¹²C(═Y¹) (CR¹⁴R¹⁵)ₙC(═Y²)CR¹⁴R¹⁵)ₘR¹⁰, —OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl, wherein said alkyl, alkenyl, alkynyl, cycloalkyl, heterocyclyl, aryl and heteroaryl are optionally substituted with one or more groups independently selected from F, Cl, Br, I, OH, C₁-C₁₂ alkyl, NR¹⁰R¹¹, and (CR¹⁴R¹⁵)ₙ-aryl;
R⁴ is H, F, Cl, Br, CF₃, CN, —C(═Y)R¹⁰, —C(═Y)OR¹⁰, —C(═Y)NR¹⁰R¹¹, —NR¹⁰R¹¹, NR¹⁰C(═Y)R¹¹, NR¹⁰C(═Y)OR¹¹, NR¹²C(═Y)NR¹⁰R¹¹, —NR¹²SO₂NR¹⁰R¹¹, —OR¹⁰, —OC(═Y)R¹⁰, —OC(═Y)OR¹⁰, —OC(═Y)NR¹⁰R¹¹, —C(═O)NR¹²(CR¹⁴R¹⁵)ₜNR¹⁰R¹¹, —OP(═Y)(OR¹⁰)(OR¹¹), —OP(OR¹⁰)(OR¹¹), —SR¹⁰, —S(O)R¹⁰, —S(O)₂R¹⁰, —S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl;
R¹⁰, R¹¹ and R¹² are independently H, C₁-C₁₂ alkyl, C₂-C₈ alkenyl, C₂-C₈ alkynyl, OR^{a}, NR^{a}R^{b}, C₃-C₁₂ carbocyclyl, (CR¹⁴R¹⁵)ₙC₂-C₂₀ heterocyclyl, (CR¹⁴R¹⁵)ₙC₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more groups independently selected from F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₃-C₁₂ carbocyclyl, C₆-C₂₀ aryl, and C₁-C₂₀ heteroaryl,
or R¹⁰and R¹¹ together with the nitrogen to which they are attached optionally form a saturated, partially unsaturated or fully unsaturated C₃-C₂₀ heterocyclic ring optionally containing one or more additional ring atoms selected from N, O or S, wherein said heterocyclic ring is optionally substituted with one or more groups independently selected from oxo, (CH₂)ₙOR^{a}, NR^{a}R^{b}, CF₃, F, Cl, Br, I, SO₂R^{a}, C(═O)R^{a}, NR¹⁰C(═Y)R¹¹, C(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl,
or R¹⁰ and R¹² together with the atoms to which they are attached form an oxo-substituted C₃-C₂₀ heterocyclic ring optionally fused to a benzene ring;
R¹³ is H, C₁-C₁₂ alkyl, C₂-C₈ alkenyl, C₂-C₈ alkynyl, (CR¹⁴R¹⁵)ₙ-cycloalkyl, (CR¹⁴R¹⁵)ₙ-heterocyclyl, (CR¹⁴R¹⁵)ₙ-aryl, (CR¹⁴R¹⁵)ₙ-heteroaryl, (CR¹⁴R¹⁵)ₙ—O—(CR¹⁴R¹⁵)ₘ-aryl, (CR¹⁴R¹⁵)ₙ—OR¹⁰, (CR¹⁴R¹⁵)ₙ—NR¹⁰R¹¹, (CR¹⁴R¹⁵)ₙ—NR¹⁰C(═O)R¹¹, or (CR¹⁴R¹⁵)ₙ—NR¹⁰(SO₂Me)—R¹¹, wherein said alkyl, alkenyl, alkynyl, cycloalkyl, heterocycloalkyl, and heteroaryl portions are optionally substituted with one or more groups independently selected from F, Cl, Br, I, oxo, SO₂R^{c}, CN, OR^{a}, C(═O)R^{a}, C(═O)OR^{a}, NR^{a}R^{b}, NR^{a}C(═O)R^{b}, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl, and C₁-C₂₀ heteroaryl, wherein said alkyl, C₂-C₈ alkenyl, C₂-C₈ alkynyl, C₃-C₁₂ cycloalkyl, C₂-C₂₀ heterocyclyl, C₆-C₂₀ aryl, and C₁-C₂₀ heteroaryl are optionally substituted with one or more groups independently selected from F, Cl, Br, and I;
each R¹⁴ and R¹⁵ is independently H, C₁-C₁₂ alkyl, or (CH₂)ₜ-aryl,
or R¹⁴ and R¹⁵ together with the atoms to which they are attached form a saturated or partially unsaturated C₃-C₁₂ carbocyclic ring,
or R¹⁰ and R¹⁵ together with the atoms to which they are attached form an oxo-substituted saturated or partially unsaturated monocyclic or bicyclic C₁-C₂₀ heterocyclic ring optionally further substituted with one or more groups independently selected from F, Cl, Br, I, OR^{a}, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl, or C₁-C₂₀ heteroaryl, wherein said alkyl and aryl are optionally substituted with one or more groups independently selected from F, Cl, Br, and I,
or R¹⁴ is null and R¹⁰ and R¹⁵ together with the atoms to which they are attached form a C₁-C₂₀ heteroaryl ring having one or more heteroatoms;
R^{a} and R^{b} are independently H, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl, wherein said alkyl, alkenyl, alkynyl, carbocyclyl, heterocyclyl, aryl, and heteroaryl are optionally substituted with one or more alkyl groups;
R^{c} is C₁-C₁₂ alkyl or C₆-C₂₀ aryl, wherein said alkyl and aryl are optionally substituted with one or more groups independently selected from F, Cl, Br, I, OR^{a} and C(═O)NR^{a}R^{b};
R^{x} is H or C₁-C₆ alkyl;
R^{y} is (i) (C₁-C₆ alkyl)NR^{j}R^{k} wherein R^{j} and R^{k} are independently H or C₁-C₆ alkyl; (ii) C₅-C₆ cycloalkyl optionally substituted with OH or —OC(═O)CF₃; or (iii) a 5-6 membered heterocyclic ring having 1 to 2 ring heteroatoms independently selected from N and O and optionally substituted with a halogen group, C₁-C₆ alkyl, (C₁-C₆ alkyl)OH, (C₁-C₆ alkyl)O(C₁-C₆ alkyl), or C₁-C₆ fluoroalkyl;
Y, Y¹ and Y² are independently O or S;
t is 1, 2, 3, 4, 5 or 6; and
n and m are independently 0, 1, 2, 3, 4, 5 or 6.

2. The compound of claim 1, wherein R² is C₁-C₆ alkyl or H.

3. The compound of claim 1, wherein X is O or NR¹⁰.

4. (canceled)

5. The compound of claim 1, wherein X is the structure:

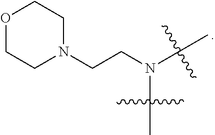

6. The compound of claim 3, wherein R¹ is
(i) phenyl optionally substituted with halogen, C₁-C₆ alkyl, C(═O)C₁-C₆ alkyl, C(═O)(C₃-C₆ cycloalkyl), C(═O)O(C₁-C₆ alkyl), CH₂-heteroaryl (wherein said heteroaryl is a 5 membered ring having 2-3 ring nitrogen atoms), CH₂-hetCyc (wherein hetCyc is a 6 membered ring having 1 to 2 ring heteroatoms independently selected from N and O and optionally substituted with C₁-C₆ alkyl), C(═O)NH(CH₂)₂-hetCyc wherein hetCyc is a 6 membered ring having 1 to 2 ring heteroatoms independently selected from N and O), SO₂NH(C₁-C₆ alkyl), NMeOMe, C(═O)NR^{h}Rⁱ, or NR^{h}Rⁱ wherein R^{h} and Rⁱ are independently H or C₁-C₆ alkyl, or
(ii) a 5-6 membered heteroaryl having 1 or 2 ring heteroatoms selected from N and O and optionally substituted with one or more groups independently selected from C(═O)NH(C₁-C₆ alkyl) R¹⁰, Br, hetCyc and CH₂-hetCyc, wherein hetCyc is a 6 membered heterocyclic ring having a ring nitrogen atom and optionally having a second ring heteroatom selected from N and O, wherein hetCyc is optionally substituted with C₁-C₆ alkyl or (C₁-C₆ alkyl)OH.

7. The compound of claim 6, wherein R¹ is selected from:

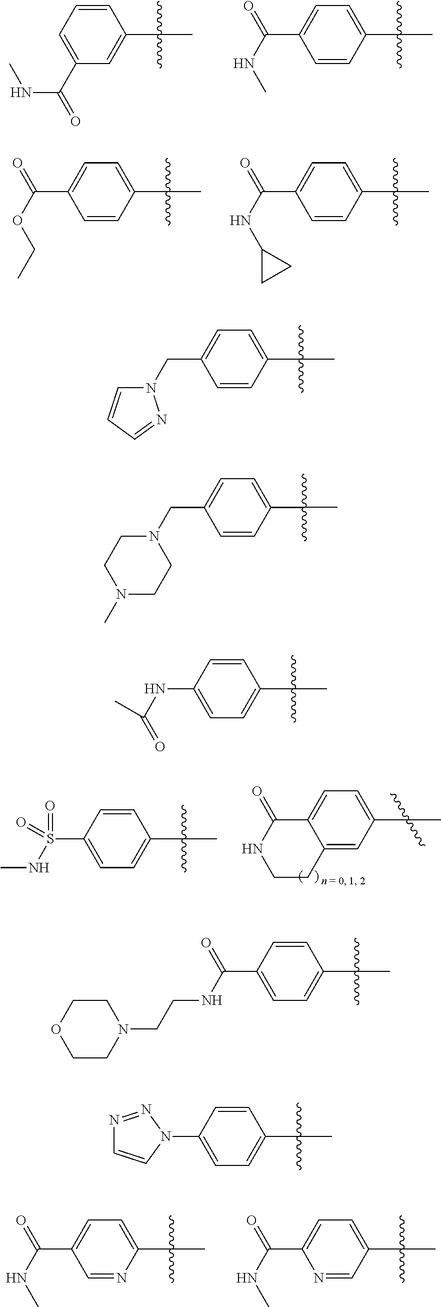

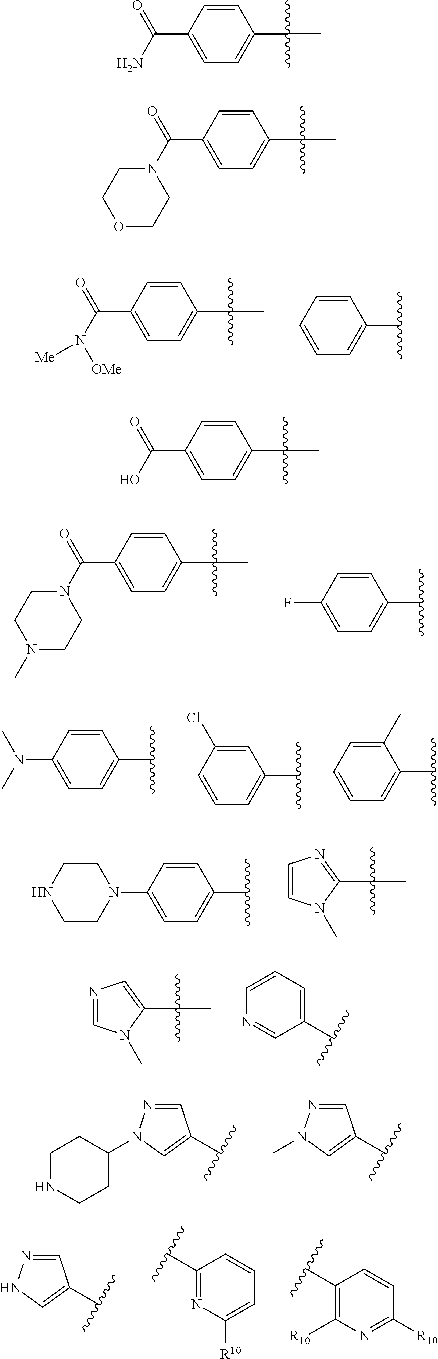

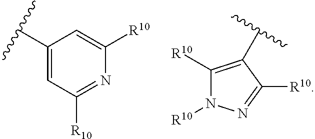

8.-9. (canceled)

10. The compound of claim 3, wherein R¹ is —C(═O)NR¹⁰R¹¹, —(CR¹⁴R¹⁵)ₜNR¹⁰R¹¹, C₁-C₁₂ alkyl or C₃-C₁₂ carbocyclyl, wherein said alkyl and carbocyclyl are optionally substituted with one or more groups independently selected from OR¹⁰, NR¹⁰R¹¹, NR¹⁰(CR¹⁰R¹¹)ₙCHR¹⁰R¹¹, heterocycl and heteroaryl.

11. The compound of claim 10, wherein R¹ is selected from methyl, CH₂OH, CH₂CH₂OH, CH₂CH₂CH₂OH, CH(OH)CH₂OH, CH,CH(OH)CH₂OH, CH₂CH₂CH₂N(CH₃)₂, —CH₂CH₂CH₂NH₂,

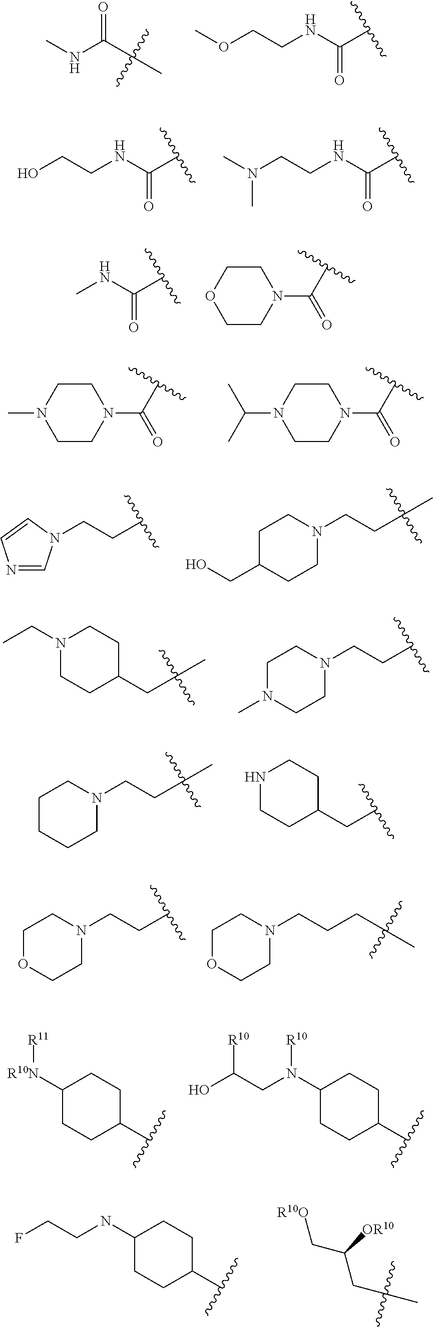

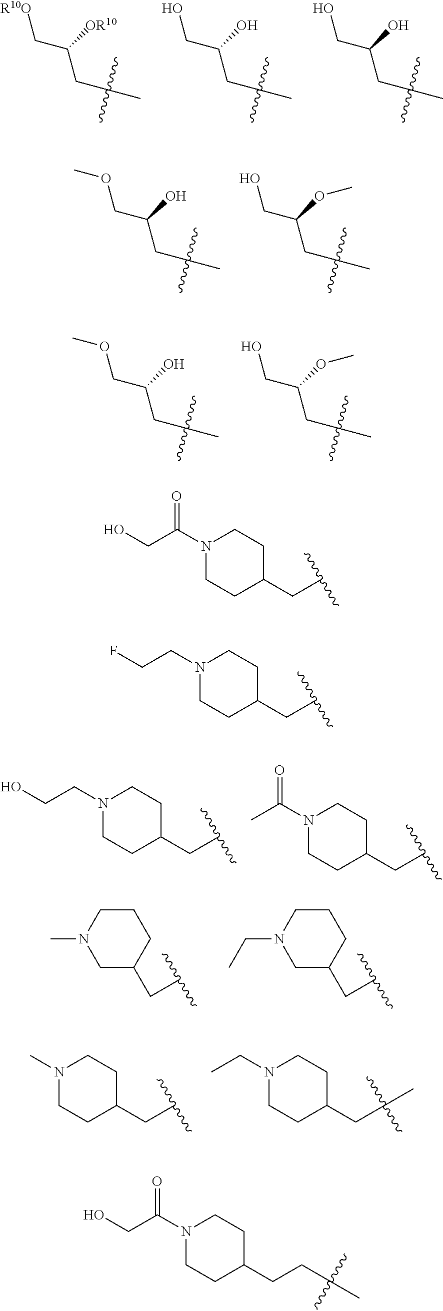

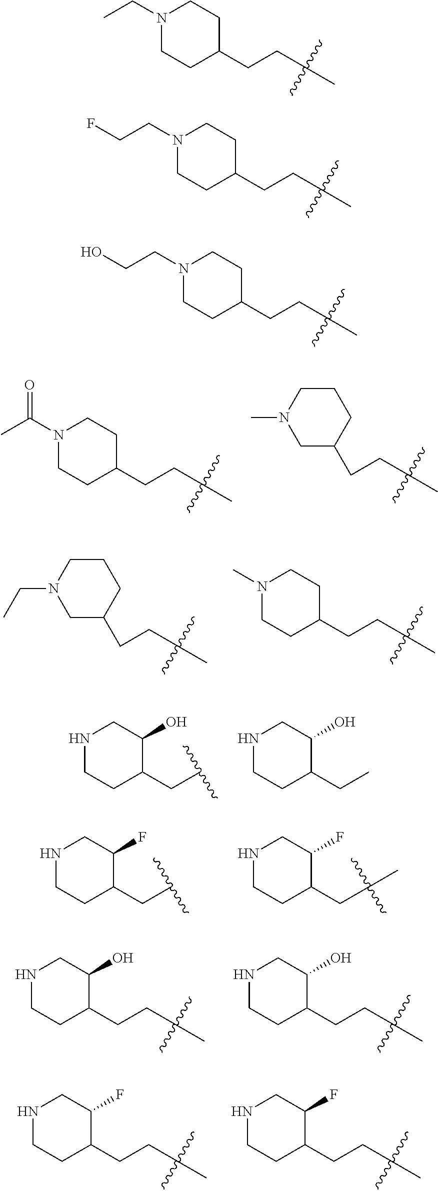

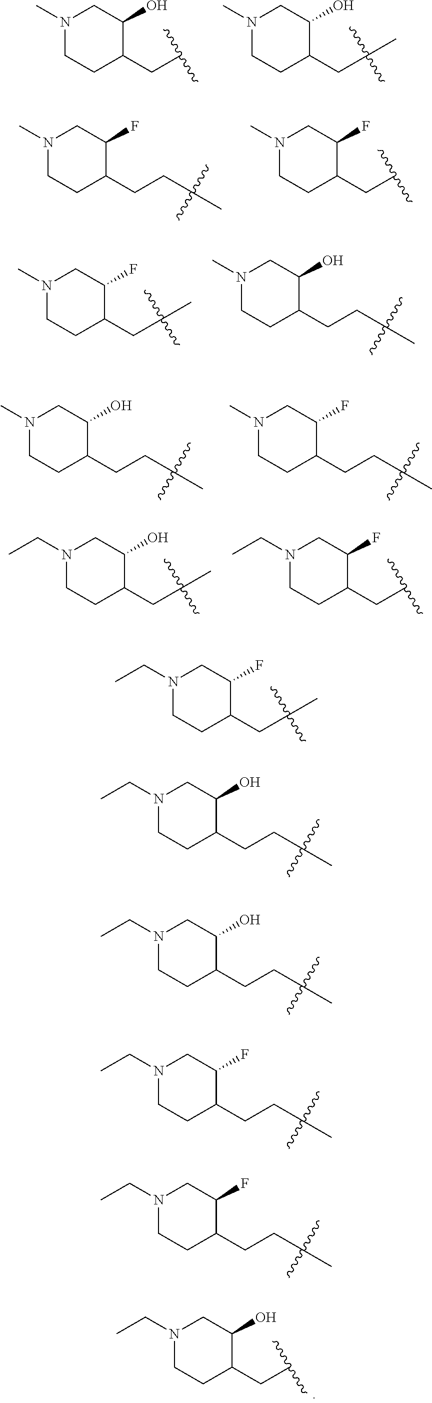

12.-15. (canceled)

16. The compound of claim 3, wherein R¹ is a saturated or partially unsaturated 5-10 membered monocyclic or bicyclic heterocyclic ring, wherein said ring has one or two ring atoms independently selected from N and O and is optionally substituted with R¹⁰, C₁-C₆ alkyl, (C₁-C₆ alkyl)O(C₁-C₆ alkyl), halo, OR¹⁰, —C(═O)(CR¹⁰R¹¹)ₙ—OR¹⁰, —C(═O)(CR¹⁰R¹¹)ₙ—NR¹⁰R¹¹, (C₁-C₆ alkyl)OH, C₁-C₆ fluoroalkyl, NR¹⁰R¹¹ or CH₂NR¹⁰R¹¹ wherein R¹⁰ and R¹¹ are independently H, C₁-C₆ alkyl, (C₁-C₆ alkyl)OH, hetCyc or CH₂hetCyc, wherein hetCyc is a 5-6 membered ring having one or two ring nitrogen atoms.

17. The compound of claim 16, wherein R¹ is:

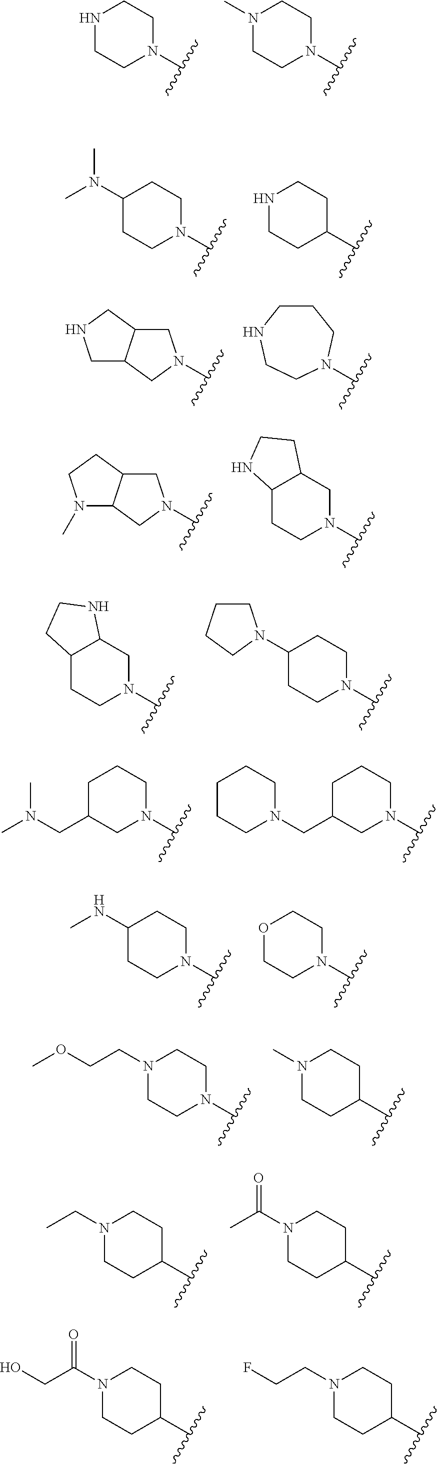

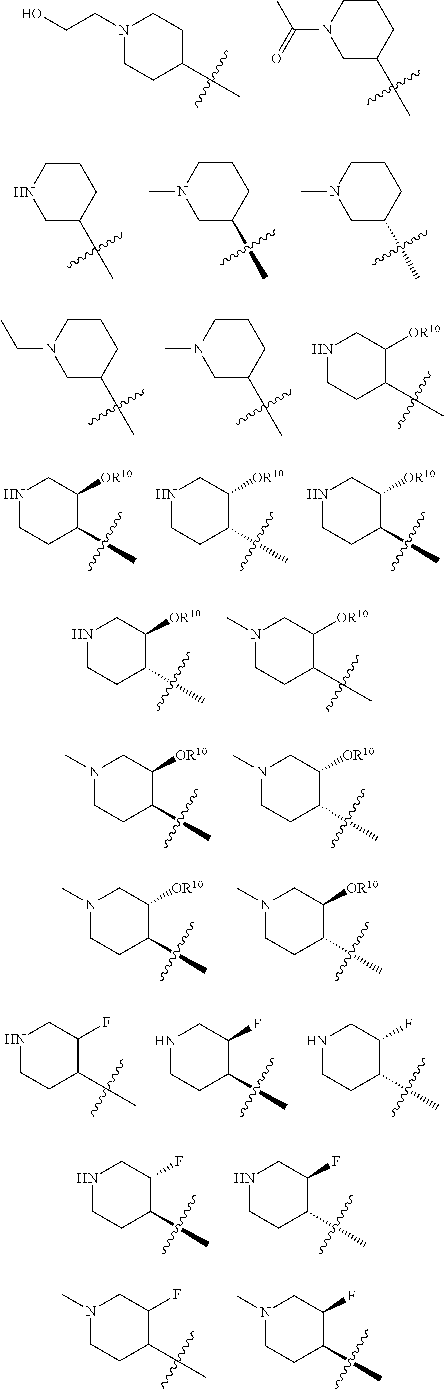

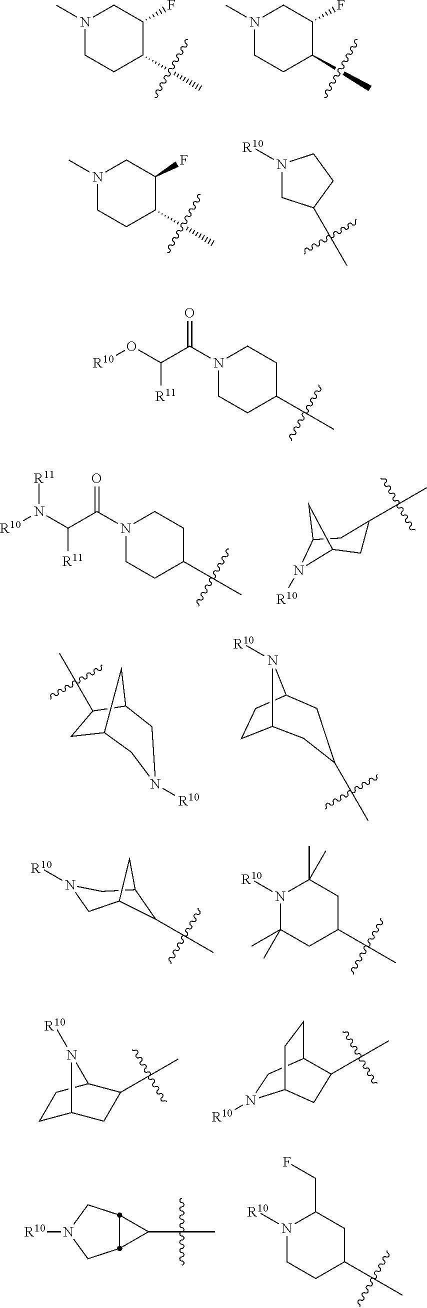

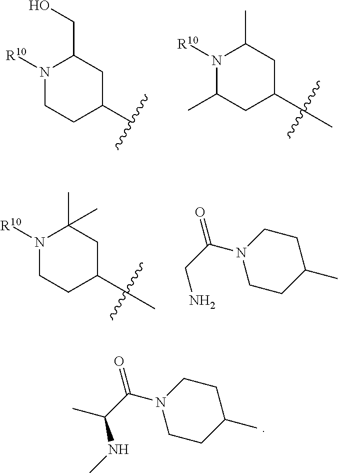

18. The compound of claim 3, wherein R¹ is NR^{x}R^{y}.

19. The compound of claim 18, wherein R¹ is

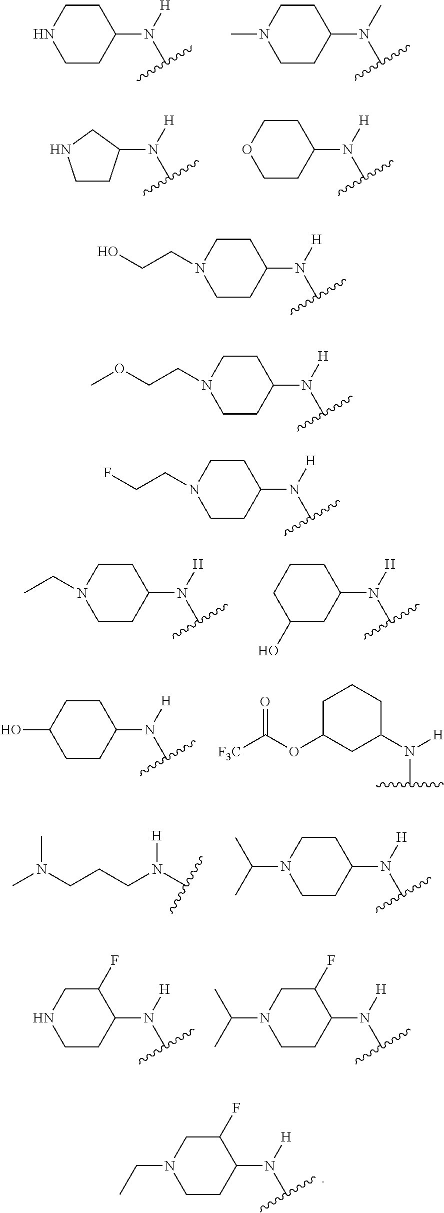

20.-21. (canceled)

22. The compound of claim 3, wherein:
R³ is 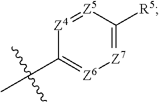
Z⁴, Z⁵, Z⁶, and Z⁷ are independently CR^{4a} or N and 0, 1, or 2 of Z⁴, Z⁵, Z⁶, and Z⁷ is N, wherein when Z⁴ and Z⁵ or Z⁶ and Z⁷ are CR^{4a}, then Z⁴ and Z⁵ or Z⁶ and Z⁷ optionally form a saturated, partially unsaturated or fully unsaturated carbocyclic or heterocyclic ring;
each R^{4a} is independently H, F, Cl, Br, CF₃, CN, —C(═Y)R¹⁰, —C(═Y)OR¹⁰, —C(═Y)NR¹⁰R¹¹, —NR¹⁰R¹¹, NR¹⁰C(═Y)OR¹¹, NR¹⁰C(═Y)NR¹⁰R¹¹, —NR¹²C(═Y)OR¹⁰, —NR¹²SO₂NR¹⁰R¹¹, —OR¹⁰, —OC(═Y)R¹⁰, —OC(═Y)OR¹⁰, —OC(═Y)NR¹⁰R¹¹, —C(═O)NR¹²(CR¹⁴R¹⁵)ₜNR¹⁰R¹¹, —OP(═Y)(OR¹⁰)(OR¹¹), —OP(OR¹⁰)(OR¹¹), —SR¹⁰, —S(O)R¹⁰, —S(O)₂R¹⁰, —S(O)₂NR¹⁰R¹¹, —SC(═Y)R¹⁰, —SC(═Y)OR¹⁰, C₁-C₁₂ alkyl, C₂-C₈ alkenyl, C₂-C₈ alkynyl, C₃-C₁₂ carbocyclyl, C₂-C₂₀ heterocyclyl, C₆-C₂₀ aryl, or C₁-C₂₀ heteroaryl; and
R⁵ is F, Cl Br, I, CN, CF₃, OR¹⁰, SR¹⁰, —C(═Y)R¹⁰, —C(═Y)OR¹⁰, —C(═Y)NR¹⁰R¹¹, —NR¹⁰R¹¹, —NR¹⁰C(═Y)R¹³, —NR¹⁰C(═Y)OR¹¹, —NR¹²C(═Y)NR¹⁰R¹¹, —NR¹²C(═O)C(═O)R¹⁰R¹¹, —NR¹²C(═O)C(═O)OR^{a}, —NR¹²SO₂R¹⁰, —NR¹²C(═Y¹)(CR¹⁴R¹⁵)ₙC(═Y²)NR¹⁰R¹¹, —NR¹²C(═Y¹)NR¹⁰C(═Y²)(CR¹⁴R¹⁵)ₙR¹¹, —NR¹²C(═Y¹)(CR¹⁴R¹⁵)ₙC(═Y²)(CR¹⁴R¹⁵)ₘR¹⁰, ——OC(═Y)R¹⁰, —OC(═Y)OR¹⁰, —OC(═Y)NR¹⁰R¹¹, —OS(O)₂(OR¹⁰), —OP(═Y)(OR¹⁰)(OR¹¹), —OP(OR¹⁰)(OR¹¹), —S(O)R¹⁰, —S(O)₂R¹⁰, —S(O)₂NR¹⁰R¹¹, —S(O)(OR¹⁰), —S(O)₂(OR¹⁰), —SC(═Y)R¹⁰, —SC(═Y)OR¹⁰, —SC(═Y)NR¹⁰R¹¹, C₁-C₁₂ alkyl, C₂-C₈ alkenyl, C₂-C₈ alkynyl, C₃-C₁₂ cycloalkyl, C₂-C₂₀ heterocyclyl, C₆-C₂₀ aryl and C₁-C₂₀ heteroaryl, wherein said alkyl, alkenyl, alkynyl, cycloalkyl, heterocyclyl, aryl and heteroaryl are optionally substituted with one or more groups independently selected from alkyl, NR¹⁰R¹¹, and (CR¹⁴R¹⁵)ₙ-aryl.

23. The compound of claim 22, wherein R³ is selected from the structures: 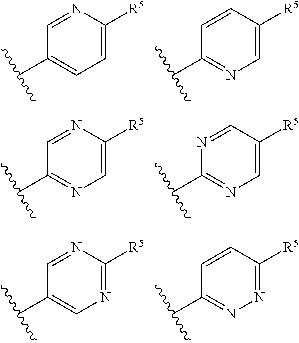
and substituted forms thereof.

24. The compound of claim 23, wherein R³ is selected from the structures

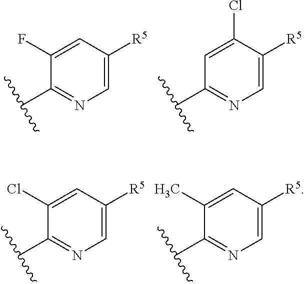

25. The compound of claim 22, wherein
R³ is 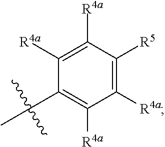
and
each R^{4a} is independently H, F, Cl, C₁-C₆ alkyl, O—(C₁-C₆ alkyl), or CN.

26. The compound of claim 25, wherein R³ is selected from the structures:

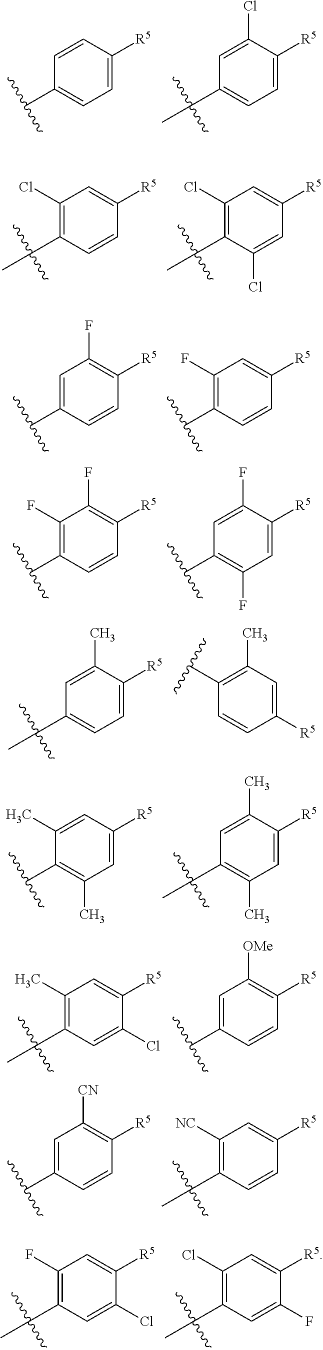

27. (canceled)

28. The compound of claim 22, wherein R⁵ is

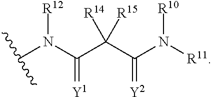

29. The compound of claim 28, wherein R¹¹ is an aryl group optionally substituted with F.

30. The compound of claim 28, wherein R¹⁴ and R¹⁵ together with the atom to which they are attached form an optionally substituted carbocyclic ring.

31. The compound of claim 28, wherein R¹⁵ and R¹⁰together with the atoms to which they are attached form an oxo-substituted 5, 6 or 7 membered monocyclic or bicyclic heterocycle.

32. The compound of claim 22, wherein R⁵ is

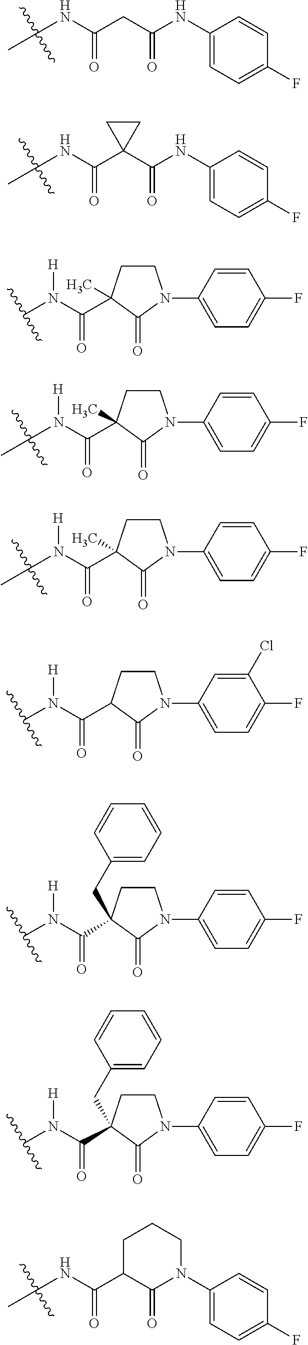

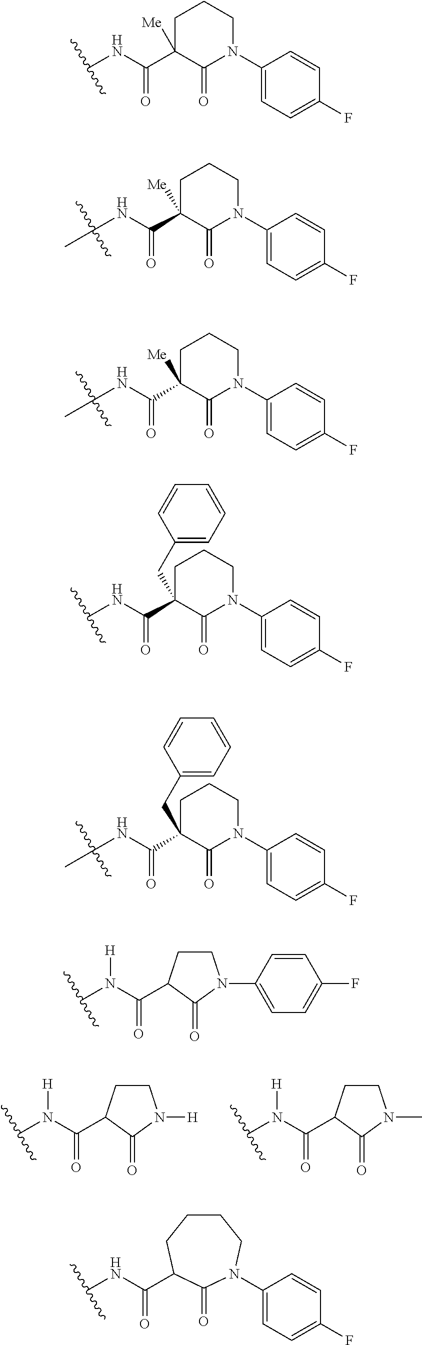

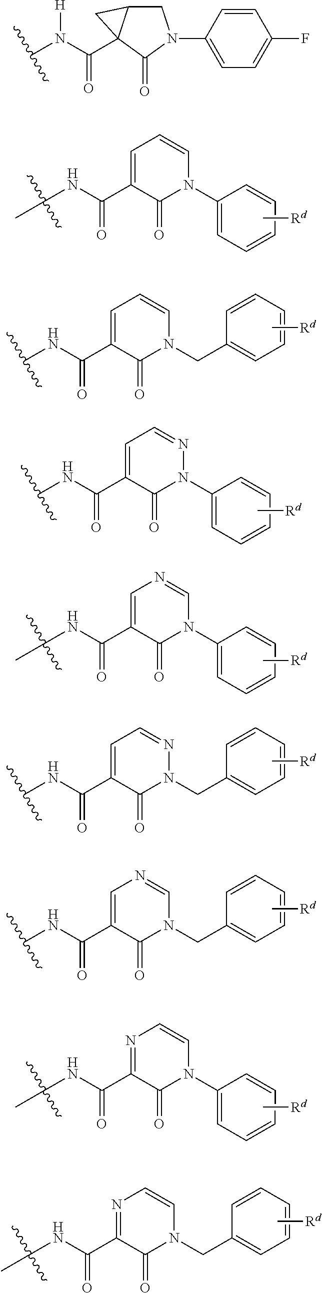

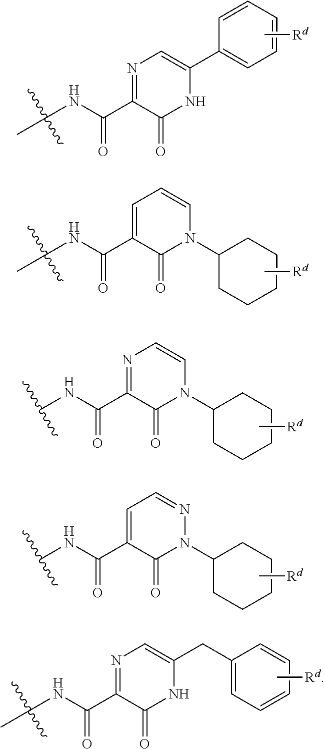

33.-34. (canceled)

35. The compound of claim 22, wherein R⁵ has the structure: 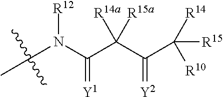
wherein R^{14a} and R^{15a} are H, or R¹⁴ and R¹⁵ together with the atoms to which they are attached form a cyclopropylidine group.

36. The compound of claim 35, wherein R¹⁰ is phenyl or CH₂-phenyl optionally substituted with a halogen group.

37. The compound of claim 22, wherein R⁵ is selected from the structures:

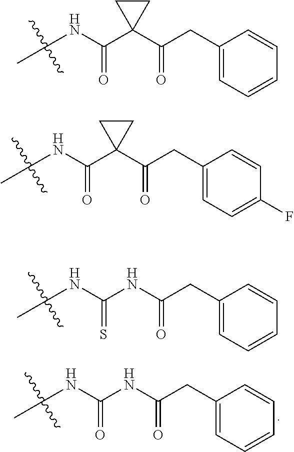

38.-39. (canceled)

40. The compound of claim 22, wherein R⁵ has the structure: 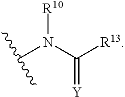
wherein R¹³ is:
(i) C₁-C₆ alkyl;
(ii) (CR¹⁴R¹⁵)—O—(CH₂)ₘ-phenyl, wherein said phenyl is optionally substituted with halogen, R¹⁴ and R¹⁵ are independently H or methyl, and m is 0 or 1;
(iii) OR^{a}, wherein R¹ is C₁-C₆ alkyl or phenyl;
(iv) (C₁-C₃ alkyl)-phenyl;
(v) (C₁-C₂ alkyl)-hetAr wherein hetAr is a 6 membered heteroaryl ring having one or two ring nitrogen atoms. A particular example of R¹³ is (C₁-C₂ alkyl)-pyridyl;
(vi) a 5-6 membered heteroaryl ring having 1 to 2 ring atoms independently selected from N, O and S and optionally substituted with one or two groups independently selected from NH-phenyl, morpholinyl, phenyl, and C₁-C₆ alkyl;
(vii) phenyl optionally substituted with one or two groups independently selected from CN, F, O-phenyl, N(C₁-C₆ alkyl)₂, and NHC(═O)(C₁-C₆ alkyl);
(viii) CH₂—N(C₁-C₄ alkyl)SO₂R^{a} or CH₂—N(CH₂Ph)SO₂R^{a}, wherein R^{a} is C₁-C₆ alkyl, phenyl or a 5 membered heteroaryl ring having one or two ring heteroatoms independently selected from N and O and optionally substituted with C₁-C₆ alkyl;
(ix) (CH₂)ₙ-hetCyc wherein n is 0 or 1 and hetcyc is a saturated or partially saturated 6 membered heterocyclic ring having a ring nitrogen atom and optionally substituted with oxo, C(═O)(C₁-C₆ alkyl), SO₂—(C₁-C₆ alkyl), SO₂-phenyl or C(═O)O(C₁-C₆ alkyl);
(x) C₁-C₆ alkyl optionally substituted with (C₃-C₆)cycloalkyl or O—(C₁-C₆ alkyl);
(xi) CH₂N(C₁-C₆ alkyl)C(═O)phenyl; or
(xii) (CR¹⁴R¹⁵)hetAr.

41. The compound of claim 40, wherein R⁵ is selected from the structures:

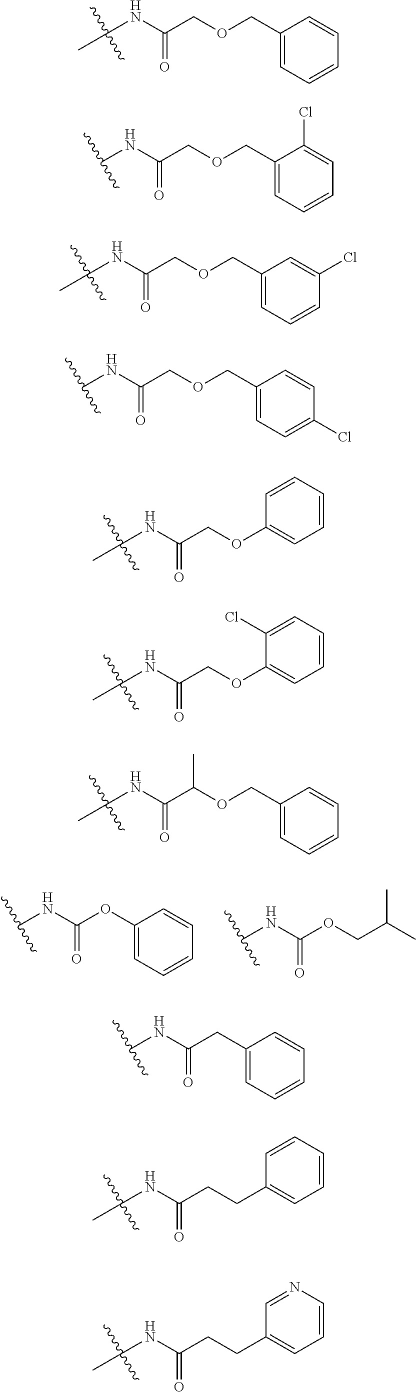

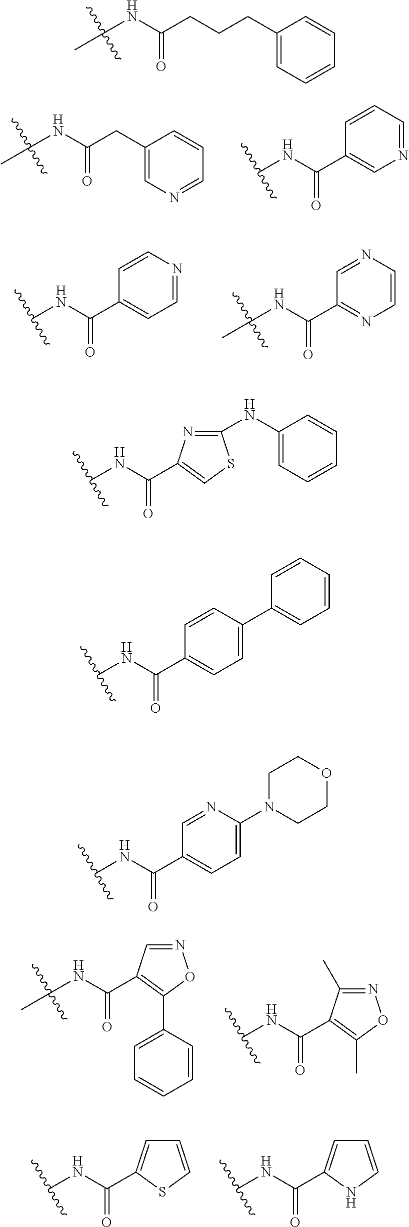

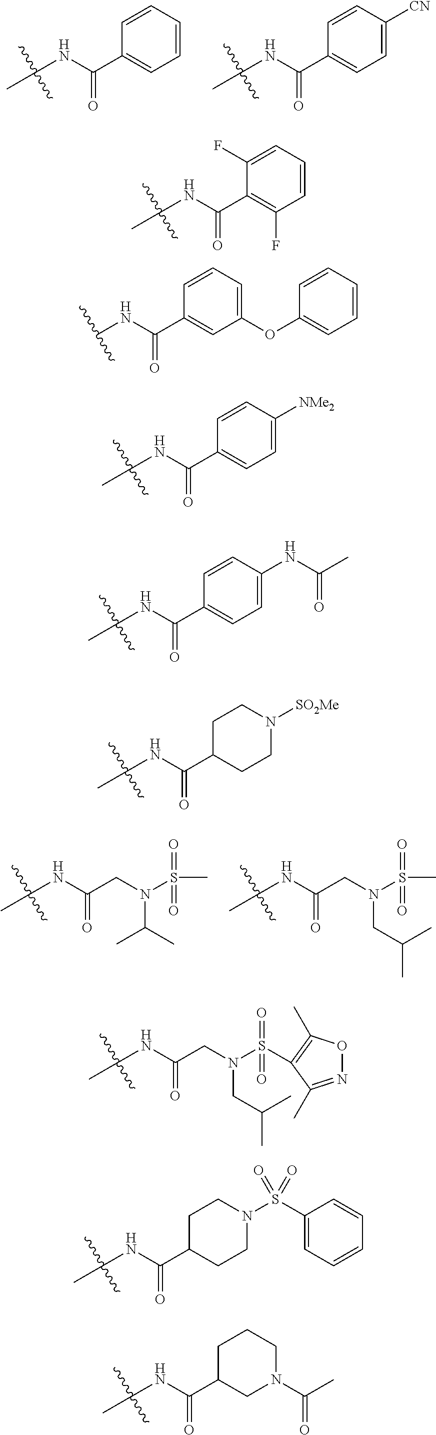

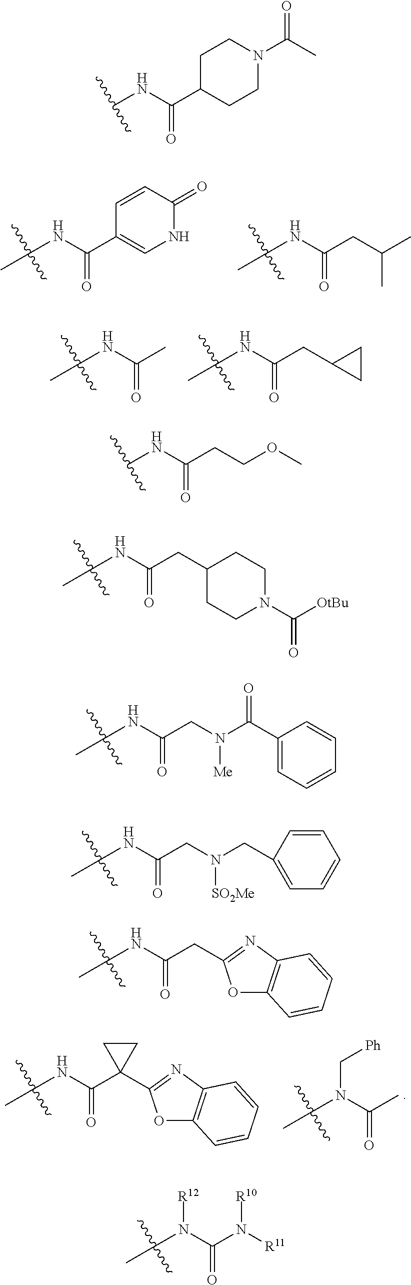

42. The compound of claim 22, wherein R⁵ is
wherein R¹¹ is aryl or heteroaryl optionally substituted with C₁-C₆ alkyl.

43. The compound of claim 42, wherein R⁵ is selected from the structures:

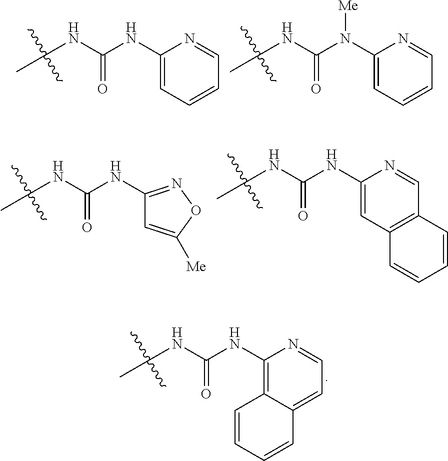

44. The compound of claim 42, wherein R¹⁰and R¹² together with the atoms to which they are attached form an oxo-substituted 6 membered heterocyclic ring, wherein said heterocyclic ring is optionally fused to a phenyl ring.

45. The compound of claim 44, wherein R⁵ is selected from the structures:

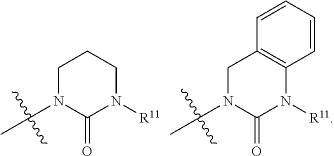

46. The compound of claim 22, wherein R⁵ is NR¹²SO₂R¹⁰, NR¹²C(═O)C(═O)NR¹⁰R¹¹ or NR¹²C(═O)C(═O)OR^{a}, wherein R¹⁰ is phenyl optionally substituted with halogen, O—(C₁-C₆ alkyl), or C(═O)NH(C₁-C₆ alkyl).

47. The compound of claim 46, wherein R⁵ is selected from the structures:

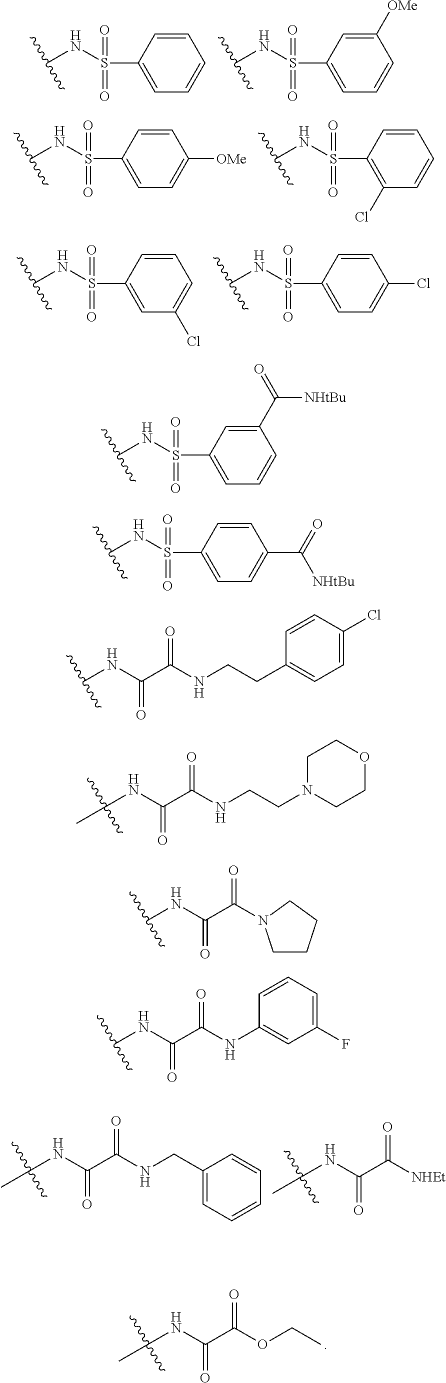

48.-52. (canceled)

53. The compound of claim 52 claim 22, wherein R⁵ is selected from the structures: 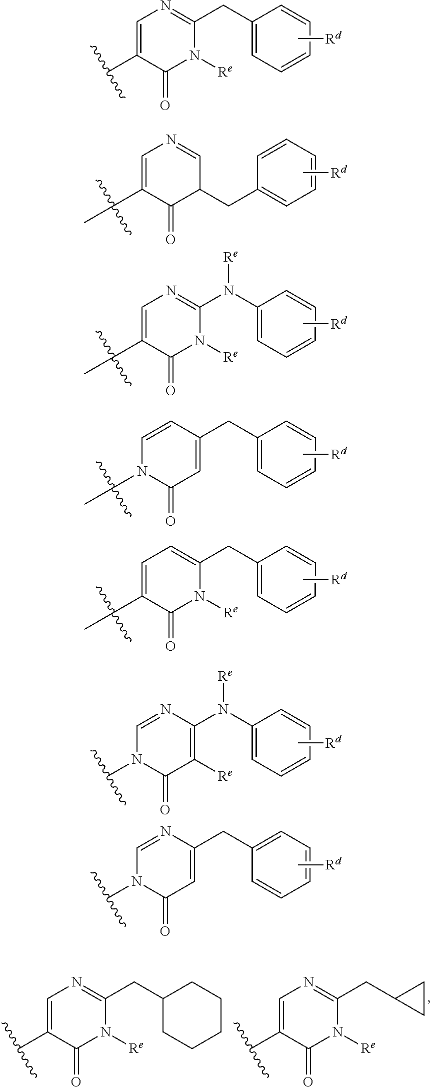
wherein:
R^{d} is F, Cl, Br, I, SO₂R^{c}, CN, OR^{a}, NR^{a}R^{b}, C(═O)NR^{a}R^{b}, CR^{a}C(═O)R^{b}, C₁-C₁₂ alkyl, C₂-C₈ alkenyl, C₂-C₈ alkynyl, C₆-C₂₀ aryl, and C₁-C₂₀ heteroaryl; and
each R^{e} is independently H or C₁-C₄ alkyl.

54. The compound of claim 22, wherein R⁵ is NR¹⁰R^{11,} wherein R¹⁰ is H and R¹¹ is hetAr, wherein hetAr is a substituted or unsubstituted 5-6 membered heteroaryl group having at least one ring nitrogen atom and optionally having a second ring heteroatom selected from N and O, and hetAr is optionally substituted with one or two groups independently selected from C₁-C₆ alkyl and C(═O)NR^{a}R^{b}.

55. The compound of claim 54, wherein R⁵ is

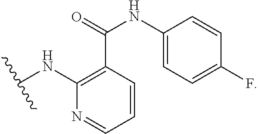

56. A compound of Formula 1 as defined in claim 1 and named in Examples 1-18.

57. A pharmaceutical composition comprising a compound according to claim 1 and a pharmaceutically acceptable carrier.

58. (canceled)

59. A method of treating a disease or condition selected from the group consisting of cancer in a patient, comprising administering to said patient a compound of claims 1.

60.-65. (canceled)

66. The compound of claim 1, wherein W is O.

67.-70. (canceled)
